SEQUENCE LISTING (16 sequences)

SEQ ID NO: 1 - RNA, 21 nt, synthetic construct
  modified_base 20..21: mod_base = OTHER; note = thymine
  source 1..21: mol_type = other RNA
ccaaaagagagactggatttt

SEQ ID NO: 2 - RNA, 21 nt, synthetic construct
  modified_base 20..21: mod_base = OTHER; note = thymine
  source 1..21: mol_type = other RNA
aatccagtctctcttttggtt

SEQ ID NO: 3 - RNA, 21 nt, synthetic construct
  modified_base 20..21: mod_base = OTHER; note = thymine
  source 1..21: mol_type = other RNA
ccaaaagagagactggatttt

SEQ ID NO: 4 - RNA, 21 nt, synthetic construct
  modified_base 20..21: mod_base = OTHER; note = thymine
  source 1..21: mol_type = other RNA
aatccagtctctcttttggtt

SEQ ID NO: 5 - RNA, 21 nt, synthetic construct
  modified_base 20..21: mod_base = OTHER; note = thymine
  source 1..21: mol_type = other RNA
ggatcatctcaagtcttactt

SEQ ID NO: 6 - RNA, 21 nt, synthetic construct
  modified_base 20..21: mod_base = OTHER; note = thymine
  source 1..21: mol_type = other RNA
gtaagacttgagatgatcctt

SEQ ID NO: 7 - RNA, 21 nt, synthetic construct
  modified_base 20..21: mod_base = OTHER; note = thymine
  source 1..21: mol_type = other RNA
ggtctcaagccactcgttttt

SEQ ID NO: 8 - RNA, 21 nt, synthetic construct
  modified_base 20..21: mod_base = OTHER; note = thymine
  source 1..21: mol_type = other RNA
aaacgagtggcttgagacctt

SEQ ID NO: 9 - RNA, 21 nt, synthetic construct
  modified_base 20..21: mod_base = OTHER; note = thymine
  source 1..21: mol_type = other RNA
ctaacaaccacacagaatatt

SEQ ID NO: 10 - RNA, 21 nt, synthetic construct
  modified_base 20..21: mod_base = OTHER; note = thymine
  source 1..21: mol_type = other RNA
tattctgtgtggttgttagtt

SEQ ID NO: 11 - RNA, 21 nt, synthetic construct
  modified_base 20..21: mod_base = OTHER; note = thymine
  source 1..21: mol_type = other RNA
gtctagacatgttcagctttt

SEQ ID NO: 12 - RNA, 21 nt, synthetic construct
  modified_base 20..21: mod_base = OTHER; note = thymine
  source 1..21: mol_type = other RNA
aagctgaacatgtctagactt

SEQ ID NO: 13 - RNA, 21 nt, synthetic construct
  modified_base 20..21: mod_base = OTHER; note = thymine
  source 1..21: mol_type = other RNA
atggcaaagaacaaataattt

SEQ ID NO: 14 - RNA, 21 nt, synthetic construct
  modified_base 20..21: mod_base = OTHER; note = thymine
  source 1..21: mol_type = other RNA
attatttgttctttgccattt

SEQ ID NO: 15 - RNA, 21 nt, synthetic construct
  modified_base 20..21: mod_base = OTHER; note = thymine
  source 1..21: mol_type = other RNA
ccgaagaaatgtttgtgaatt

SEQ ID NO: 16 - RNA, 21 nt, synthetic construct
  source 1..21: mol_type = other RNA
  modified_base 20..21: mod_base = OTHER; note = thymine
ttcacaaacatttcttcggtt

(19) United States
(12) Patent Application Publication
(10) Pub. No.: US 2025/0319037 A1
(43) Pub. Date: Oct. 16, 2025

(54) METHODS OF DELIVERING THERAPEUTIC AGENTS, AND LIPID COMPOSITIONS

(71) Applicants: FUJIFILM Corporation, Tokyo (JP); Massachusetts Institute of Technology, Cambridge, MA (US)

(72) Inventors: Sho TOYONAGA, Cambridge, MA (US); Daniel Griffith ANDERSON, Framingham, MA (US); Theresa Marie RAIMONDO, Greenville, RI (US); Dennis Zheng SHI, Somerville, MA (US)

(73) Assignees: FUJIFILM Corporation, Tokyo (JP); Massachusetts Institute of Technology, Cambridge, MA (US)

(21) Appl. No.: 19/240,405

(22) Filed: Jun. 17, 2025

Related U.S. Application Data
(63) Continuation of application No. PCT/JP2023/045247, filed on Dec. 18, 2023.
(60) Provisional application No. 63/433,629, filed on Dec. 19, 2022.

(51) Int. Cl. A61K 9/51; A61K 31/713; C12N 15/113

(52) U.S. Cl. CPC A61K 9/5123; A61K 31/713; C12N 15/113; C12N 2310/14

(57) ABSTRACT

The object of the present invention is to provide a method for delivering a therapeutic agent to endothelial cells, mesenchymal cells, or cancer cells which can realize excellent delivery efficiency to organs other than the liver, and a composition containing a therapeutic agent and lipid nanoparticles which can realize excellent delivery efficiency to an organ other than the liver. The present invention provides a method for delivering a therapeutic agent to endothelial cells, mesenchymal cells, or cancer cells, which comprises administering a lipid composition to a subject, wherein the lipid composition comprises the therapeutic agent and lipid nanoparticle,
and wherein the lipid nanoparticle comprises an ionizable lipid and a compound represented by formula (1) or a salt thereof.

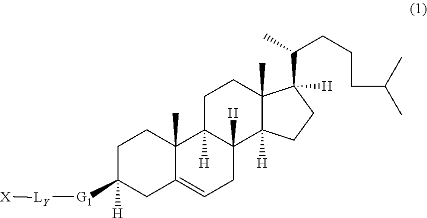

wherein G₁ represents —C(O)—, —OC(O)—, —O(CO)O— or —C(O)O—,
L_{Y} represents a single bond, an alkylene group having 1-14 carbon atoms, a substituted alkylene group having 1-14 carbon atoms, a heteroalkylene group having 1-14 carbon atoms, and a substituted heteroalkylene group having 1-14 carbon atoms.
X represents a basic functional group.

### CROSS-REFERENCE TO RELATED APPLICATIONS

This application is a Continuation of PCT International Application No. PCT/JP2023/045247 filed on Dec. 18, 2023, which claims priority under 35 U.S.C. § 119(a) to U.S. Provisional Patent Application No. 63/433,629 filed on Dec. 19, 2022. Each of the above application(s) is hereby expressly incorporated by reference, in its entirety, into the present application.

### INCORPORATION BY REFERENCE OF SEQUENCE LISTING

The instant application contains a Sequence Listing which has been filed electronically in xml format and is hereby incorporated by reference in its entirety. Said xml file, created on Jun. 13, 2025, is named Q309485_sequence_listing_as_filed.xml and is 23,152 bytes in size.

### TECHNICAL FIELD

The present invention relates to a method for delivering a therapeutic agent to endothelial cells, mesenchymal cells, or cancer cells, and a lipid composition containing the therapeutic agent and lipid nanoparticles (LNP).

### BACKGROUND ART

LNPs are materials that can deliver a therapeutic agent such as nucleic acids to the liver. An example of LNPs is a recently US Food and Drug Administration-approved short interfering RNA (siRNA) LNP therapy for transthyretin-mediated amyloidosis called Onpattro™. Despite these advances, it is currently impossible to predictably and rationally design nanoparticles for delivery to targeted tissues beyond the liver.

Conventionally, effective intracellular delivery materials have relied on an optimal balance of ionizable amines to bind and release RNAs (pKa between 6.0 and 6.5) and nanoparticle-stabilizing hydrophobicity. This exhaustive focus on ionizable cationic lipids has produced highly effective carriers for liver hepatocytes, but has not yielded effective carriers that are capable of reaching other organs.

Lipid nanoparticles (LNPs) are self-assembled nanostructures with the ability to encapsulate, protect, and deliver nucleic acids. Traditional LNPs are composed of ionizable cationic lipids, zwitterionic phospholipids, cholesterol and poly (ethylene glycol) (PEG) lipids. Screening and designing novel ionizable lipids have produced highly effective RNA delivery carriers for liver hepatocytes and mRNA vaccines. However, these efforts have not yielded effective carriers that can reach extrahepatic organs.

The present inventors have shown that incorporation of permanently cationic lipids (Non-patent documents 1 to 3) or LNP surface-modification with ligands enables RNA delivery to endothelial cells in the lung (Non-patent documents 4 to 7). However, cationic lipids are known to be toxic, and ligand-modification of LNPs is labor-intensive and potentially heterogeneous. Also, even with these technologies, RNA delivery has been still limited to the lung endothelial cells. Therefore, there is a demand for lipid nanoparticle compositions that don't use any constitutively cationic lipids or ligands.

There have been several studies on replacing cholesterol in LNP with cholesterol analogues. For example, oxidized cholesterols and cholesterol esters have been used to enhance RNA delivery efficiency to liver endothelial cells and all the cell types in the liver, respectively (Non-patent documents 8 and 9). Additionally, naturally-occurring cholesterol analogues such as phytosterols have been tested for improved endosomal escape efficiency in vitro (Non-patent document 10).

Several synthetic cholesterol analogues with basic functional group(s) have been developed for nucleic acid delivery carrier. DC-Cholesterol is a derivative of cholesterol with a tertiary amine group with pKa value of 7.8 (Non-patent documents 11 and 12). Conventionally, DC-cholesterol is formulated with a phospholipid, DOPE, to encapsulate nucleic acid as liposomes. Although combination of non-biodegradable ionizable lipids (C12-200 and cKK-E12) and DC-cholesterol was tested by our group in subcutaneous mRNA vaccines for enhanced mRNA delivery to dendritic cells in lymph node, it didn't show any advantage over cholesterol, or even reduced the delivery efficiency (Non-patent document 13).

### CITATION LIST

### Non Patent Literature

### [NPL 1]

Q. Cheng, T. Wei, L. Farbiak, L. T. Johnson, S. A. Dilliard, and D. J. Siegwart, “Selective organ targeting (SORT) nanoparticles for tissue-specific mRNA delivery and CRISPR-Cas gene editing,” Nat. Nanotechnol., vol. 15, no. April, 2020.

### [NPL 2]

L. M. Kranz et al., “Systemic RNA delivery to dendritic cells exploits antiviral defence for cancer immunotherapy,” Nature, vol. 534, no. 7607, pp. 396-401, 2016.

### [NPL 3]

Q. CHENG, T. Wei, and J. SIEGWART, Daniel, “COMPOSITIONS AND METHODS FOR ORGAN SPECIFIC DELIVERY OF NUCLEIC ACIDS,” WO 2020/051220, 2018.

### [NPL 4]

Q. Li et al., “Engineering Caveolae-Targeted Lipid Nanoparticles To Deliver mRNA to the Lungs,” ACS Chem. Biol., 2020.

### [NPL 5]

H. Parhiz et al., “PECAM-1 directed re-targeting of exogenous mRNA providing two orders of magnitude enhancement of vascular delivery and expression in lungs independent of apolipoprotein E-mediated uptake,” J. Control. Release, vol. 291, no. August, pp. 106-115, 2018.

### [NPL 6]

Y. Sakurai, T. Hada, A. Kato, Y. Hagino, W. Mizumura, and H. Harashima, “Effective Therapy Using a Liposomal siRNA that Targets the Tumor Vasculature in a Model Murine Breast Cancer with Lung Metastasis,” Mol. Ther.—Oncolytics, vol. 11, no. December, pp. 102-108, 2018.

### [NPL 7]

K. Kusumoto et al., “Lipid envelope-type nanoparticle incorporating a multifunctional peptide for systemic siRNA delivery to the pulmonary endothelium,” ACS Nano, vol. 7, no. 9, pp. 7534-7541, 2013.

### [NPL 8]

K. Paunovska et al., “Nanoparticles Containing Oxidized Cholesterol Deliver mRNA to the Liver Microenvironment at Clinically Relevant Doses,” vol. 1807748, pp. 1-7, 2019.

### [NPL 9]

K. Paunovska et al., “Analyzing 2000 in Vivo Drug Delivery Data Points Reveals Cholesterol Structure Impacts Nanoparticle Delivery,” ACS Nano, vol. 12, no. 8, pp. 8341-8349, 2018.

### [NPL 10]

S. Patel et al., “Naturally-occurring cholesterol analogues in lipid nanoparticles induce polymorphic shape and enhance intracellular delivery of mRNA,” Nat. Commun., vol. 11, no. 1, pp. 1-13, 2020.

### [NPL 11]

X. Gao and L. Huang, “A NOVEL CATIONIC LIPOSOME REAGENT FOR EFFICIENT TRANSFECTION OF MAMMALIAN CELLS,” Biochem. Biophys. Res. Commun., vol. 179, no. 1, pp. 280-285, 1991.

### [NPL 12]

D. Pozzi et al., “Mechanistic evaluation of the transfection barriers involved in lipid-mediated gene delivery: Interplay between nanostructure and composition,” Biochim. Biophys. Acta—Biomembr., vol. 1838, no. 3, pp. 957-967, 2014.

### [NPL 13]

M. A. Oberli et al., “Lipid Nanoparticle Assisted mRNA Delivery for Potent Cancer Immunotherapy,” Nano Lett., vol. 17, no. 3, pp. 1326-1335, 2017.

### SUMMARY OF INVENTION

### Technical Problem

In view of the above object in the background art, it is an object of the present invention to provide a lipid composition capable of delivering a therapeutic agent to endothelial cells, mesenchymal cells, or cancer cells in organs other than the liver.

The object to be solved by the present invention is to provide a method for delivering a therapeutic agent to endothelial cells, mesenchymal cells, or cancer cells, which can realize excellent delivery efficiency to organs other than the liver. Further, The object to be solved by the present invention is to provide a composition containing a therapeutic agent and lipid nanoparticles, which can realize excellent delivery efficiency to an organ other than the liver.

### Solution to Problem

As a result of diligent studies to solve the above objects, the present inventors have found that the use of an ionizable lipids and a cholesterol derivative having specific structure can achieve excellent delivery efficiency of the therapeutic agent to organs other than the liver. The present invention has been completed based on the above findings. According to the present invention, the following inventions are provided.

<1> A method for delivering a therapeutic agent to endothelial cells, mesenchymal cells, or cancer cells, which comprises administering a lipid composition to a subject, wherein the lipid composition comprises the therapeutic agent and lipid nanoparticle, and wherein the lipid nanoparticle comprises an ionizable lipid and a compound represented by formula (1) or a salt thereof.

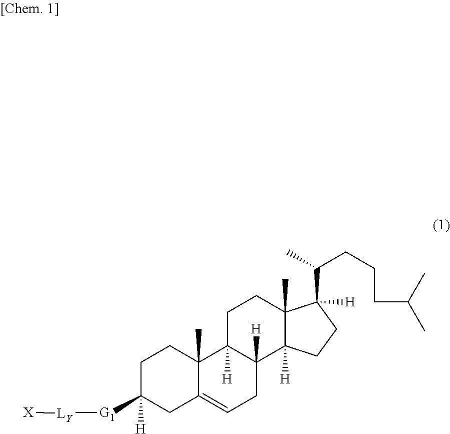

wherein G₁ represents —C(O)—, —OC(O)—, —O(CO)O— or —C(O)O—,
L_{Y} represents a single bond, an alkylene group having 1-14 carbon atoms, a substituted alkylene group having 1-14 carbon atoms, a heteroalkylene group having 1-14 carbon atoms, and a substituted heteroalkylene group having 1-14 carbon atoms.
X represents a basic functional group.

<2> The method of <1>, wherein the basic functional group represented by X is an amino group, a substituted amino group, a guanidino group, a 5 or 6 membered ring heterocyclic alkyl group, or a 5 or 6 membered ring heterocyclic aryl group.

<3> The method according to <1> or <2>, wherein the compound represented by formula (1) is a compound represented by formula (2)

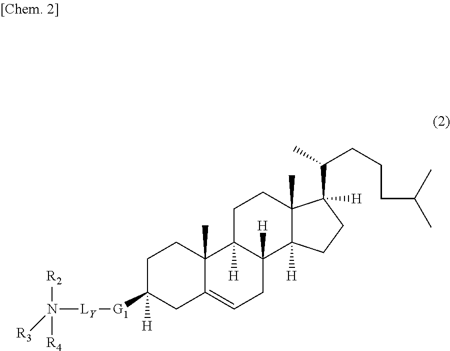

wherein G₁ represents —C(O)—, —OC(O)—, —O(CO)O— or —C(O)O—,
L_{Y} represents a single bond, an alkylene group having 1-14 carbon atoms, a substituted alkylene group having 1-14 carbon atoms, a heteroalkylene group having 1-14 carbon atoms, and a substituted heteroalkylene group having 1-14 carbon atoms.
R², R³ and R⁴ each independently represents a hydrogen atom, a hydrocarbon group having 1 to 4 carbon atoms which may be substituted with a hydroxyl group, or —C(NH₂)=N₂ and one of R², and
R³ and R⁴ may be absent.

<4> The method of <3>, wherein the compound represented by formula (2) is a compound represented by formula (3)

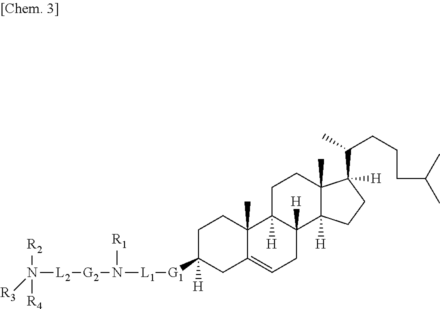

wherein G₁ represents —C(O)—, —OC(O)—, —O(CO)O— or —C(O)O—,
L₁ represents a single bond or an alkylene group having 1 to 6 carbon atoms,
R₁ represents a hydrogen atom, a hydrocarbon group containing 1 to 4 carbon atoms, or an aminoalkyl group having 1 to 4 carbon atoms,
G₂ represents a single bond, —C(O)—, —OC(O)—, —O(CO)O— or —C(O)O—,
L₂ represents an alkylene group having 1 to 6 carbons which may have an amino group,
R², R³ and R⁴ each independently represent a hydrogen atom, a hydrocarbon group having 1 to 4 carbon atoms which may be substituted with a hydroxyl group, or —C(NH₂)=NH₂, and one of R², R³ and R⁴ may be absent.

<5> The method of <4>, wherein the compound represented by formula (3) is a compound represented by formula (3A):

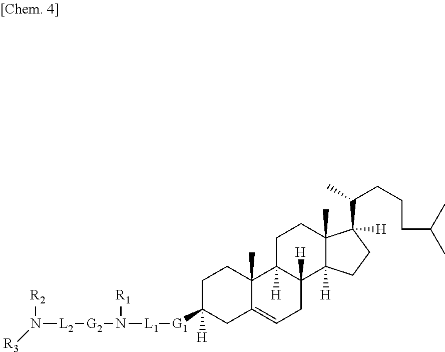

wherein G₁ represents —C(O)—, —OC(O)—, —O(CO)O— or —C(O)O—,
L₁ represents a single bond or an alkylene group having 1 to 6 carbon atoms,
R₁ represents a hydrogen atom, a hydrocarbon group having 1 to 4 carbon atoms, or an aminoalkyl group having 1 to 4 carbon atoms,
G₂ represents a single bond, —C(O)—, —OC(O)—, —O(CO)O— or —C(O)O—,
L₂ represents an alkylene group having 1 to 6 carbons which may have an amino group, and
R² and R³ is each independently a hydrogen atom, a hydrocarbon group having 1 to 4 carbon atoms which may be substituted with a hydroxyl group, or —C(NH₂)=NH₂.

<6> The method of any one of <1> to <5>, wherein G₁ represents —C(O)— or —C(O)O—.

<7> The method of <5> wherein R₁ represents a hydrogen atom or an aminoalkyl group having 1 to 4 carbon atoms.

<8> The method of <5>, wherein R₁ represents hydrogen atoms.

<9> The method of <5>, wherein G₂ represents a single bond or —C(O).

<10> The method of <5>, wherein G₂ represents a single binding.

<11> The method of <5>, wherein L₂ represents an alkylene group having 1 to 3 carbon atoms, and R² and R³ each independently represent a hydrogen atom or a hydrocarbon group having 1 to 2 carbon atoms which may be substituted with a hydroxyl group.

<12> The method according to <1> or <2>, wherein the compound represented by formula (1) or a salt thereof is any of the following.

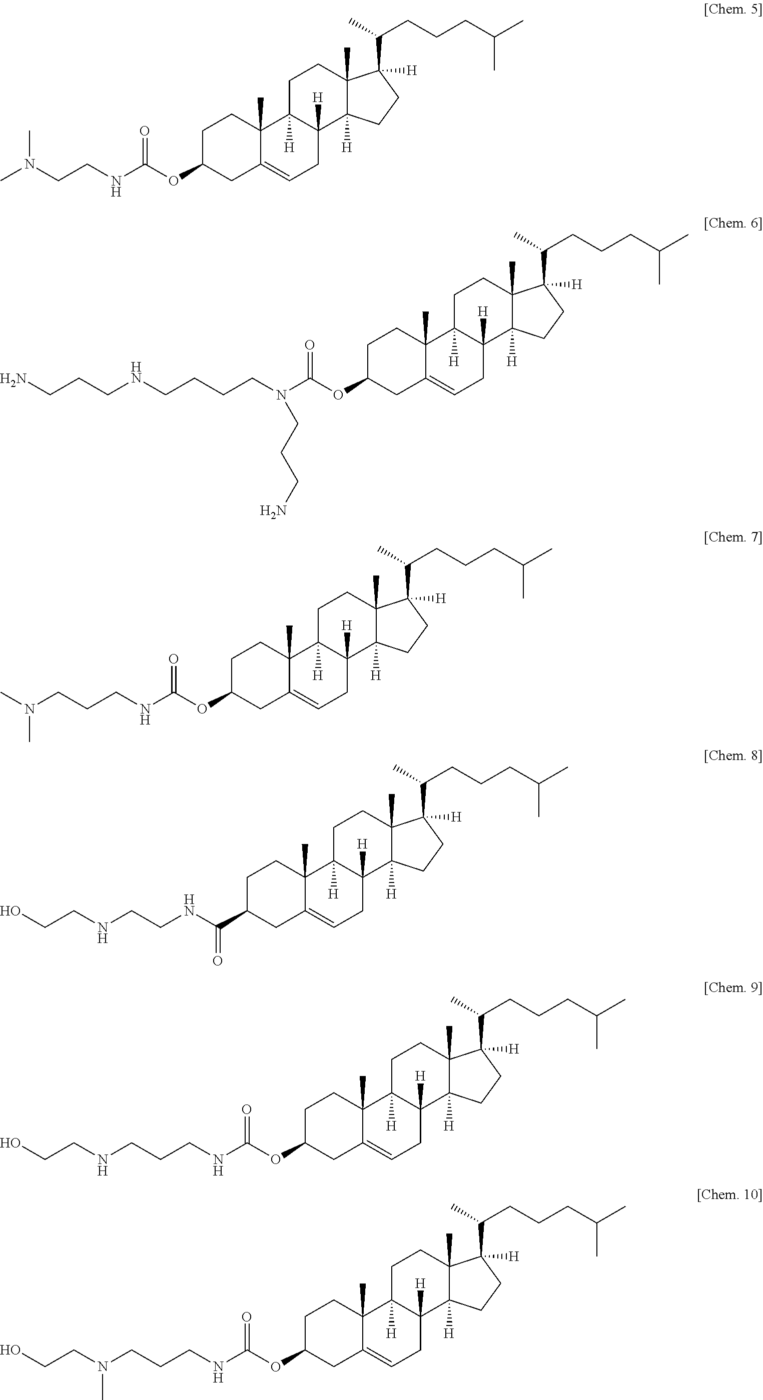

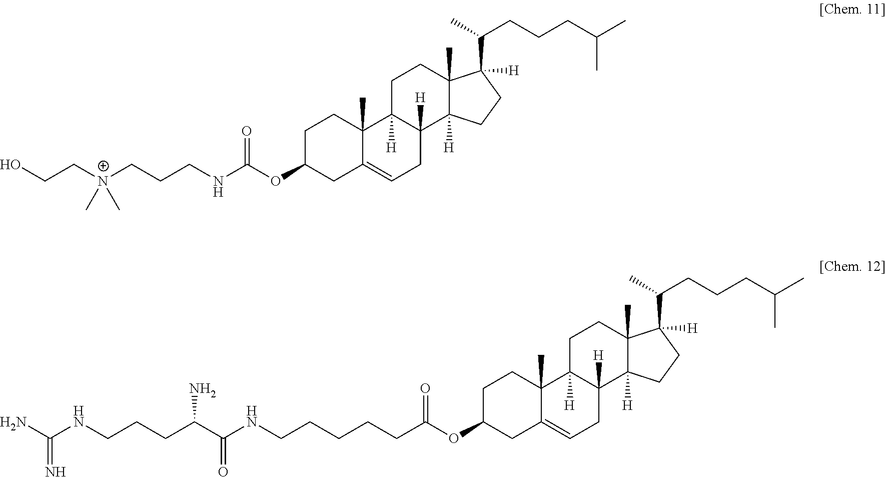

<13> The method of any one of <1> to <12>, wherein the content of the compound represented by formula (1) or a salt thereof is from 5 to 80 mol % based on the total lipid.

<14> The method of any one of <1> to <13>, wherein the therapeutic agent is a nucleic acid.

<15> The method of any one of <1> to <14>, wherein the therapeutic agent is DNA or RNA.

<16> The method of any one of <1> to <15>, wherein the therapeutic agent is mRNA or siRNA.

<17> The method of any one of <1> to <16>, wherein the ionizable lipid has at least one ionizable amino group and at least one biodegradable group, and wherein the biodegradable group is represented by —O(CO)O—, —O(CO)— or —(CO)O—.

<18> The method of any one of <1> to <17>, wherein the ionizable lipid is a compound represented by formula (4):

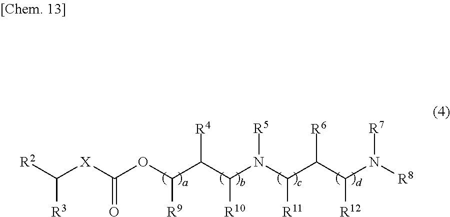

wherein X represents NR¹— or —O—,
R¹ represents a hydrogen atom, a hydrocarbon group having 6 to 24 carbon atoms, or a group represented by R²¹-L¹-R²²—, R²¹ represents a hydrocarbon group having 1 to 24 carbon atoms, and L¹ represents —O(CO)O—, —O(CO)—, —(CO)O—, —O—, or

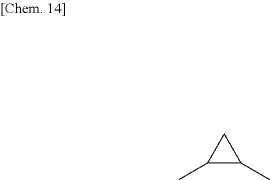

R²² is a divalent linking group and represents a hydrocarbon linking group having 1 to 18 carbon atoms,
R² and R³ each independently represent a hydrogen atom, a hydrocarbon group having 3 to 24 carbon atoms, or a group represented by R³¹-L²-R³²—, R³¹ represents a hydrocarbon group having 1 to 24 carbon atoms, and L² represents —O(CO)O—, —O(CO)—, —(CO)O—, —O—, or

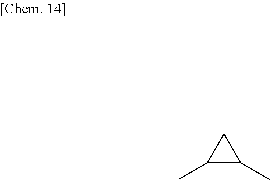

R³² is a divalent linking group and represents a hydrocarbon linking group having 1 to 18 carbon atoms,
each of R⁴, R⁵, R⁶, R⁷, R⁸, R⁹, R¹⁰, R¹¹, and R¹² independently represents a hydrogen atom or an optionally substituted alkyl group having 1-18 carbon atoms,
any one or more sets of R⁴ and R⁵, R¹⁰ and R⁵, R⁵ and R¹², R⁴ and R⁶, R⁵ and R⁶, R⁶ and R⁷, R⁶ and R¹⁰, R¹² and R⁷, and R⁷ and R⁸, may be linked together to form a 4- to 7-membered ring which may contain O atom,
a substituents on the optionally substituted alkyl group having 1-18 carbon atoms represents a hydroxyl groups, a carboxyl groups, an amino groups represented by NR⁴⁵R⁴⁶, a substituted or unsubstituted aryl group, a substituted or unsubstituted heteroaryl group, or a group represented by —O(CO)O—R⁴¹, —O(CO)—R⁴², —(CO)O—R⁴³, or —O—R⁴⁴, wherein R⁴¹, R⁴², R⁴³, R⁴⁴, R⁴⁵ and R⁴⁶ each independently represents a hydrocarbon group having 1 to 18 carbon atoms, a substituents on the substituted or unsubstituted aryl group and the substituted or unsubstituted heteroaryl group represents an alkyl groups having 1 to 18 carbon atoms, a hydroxyl groups, a carboxyl groups, an amino groups represented by —NR⁴⁵R⁴⁶, or a group represented by —O(CO)O—R⁴¹, —O(CO)—R⁴², —(CO)O—R⁴³, or —O—R⁴⁴, and R⁴¹, R⁴², R⁴³, R⁴⁴, R⁴⁵ and R⁴⁶ each independently represents a hydrocarbon group having 1 to 18 carbon atoms,
a, b, c, and d each independently represents an integer from 0 to 3, wherein a+b is 1 or more, and c+d is 1 or more.

<19> The method of any one of <1> to <17>, wherein the ionizable lipid is a compound represented by formula (1):

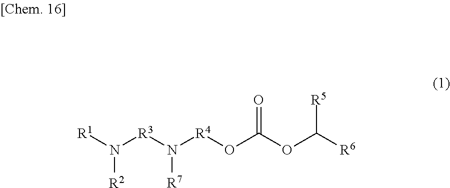

In the formula,
R¹ and R² each independently represent a hydrocarbon group having 1 to 18 carbon atoms, and R³ represents a hydrocarbon group having 2 to 8 carbon atoms, where the hydrocarbon groups represented by R¹, R², and R³ may be substituted with one or more substituents selected from —OH, COOH, —NR⁵¹R⁵², —OC(O)O—R⁵³, —C(O)O—R⁵⁴, —OC(O)—R⁵⁵, and —O—R⁵⁶,
R⁴ represents a hydrocarbon group having 1 to 8 carbon atoms,
R⁵ and R⁶ each independently represent a hydrocarbon group having 1 to 8 carbon atoms or —R⁸-L¹-R⁹, excluding a case that both R⁵ and R⁶ are hydrocarbon groups having 1 to 8 carbon atoms,
R⁷ represents —R¹⁰-L²-R¹¹-L³-R¹²,
R⁵¹ and R⁵² each independently represent a hydrocarbon group having 1 to 8 carbon atoms,
R⁵³, R⁵⁴, R⁵⁵, and R⁵⁶ each independently represent a hydrocarbon group having 1 to 24 carbon atoms,
the hydrocarbon groups represented by R⁵³, R⁵⁴, R⁵⁵, and R⁵⁶ may be substituted with an aryl group having 6 to 20 carbon atoms or —S—R⁵⁸,
the above-described aryl group having 6 to 20 carbon atoms may be substituted with —OH, COOH, —NR⁵¹R⁵², —OC(O)O—R⁵³, —C(O)O—R⁵⁴, —OC(O)—R⁵⁵, —O—R⁵⁶, or -(hydrocarbon group having 1 to 12 carbon atoms)-R⁵⁷,
R⁵⁸ represents a hydrocarbon group having 1 to 12 carbon atoms, and
R⁵⁷ represents —OH, COOH, —NR⁶¹R⁶², —OC(O)O—R⁶³, —C(O)O—R⁶⁴, —OC(O)—R⁶⁵, or —O—R⁶⁶,
R⁶¹ and R⁶² each independently represent a hydrocarbon group having 1 to 8 carbon atoms,
R⁶³, R⁶⁴, R⁶⁵, and R⁶⁶ each independently represent a hydrocarbon group having 1 to 24 carbon atoms,
the hydrocarbon groups represented by R⁶³, R⁶⁴, R⁶⁵, and R⁶⁶ may be substituted with an aryl group having 6 to 20 carbon atoms or —S—R⁶⁸,
the above-described aryl group having 6 to 20 carbon atoms may be substituted with —OH, COOH, —NR⁶¹R⁶², —OC(O)O—R⁶³, —C(O)O—R⁶⁴, —OC(O)—R⁶⁵, —O—R⁶⁶, or -(hydrocarbon group having 1 to 12 carbon atoms)-R⁶⁷,
R⁶⁸ represents a hydrocarbon group having 1 to 12 carbon atoms, and
L¹, L², and L³ each independently represent —OC(O)O—, —C(O)O—, —OC(O)—, or —O—.
R⁸ represents a hydrocarbon group having 1 to 12 carbon atoms,
R⁹ represents a hydrocarbon group having 1 to 24 carbon atoms,
R¹⁰ represents a hydrocarbon group having 1 to 8 carbon atoms,
R¹¹ represents a hydrocarbon group having 1 to 24 carbon atoms,
R¹² represents a hydrocarbon group having 1 to 24 carbon atoms,
the hydrocarbon groups represented by R⁹ and R¹² may be substituted with an aryl group, —OC(O)O—R⁵³, —C(O)O—R⁵⁴, —OC(O)—R⁵⁵, or —S—R⁵⁸, where definitions of R⁵³, R⁵⁴, R⁵⁵, and R⁵⁸ are as described above, and
the hydrocarbon group represented by R¹¹ may be substituted with —OC(O)O—R⁵³, —C(O)O—R⁵⁴, or —OC(O)—R⁵⁵, where the definitions of R⁵³, R⁵⁴, and R⁵⁵ are as described above.

<20> The method of any one of <1> to <17>, wherein the ionizable lipid is a compound represented by the following formula (5):

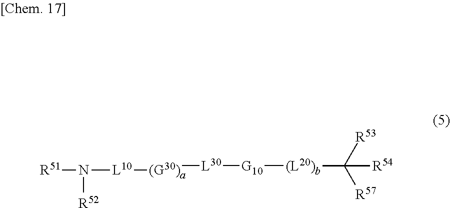

wherein R⁵¹ and R⁵² each independently represent a hydrocarbon group having 1 to 21 carbon atoms which may have a substituent A,
the substituent A represents a hydroxyl group, or a group represented by -G²⁰-CH(R⁵⁵)(R⁵⁶), —N(R⁵⁸)(R⁵⁹) or -G²⁰-R⁶⁰,
G²⁰ represents —O(CO)—, or —(CO)O—,
R⁵⁵ and R⁵⁶ each independently represent a hydrogen atom or a hydrocarbon group having 1 to 18 carbon atoms,
R⁵⁸ and R⁵⁹ each independently represent a hydrogen atom or a cyclic hydrocarbon group having 3 to 6 carbon atoms which may have a substituent B,
the substituent B is —N(R⁶¹)(R⁶²),
R⁶¹ and R⁶² each independently represent a hydrogen atom or a hydrocarbon group having 1 to 6 carbon atoms,
R⁶⁰ represents a hydrocarbon group having 1 to 18 carbon atoms,
L¹⁰ represents a hydrocarbon group having 1 to 18 carbon atoms, G³⁰ indicates —S—(CO)—NR⁶⁴,
R⁶⁴ represents a group represented by -L³⁰-G²⁰-CH(R⁵⁵)(R⁵⁶) a represents 0 or 1,
L³⁰ represents a single bond or a hydrocarbon group having 1 to 18 carbon atoms,
G¹⁰ represents —O(CO)—, —(CO)O—, —O(CO)O—, or —N(C(O)R⁶³)—,
R⁶³ represents a hydrocarbon group having 1 to 18 carbon atoms,
L²⁰ represents a hydrocarbon group having 1 to 6 carbon atoms,
b represents 0 or 1,
R⁵³, R⁵⁴ and R⁵⁷ each independently represent a hydrogen atom or a hydrocarbon group having 1 to 21 carbon atoms which may have a substituent C,
the substituent C represents a group represented by —(CO)O R⁶⁵ or —O(CO)—R⁶⁵,
R⁶⁵ represents a hydrocarbon group having 1 to 18 carbon atoms or a group represented by -L⁴⁰-CH(R⁶⁶)(R⁶⁷),
L⁴⁰ represents a hydrocarbon group having 1 to 6 carbon atoms,
R⁶⁶ and R⁶⁷ represent a hydrocarbon group having 1 to 10 carbon atoms or an alkoxy group.

<21> The method of any one of <1> to <20>, wherein the lipid nanoparticle further comprises a lipid having a nonionic hydrophilic polymer.

<22> The method of any one of <1> to <21>, wherein the lipid nanoparticle further comprises a phospholipid.

<23> The method according to any one of <1> to <22>, wherein the lipid composition is administered to a subject by intravenous or intramuscular injection.

<24> The method of any one of <1> to <23>, wherein the mesenchymal cell is myocyte.

<25> The method of any one of <1> to <23>, wherein the mesenchymal cell is extracellular matrix producing cell.

<26> The method of any one of <1> to <23>, wherein the extracellular matrix cell is stellate cell or fibroblast.

<27> The method of any one of <1> to <23>, wherein the stellate cell is hepatic stellate cell, pancreatic stellate cell, or colonic stellate cell.

<28> A lipid composition comprising a therapeutic agent and lipid nanoparticles, wherein the lipid nanoparticle comprises a compound represented by formula (1) or a salt thereof and an ionizable lipid having a biodegradable group,

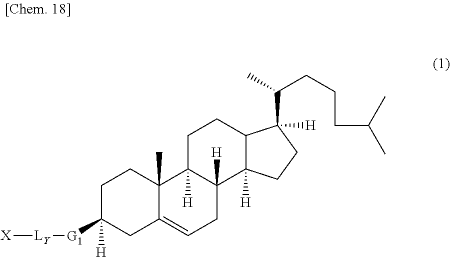

wherein G₁ represents —C(O)—, —OC(O)—, —O(CO)O— or —C(O)O—,
L_{Y} represents a single bond, an alkylene group having 1-14 carbon atoms, a substituted alkylene group having 1-14 carbon atoms, a heteroalkylene group having 1-14 carbon atoms, and a substituted heteroalkylene group having 1-14 carbon atoms,
X represents a basic functional group.

<29> The lipid composition of <28>, wherein the basic functional group represented by X is an amino group, a substituted amino group, a guanidino group, a 5-6 membered ring heterocyclic alkyl group, or a 5-6 membered ring heterocyclic aryl group.

<30> The lipid composition of <28> or <29>, wherein the compound represented by formula (1) is a compound represented by formula (2)

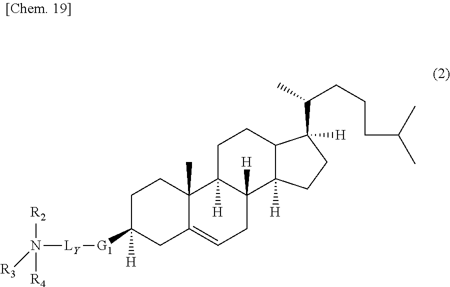

wherein G₁ represents —C(O)—, —OC(O)—, —O(CO)O— or —C(O)O—,
L_{Y} represents a single bond, an alkylene group having 1-14 carbon atoms, a substituted alkylene group having 1-14 carbon atoms, a heteroalkylene group having 1-14 carbon atoms, and a substituted heteroalkylene group having 1-14 carbon atoms.
R², R³ and R⁴ each independently represents a hydrogen atom, a hydrocarbon group having 1 to 4 carbon atoms which may be substituted with a hydroxyl group, or —C(NH₂)=NH₂ and one of R², and
R³ and R⁴ may be absent.

<31> The lipid composition according to <30>, wherein the compound represented by formula (2) is a compound represented by formula (3)

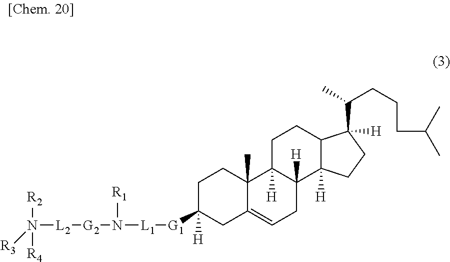

wherein G₁ represents —C(O)—, —OC(O)—, —O(CO)O— or —C(O)O—,
L₁ represents a single bond or an alkylene group having 1 to 6 carbon atoms,
R₁ represents a hydrogen atom, a hydrocarbon group containing 1 to 4 carbon atoms, or an aminoalkyl group having 1 to 4 carbon atoms,
G₂ represents a single bond, —C(O)—, —OC(O)—, —O(CO)O— or —C(O)O—,
L₂ represents an alkylene group having 1 to 6 carbons which may have an amino group,
R², R³ and R⁴ each independently represent a hydrogen atom, a hydrocarbon group having 1 to 4 carbon atoms which may be substituted with a hydroxyl group, or —C(NH₂)=NH₂, and one of R², R³ and R⁴ may be absent.

<32> The lipid composition according to any one of <28> to <31>, wherein the ionizable lipid having a biodegradable group is a compound represented by formula (4)

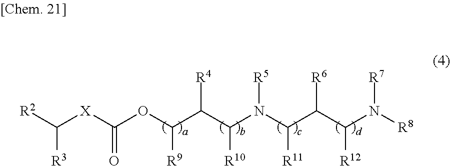

wherein X represents NR¹— or —O—,
R¹ represents a hydrogen atom, a hydrocarbon group having 6 to 24 carbon atoms, or a group represented by R²¹-L¹-R²²—, R²¹ represents a hydrocarbon group having 1 to 24 carbon atoms, and L¹ represents —O(CO)O—, —O(CO)—, —(CO)O—, —O—, or

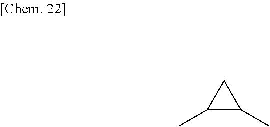

R²² is a divalent linking group and represents a hydrocarbon linking group having 1 to 18 carbon atoms,
R² and R³ each independently represent a hydrogen atom, a hydrocarbon group having 3 to 24 carbon atoms, or a group represented by R³¹-L²-R³²—, R³¹ represents a hydrocarbon group having 1 to 24 carbon atoms, and L² represents —O(CO)O—, —O(CO)—, —(CO)O—, —O—, or

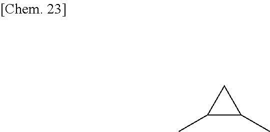

R³² is a divalent linking group and represents a hydrocarbon linking group having 1 to 18 carbon atoms,
each of R⁴, R⁵, R⁶, R⁷, R⁸, R⁹, R¹⁰, R¹¹, and R¹² independently represents a hydrogen atom or an optionally substituted alkyl group having 1-18 carbon atoms,
any one or more sets of R⁴ and R⁵, R¹⁰ and R⁵, R⁵ and R¹², R⁴ and R⁶, R⁵ and R⁶, R⁶ and R⁷, R⁶ and R¹⁰, R¹² and R⁷, and R⁷ and R⁸, may be linked together to form a 4- to 7-membered ring which may contain O atom,
a substituents on the optionally substituted alkyl group having 1-18 carbon atoms represents a hydroxyl groups, a carboxyl groups, an amino groups represented by NR⁴⁵R⁴⁶, a substituted or unsubstituted aryl group, a substituted or unsubstituted heteroaryl group, or a group represented by —O(CO)O—R⁴¹, —O(CO)—R⁴², —(CO)O—R⁴³, or —O—R⁴⁴, wherein R⁴¹, R⁴², R⁴³, R⁴⁴, R⁴⁵ and R⁴⁶ each independently represents a hydrocarbon group having 1 to 18 carbon atoms,
a substituents on the substituted or unsubstituted aryl group and the substituted or unsubstituted heteroaryl group represents an alkyl groups having 1 to 18 carbon atoms, a hydroxyl groups, a carboxyl groups, an amino groups represented by —NR⁴⁵R⁴⁶, or a group represented by —O(CO)O—R⁴¹, —O(CO)—R⁴², —(CO)O—R⁴³, or —O—R⁴⁴, and R⁴¹, R⁴², R⁴³, R⁴⁴, R⁴⁵ and R⁴⁶ each independently represents a hydrocarbon group having 1 to 18 carbon atoms,
a, b, c, and d each independently represents an integer from 0 to 3, wherein a+b is 1 or more, and c+d is 1 or more.

<33> The lipid composition according to any one of <28> to <31>, wherein the ionizable lipid is a compound represented by formula (1):

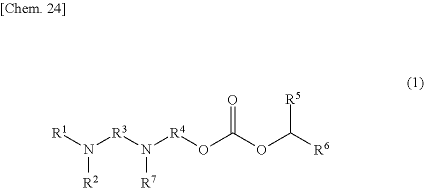

In the formula,
R¹ and R² each independently represent a hydrocarbon group having 1 to 18 carbon atoms, and R³ represents a hydrocarbon group having 2 to 8 carbon atoms, where the hydrocarbon groups represented by R¹, R², and R³ may be substituted with one or more substituents selected from —OH, COOH, —NR⁵¹R⁵², —OC(O)O—R⁵³, —C(O)O—R⁵⁴, —OC(O)—R⁵⁵, and —O—R⁵⁶,
R⁴ represents a hydrocarbon group having 1 to 8 carbon atoms,
R⁵ and R⁶ each independently represent a hydrocarbon group having 1 to 8 carbon atoms or —R⁸-L¹-R⁹, excluding a case that both R⁵ and R⁶ are hydrocarbon groups having 1 to 8 carbon atoms,
R⁷ represents —R¹⁰-L²-R¹¹-L³-R¹²,
R⁵¹ and R⁵² each independently represent a hydrocarbon group having 1 to 8 carbon atoms,
R⁵³, R⁵⁴, R⁵⁵, and R⁵⁶ each independently represent a hydrocarbon group having 1 to 24 carbon atoms,
the hydrocarbon groups represented by R⁵³, R⁵⁴, R⁵⁵, and R⁵⁶ may be substituted with an aryl group having 6 to 20 carbon atoms or —S—R⁵⁸,
the above-described aryl group having 6 to 20 carbon atoms may be substituted with —OH, COOH, —NR⁵¹R⁵², —OC(O)O—R⁵³, —C(O)O—R⁵⁴, —OC(O)—R⁵⁵, —O—R⁵⁶, or -(hydrocarbon group having 1 to 12 carbon atoms)-R⁵⁷,
R⁵⁸ represents a hydrocarbon group having 1 to 12 carbon atoms, and
R⁵⁷ represents —OH, COOH, —NR⁶¹R⁶², —OC(O)O—R⁶³, —C(O)O—R⁶⁴, —OC(O)—R⁶⁵, or —O—R⁶⁶.
R⁶¹ and R⁶² each independently represent a hydrocarbon group having 1 to 8 carbon atoms,
R⁶³, R⁶⁴, R⁶⁵, and R⁶⁶ each independently represent a hydrocarbon group having 1 to 24 carbon atoms,
the hydrocarbon groups represented by R⁶³, R⁶⁴, R⁶⁵, and R⁶⁶ may be substituted with an aryl group having 6 to 20 carbon atoms or —S—R⁶⁸,
the above-described aryl group having 6 to 20 carbon atoms may be substituted with —OH, COOH, —NR⁶¹R⁶², —OC(O)O—R⁶³, —C(O)O—R⁶⁴, —OC(O)—R⁶⁵, —O—R⁶⁶, or -(hydrocarbon group having 1 to 12 carbon atoms)-R⁶⁷,
R⁶⁸ represents a hydrocarbon group having 1 to 12 carbon atoms, and
L¹, L², and L³ each independently represent —OC(O)O—, —C(O)O—, —OC(O)—, or —O—.
R⁸ represents a hydrocarbon group having 1 to 12 carbon atoms,
R⁹ represents a hydrocarbon group having 1 to 24 carbon atoms,
R¹⁰ represents a hydrocarbon group having 1 to 8 carbon atoms,
R¹¹ represents a hydrocarbon group having 1 to 24 carbon atoms,
R¹² represents a hydrocarbon group having 1 to 24 carbon atoms,
the hydrocarbon groups represented by R⁹ and R¹² may be substituted with an aryl group, —OC(O)O—R⁵³, —C(O)O—R⁵⁴, —OC(O)—R⁵⁵, or —S—R⁵⁸, where definitions of R⁵³, R⁵⁴, R⁵⁵, and R⁵⁸ are as described above, and
the hydrocarbon group represented by R¹¹ may be substituted with —OC(O)O—R⁵³, —C(O)O—R⁵⁴, or —OC(O)—R⁵⁵, where the definitions of R⁵³, R⁵⁴, and R⁵⁵ are as described above.

<34> The lipid composition of any one of <28> to <31>, wherein the ionizable lipid having a biodegradable group is a compound represented by Formula (5):

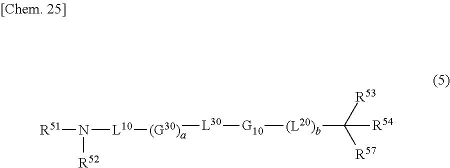

wherein R⁵¹ and R⁵² each independently represent a hydrocarbon group having 1 to 21 carbon atoms which may have a substituent A,
the substituent A represents a hydroxyl group, or a group represented by -G²⁰-CH(R⁵⁵)(R⁵⁶), —N(R⁵⁸)(R⁵⁹) or -G²⁰-R⁶⁰,
G²⁰ represents —O(CO)—, or —(CO)O—,
R⁵⁵ and R⁵⁶ each independently represent a hydrogen atom or a hydrocarbon group having 1 to 18 carbon atoms,
R⁵⁸ and R⁵⁹ each independently represent a hydrogen atom or a cyclic hydrocarbon group having 3 to 6 carbon atoms which may have a substituent B,
the substituent B is —N(R⁶¹)(R⁶²),
R⁶¹ and R⁶² each independently represent a hydrogen atom or a hydrocarbon group having 1 to 6 carbon atoms,
R⁶⁰ represents a hydrocarbon group having 1 to 18 carbon atoms,
L¹⁰ represents a hydrocarbon group having 1 to 18 carbon atoms, G³⁰ indicates —S—(CO)—NR⁶⁴,
R⁶⁴ represents a group represented by -L³⁰-G²⁰-CH(R⁵⁵)(R⁵⁶) a represents 0 or 1,
L³⁰ represents a single bond or a hydrocarbon group having 1 to 18 carbon atoms,
G¹⁰ represents —O(CO)—, —(CO)O—, —O(CO)O—, or —N(C(O)R⁶³)—,
R⁶³ represents a hydrocarbon group having 1 to 18 carbon atoms,
L²⁰ represents a hydrocarbon group having 1 to 6 carbon atoms,
b represents 0 or 1,
R⁵³, R⁵⁴ and R⁵⁷ each independently represent a hydrogen atom or a hydrocarbon group having 1 to 21 carbon atoms which may have a substituent C,
the substituent C represents a group represented by —(CO)O R⁶⁵ or —O(CO)—R⁶⁵,
R⁶⁵ represents a hydrocarbon group having 1 to 18 carbon atoms or a group represented by -L⁴⁰-CH(R⁶⁶)(R⁶⁷),
L⁴⁰ represents a hydrocarbon group having 1 to 6 carbon atoms,
R⁶⁶ and R⁶⁷ represent a hydrocarbon group having 1 to 10 carbon atoms or an alkoxy group.

<35> The lipid composition of any one of <28> to <34>, wherein the content of the compound represented by Formula (1) or a salt thereof is from 5 to 80 mol % based on the total lipid.

<36> The lipid composition of any one of <28> to <35>, wherein the therapeutic agent is a nucleic acid.

<37> The lipid composition of any one of <28> to <36>, wherein the therapeutic agent is DNA or RNA.

<38> The lipid composition of any one of <28> to <37>, wherein the treatment agent is mRNA or siRNA.

### Advantageous Effects of Invention

According to the method and composition of the present invention, excellent delivery efficiency of a therapeutic agent can be realized even for organs other than the liver.

### BRIEF DESCRIPTION OF DRAWINGS

FIG. 1 shows the results of measuring the uptake of lipid compositions into various tissues.

FIG. 2 shows the results of measuring the uptake of lipid compositions into various tissues.

FIG. 3 shows the results of measuring the uptake of lipid compositions into various tissues.

FIG. 4 shows the results of measuring the uptake of lipid compositions into various tissues.

FIG. 5 shows the results of measuring the uptake of lipid compositions into various tissues.

FIG. 6 shows the results of measuring the uptake of lipid compositions into various tissues.

FIG. 7 shows the results of In vivo endothelial cell RNA delivery with various ionizable lipid.

FIG. 8 shows the results of In vivo endothelial cell RNA delivery with various ionizable cholesterol ratio (1) HAPC-Cholesterol.

FIG. 9 shows the results of In vivo endothelial cell RNA delivery with various ionizable cholesterol ratio (2) DC-cholesterol.

FIG. 10 shows the results of In vivo endothelial cell RNA delivery with various ionizable cholesterol ratio (3) HAPC-Cholesterol.

FIG. 11 shows the results of serum protein-independent RNA delivery to endothelial cells.

FIG. 12 shows the results of LNP-mediated endothelial cell damage analysis in vitro.

FIG. 13 shows the results of In vitro hemocompatibility analysis using human primary red blood cells.

FIG. 14 shows the results of In vivo hepatic stellate cell delivery.

FIG. 15 shows the results of In vivo RNA delivery to extrahepatic extracellular matrix producing cells.

FIG. 16 shows the results of In vivo RNA delivery to extrahepatic extracellular matrix producing cells.

FIG. 17 shows the results of intramuscular administration and delivery to myocytes and endothelial cells.

FIG. 18 shows the results of intramuscular administration and delivery to myocytes and endothelial cells.

FIG. 19 shows the results of In vitro delivery to cancer cell.

### DESCRIPTION OF EMBODIMENTS

Hereinafter, the present invention will be described in detail.

In this specification, “˜” denotes a range including a numerical value described before and after it as a minimum value and a maximum value, respectively.

The present invention relates to a method for delivering a therapeutic agent to endothelial cells, mesenchymal cells, or cancer cells, which comprises administering a lipid composition to a subject,

wherein the lipid composition comprises the therapeutic agent and lipid nanoparticle, and wherein the lipid nanoparticle comprises an ionizable lipid and a compound represented by formula (1) or a salt thereof.

The present invention relate to a lipid composition comprising a therapeutic agent and lipid nanoparticles, wherein the lipid nanoparticle comprises a compound represented by formula (1) or a salt thereof and an ionizable lipid having a biodegradable group,

In the present invention, it has been found that cell-type and tissue-type tropism can be converted from liver hepatocytes to endothelial cells, mesenchymal cells, or cancer cells in various organs by replacing cholesterol with its analogues. The combination of ionizable lipid and cholesterol analogue which is a compound represented by formula (1) is useful for safe and efficacious delivery of a therapeutic agent for systemic and local delivery for endothelial cells, mesenchymal cells, or cancer cells.

Examples of mesenchymal cell include bone cells (osteoblasts), cartilage cells (chondrocytes), muscle cells (myocytes, skeletal myocytes, cardiac myocytes), connective cells (fibroblasts, myofibroblasts, stellate cells), marrow stromal cells, tenocyte, and fat cells (adipocytes),

Fibroblasts are ubiquitous mesenchymal cells that are normally found in the stroma of many tissues.

Myofibroblast is an activated form of fibroblast that is capable of contraction due to the presence of cytoskeletal proteins that are normally found in smooth-muscle cells—in particular, α-smooth muscle actin.

Stellate cell is a fibroblast that store retinoids and found at various organs such as the liver, pancreas, lung, kidney, intestine, spleen, adrenal gland, ductus deferens and vocal cords.

### <Compound Represented by Formula (1), Formula (2) or Formula (3)>

In the present invention, the compound represented by the following formula (1) or a salt thereof is used.

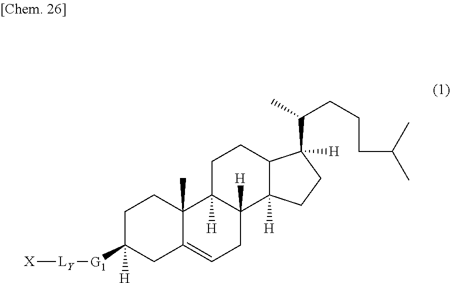

wherein G₁ represents —C(O)—, —OC(O)—, —O(CO)O— or —C(O)O—,
L_{Y} represents a single bond, an alkylene group having 1-14 carbon atoms, a substituted alkylene group having 1-14 carbon atoms, a heteroalkylene group having 1-14 carbon atoms, and a substituted heteroalkylene group having 1-14 carbon atoms.
X represents a basic functional group.

Preferably, G₁ represents —C(O)—, —OC(O)—, —O(CO)O— or —C(O)O—. The alkylene group having 1 to 14 carbon atoms represented by L_{Y} may be linear or branched, and may be chain-like or cyclic, and may have 1 to 12 carbon atoms, more preferably 1 to 10 carbon atoms, and still more preferably 2 to 10 carbon atoms. Specific examples thereof include methylene group, ethylene group, trimethylene group, tetramethylene group, pentamethylene group, hexamethylene group, heptamethylene group, octamethylene group, nonamethylene group, decamethylene group, undecamethylene group, dodecamethylene group and the like.

Examples of the alkylene group having 1-14 carbon atoms in the substituted alkylene group having 1-14 carbon atoms represented by L_{Y} are as described above. Examples of the substituent contained in the alkylene group having 1 to 14 carbon atoms include a hydrocarbon group having 1 to 4 carbon atoms, an aminoalkyl group having 1 to 4 carbon atoms, and an amino group.

The heteroalkylene group having 1-14 carbon atoms represented by L_{Y} is a group in which one or more heteroatoms selected from an oxygen atom, a nitrogen atom or a sulfur atom are linked to an alkylene group having 1-14 carbon atoms, or a group in which one or more heteroatoms selected from an oxygen atom, a nitrogen atom or a sulfur atom are present in the alkyl chain of an alkylene group having 1 to 14 carbon atoms. As Examples thereof, the heteroalkylene group contains —O—, —S—, —NH—, —NR—, —C(O)—, —CN—, —NR—C(O)—, —C(O)O—, —OC(O)—, —OC(O)O— and the like in the alkyl group, but the examples are not limited thereto. Further, two heteroatoms may be continuous, such as —S—S—.

In the substituted heteroalkylene groups having 1 to 14 carbon atoms represented by L_{Y}, examples of the substituents on the alkylene group having 1 to 14 carbon atoms include a hydrocarbon group having 1 to 4 carbon atoms, an aminoalkyl group having 1 to 4 carbon atoms, and an amino group.
L_{Y} is preferably a heteroalkylene group having 1-14 carbon atoms or a substituted heteroalkylene group having 1-14 carbon atoms.

Examples of the basic functional group represented by X include amino group, substituted amino group, guanidino group, 5- or 6-membered heterocyclic alkyl group, or 5- or 6-membered heterocyclic aryl group. Examples of the substituent on the substituted amino group include an alkyl group having 1 to 4 carbon atoms which may be substituted, —C(NH₂) ═NH₂ and the like.

Examples of the heterocyclic alkyl group include a cyclic functional group containing one or more heteroatoms selected from an oxygen atom, a nitrogen atom or a sulfur atom, and a carbon atom. Examples include, but are not limited to, pyrrolidine group, pyrazolidine group, imidazolidine group, pyrroline group, pyrazoline group, imizoline group, piperidine group, piperazine group, and morpholine group.

Examples of the heterocyclic aryl group include an aromatic ring containing one or more heteroatoms selected from an oxygen atom, a nitrogen atom or a sulfur atom, and a carbon atom. Examples include, but are not limited to, pyrrol group, imidazole group, pyrazole group, oxazole group, isoxazole group, thiazole group, isothiazole group, pyridine group, pyrazine group, pyrimidine group, pyridazine group, triazine group, tetrazine group, pentazine group and the like.

The compound represented by the formula (1) may be preferably a compound represented by the formula (2).

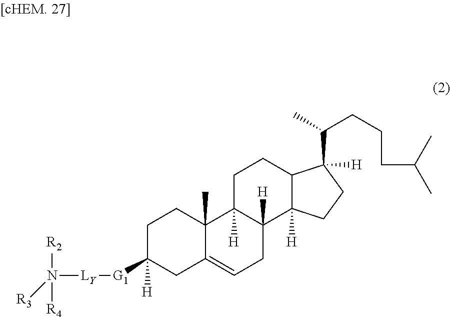

wherein G₁ represents —C(O)—, —OC(O)—, —O(CO)O— or —C(O)O—,
L_{Y} represents a single bond, an alkylene group having 1-14 carbon atoms, a substituted alkylene group having 1-14 carbon atoms, a heteroalkylene group having 1-14 carbon atoms, and a substituted heteroalkylene group having 1-14 carbon atoms.
R², R³ and R⁴ each independently represents a hydrogen atom, a hydrocarbon group having 1 to 4 carbon atoms which may be substituted with a hydroxyl group, or —C(NH₂)=NH₂ and one of R², and
R³ and R⁴ may be absent.

The definitions of G₁ and L_{Y} in the formula (2) are synonymous with the definitions in the formula (1).

Examples of the hydrocarbon group having 1 to 4 carbon atoms in the hydrocarbon group having 1 to 4 carbon atoms which may be substituted with a hydroxyl group, which is represented by R², R³ or R⁴, include methyl group, ethyl group, n-propy group 1, isopropyl group, n-butyl group, iso-butyl group, tert-butyl group and the like.
R², R³ and R⁴ are preferably hydrogen atom, methyl group, 1-hydroxyethyl group, or —C (NH₂)=NH₂.

The compound represented by the formula (2) may be preferably a compound represented by the formula (3).

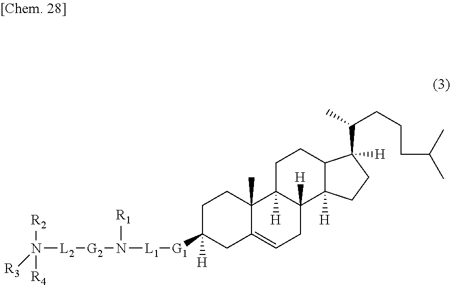

wherein G₁ represents —C(O)—, —OC(O)—, —O(CO)O— or —C(O)O—,
L₁ represents a single bond or an alkylene group having 1 to 6 carbon atoms,
R₁ represents a hydrogen atom, a hydrocarbon group containing 1 to 4 carbon atoms, or an aminoalkyl group having 1 to 4 carbon atoms,
G₂ represents a single bond, —C(O)—, —OC(O)—, —O(CO)O— or —C(O)O—,
L₂ represents an alkylene group having 1 to 6 carbons which may have an amino group,
R², R³ and R⁴ each independently represent a hydrogen atom, a hydrocarbon group having 1 to 4 carbon atoms which may be substituted with a hydroxyl group, or —C(NH₂)=N₂, and one of R², R³ and R⁴ may be absent.

The definitions of G¹, R², R³ and R⁴ in the formula (3) are synonymous with the definitions in the formula (1) and the formula (2).

Examples of the alkylene group having 1 to 6 carbon atoms represented by L₁ include methylene group, ethylene group, propane-1,3-diyl group, butane-1,4-diyl group, pentane-1,5-diyl group, hexane-1,6-diyl group and the like. L1 is preferably a single bond or hexane-1,6-diyl group.

The compound represented by the formula (3) may be preferably a compound represented by the formula (3A).

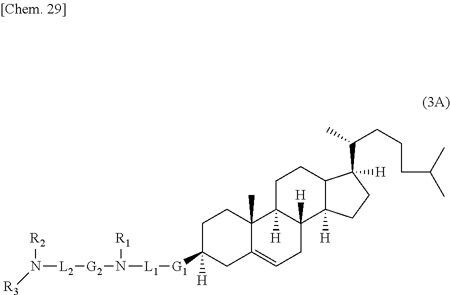

wherein G₁ represents —C(O)—, —OC(O)—, —O(CO)O— or —C(O)O—,
L₁ represents a single bond or an alkylene group having 1 to 6 carbon atoms,
R₁ represents a hydrogen atom, a hydrocarbon group having 1 to 4 carbon atoms, or an aminoalkyl group having 1 to 4 carbon atoms,
G₂ represents a single bond, —C(O)—, —OC(O)—, —O(CO)O— or —C(O)O—,
L₂ represents an alkylene group having 1 to 6 carbons which may have an amino group, and
R² and R³ is each independently a hydrogen atom, a hydrocarbon group having 1 to 4 carbon atoms which may be substituted with a hydroxyl group, or —C(NH₂)=NH₂.

Examples of the hydrocarbon group having 1 to 4 carbon atoms represented by R¹ include methyl group, ethyl group, propyl group, butyl group and the like.

Examples of aminoalkyl having 1 to 4 carbon atoms represented by R¹ include aminomethyl group, aminoethyl group, aminopropyl group, and aminobutyl group.
R¹ is preferably a hydrogen atom or an aminoalkyl group having 1 to 4 carbon atoms.
G₂ preferably represents a single bond or —C(O).

Examples of the alkylene groups having 1 to 6 carbon atoms in the the alkylene groups having 1 to 6 carbon atoms which may have the amino group, represented by L₂, include methylene group, ethylene group, propane-1,3-diyl group, butane-1,4-diyl group, pentane-1,5-diyl group, hexane-1,6-diyl group and the like.
L₂ is preferably ethylene group, propane-1,3-diyl group, or butane-1,4-diyl group having an amino group.

Example of the salt of the compound represented by formula (1), formula (2), formula (3) or formula (3A) include a salt in a basic group, and include salts with mineral acids such as hydrochloric acid, hydrobromic acid, nitric acid, and sulfuric acid; salts with organic carboxylic acids such as formic acid, acetic acid, citric acid, oxalic acid, fumaric acid, maleic acid, succinic acid, malic acid, tartaric acid, aspartic acid, trichloroacetic acid, and trifluoroacetic acid; and salts with sulfonic acids such as methanesulfonic acid, benzenesulfonic acid, p-toluenesulfonic acid, mesitylenesulfonic acid, and naphthalenesulfonic acid.

Specific examples of the compound represented by the formula (1) or a salt thereof include the following compounds.

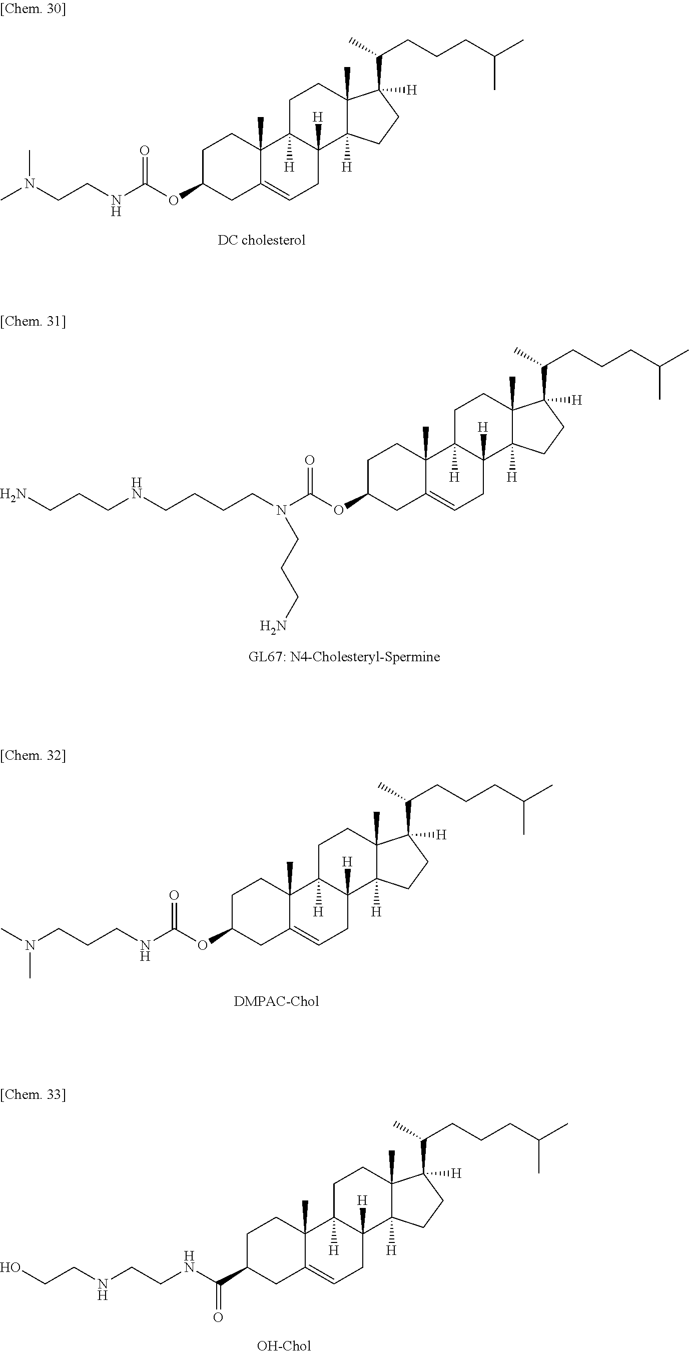

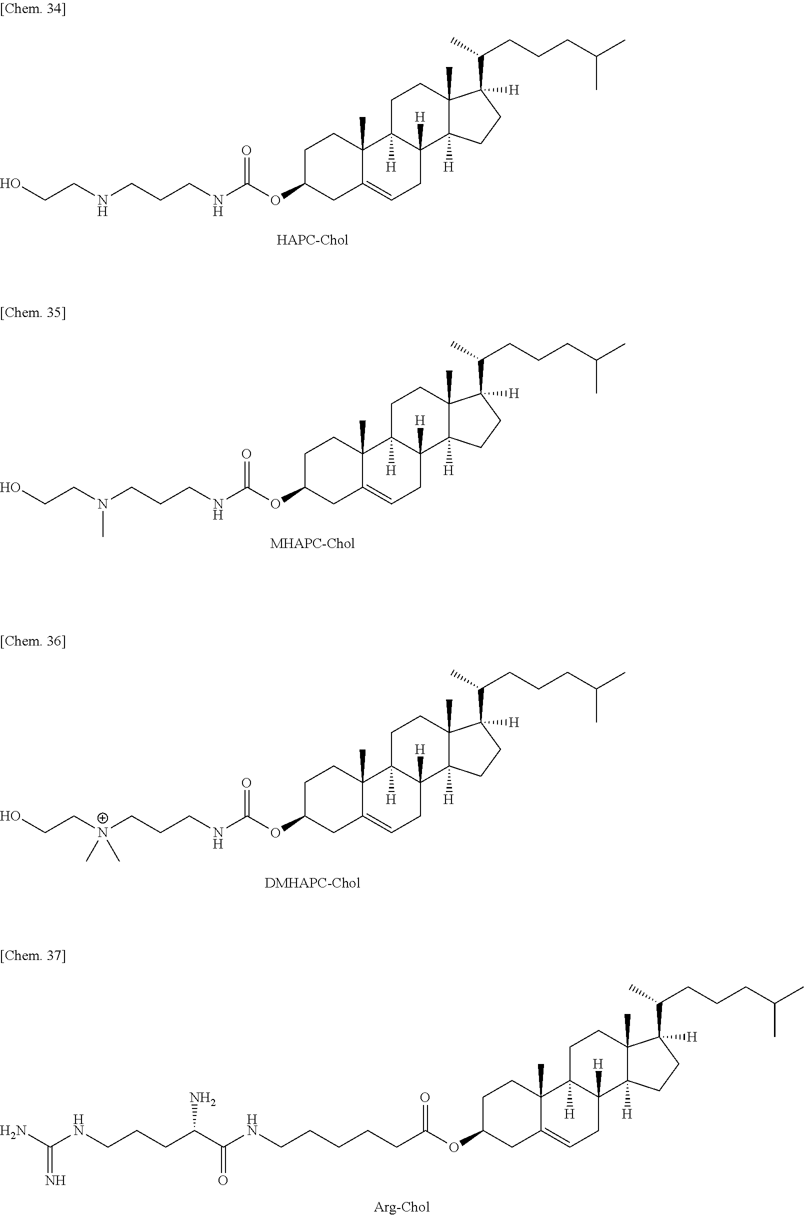

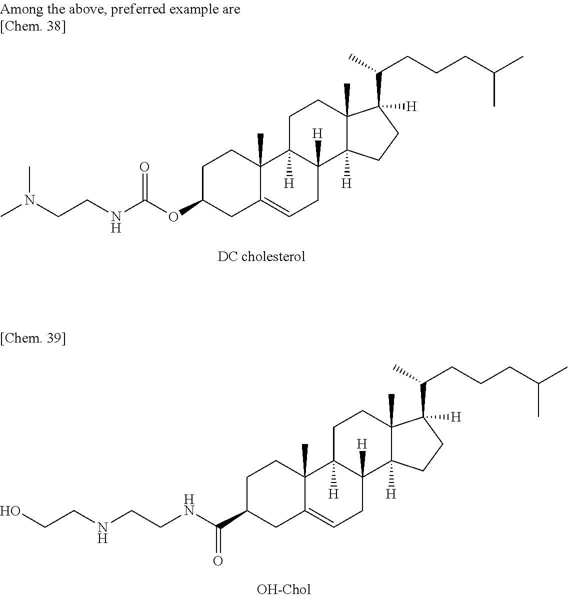

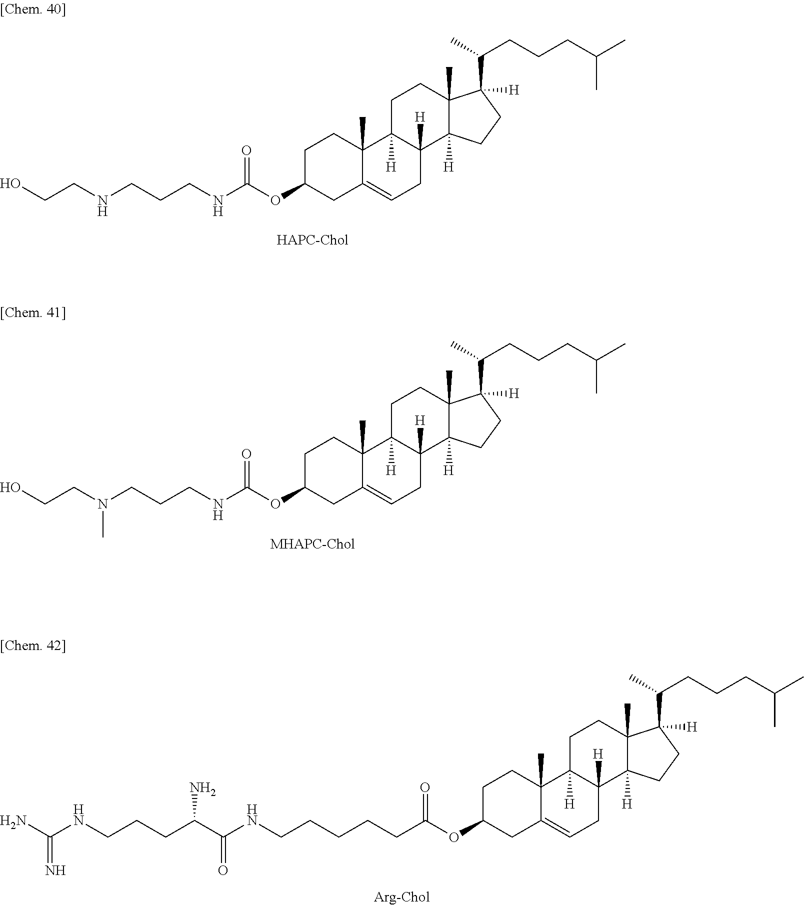

Particularly preferred example are

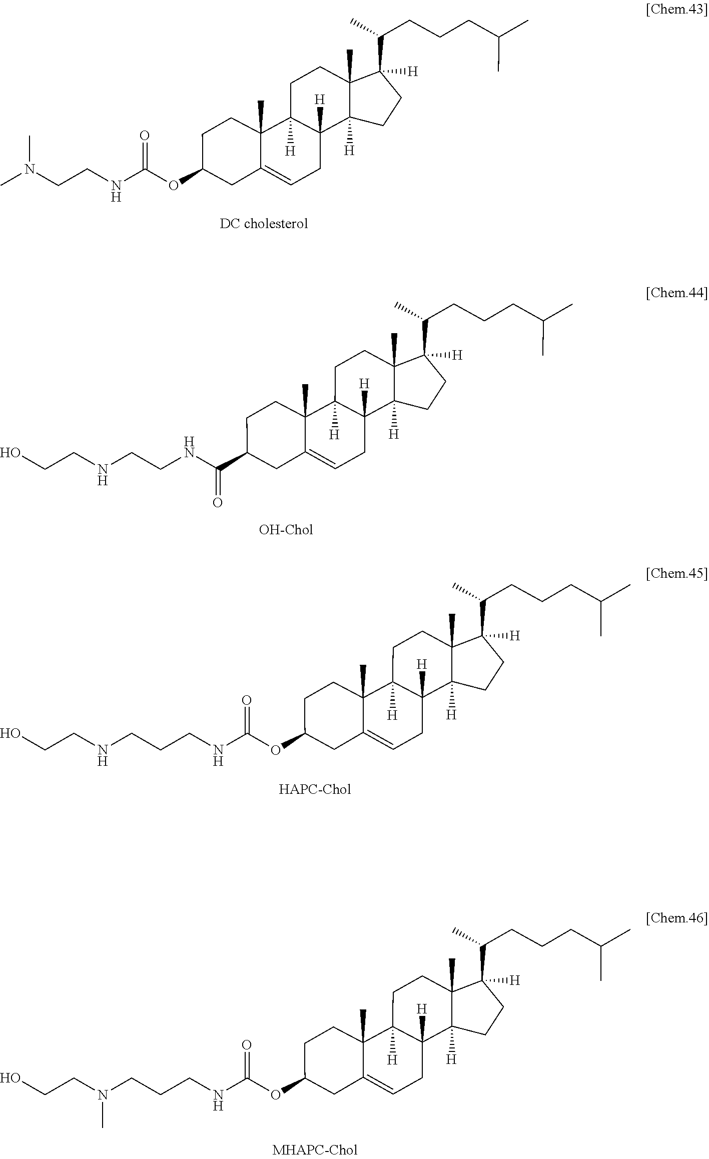

The compound represented by the above formula (1) can be obtained as a commercial product from, for example, Cayman chemicals, and Avanti polar lipids.

The content of the compound represented by the formula (1) is preferably 5 to 80 mol %, preferably 10 to 80 mol %, more preferably 10 mol % to 60 mol %, still more preferably 30 mol % to 50 mol % with respect to the total lipid.

### <Ionizable Lipids>

In the present invention, an ionizable lipid is used. The ionizable lipid may be a lipid having at least one biodegradable group. The ionizable lipid may be a lipid having at least one ionizable amino group and at least one biodegradable group. Examples of the above-mentioned biodegradable group include groups represented by —O(CO)O—, —O(CO)—, or —(CO)O—.

### <<Lipid Represented by Formula (4) or Salt Thereof>>

For example, a lipid represented by Formula (4) or a salt thereof may be used as the ionizable lipid.

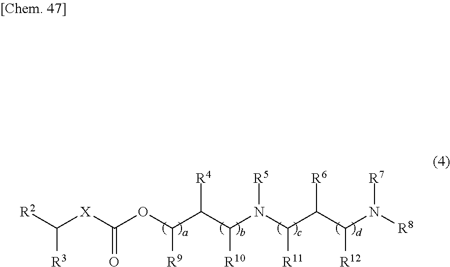

In the formula, X represents —NR¹— or —O—,
R¹ represents a hydrogen atom, a hydrocarbon group having 6 to 24 carbon atoms, or a group represented by R²¹-L¹-R²²—, where R²¹ represents a hydrocarbon group having 1 to 24 carbon atoms, L¹ represents —O(CO)O—, —O(CO)—, —(CO)O—, —O—, or a group represented by the following formula,

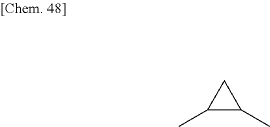

and R²² represents a divalent hydrocarbon linking group having 1 to 18 carbon atoms,
R² and R³ each independently represent a hydrogen atom, a hydrocarbon group having 3 to 24 carbon atoms, or a group represented by R³¹-L²-R³²—, where R³¹ represents a hydrocarbon group having 1 to 24 carbon atoms, L² represents —O(CO)O—, —O(CO)—, —(CO)O—, —O—, or a group represented by the following formula,

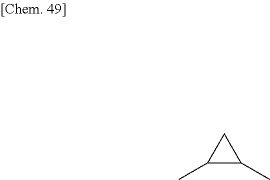

and R³² represents a divalent hydrocarbon linking group having 1 to 18 carbon atoms,
R⁴, R⁵, R⁶, R⁷, R⁸, R⁹, R¹⁰, R¹¹, and R¹² each independently represent a hydrogen atom or an alkyl group having 1 to 18 carbon atoms which may be substituted,
groups in any one or more pairs among R⁴ and R⁵, R¹⁰ and R⁵, R⁵ and R¹², R⁴ and R⁶, R⁵ and R⁶, R⁶ and R⁷, R⁶ and R¹⁰, R¹² and R⁷, and R⁷ and R⁸ may be linked to each other to form a 4- to 7-membered ring which may contain an O atom,
a substituent on the alkyl group having 1 to 18 carbon atoms which may be substituted is a hydroxyl group, a carboxyl group, an amino group represented by —NR⁴⁵R⁴⁶, a substituted or unsubstituted aryl group, a substituted or unsubstituted heteroaryl group, or a group represented by —O(CO)O—R⁴¹, —O(CO)—R⁴², —(CO)O—R⁴³, or —O—R⁴⁴, where R⁴¹, R⁴², R⁴³, R⁴⁴, R⁴⁵, and R⁴⁶ each independently represent a hydrocarbon group having 1 to 18 carbon atoms,
the substituent on the substituted or unsubstituted aryl group and on the substituted or unsubstituted heteroaryl group is an alkyl group having 1 to 18 carbon atoms, a hydroxyl group, a carboxyl group, an amino group represented by —NR⁴⁵R⁴⁶, or a group represented by —O(CO)O—R⁴¹, —O(CO)—R⁴², —(CO)O—R⁴³, or —O—R⁴⁴, where R⁴¹, R⁴², R⁴³, R⁴⁴, R⁴⁵, and R⁴⁶ each independently represent a hydrocarbon group having 1 to 18 carbon atoms, and
a, b, c, and d each independently represent an integer of 0 to 3, a+b is 1 or more, and c+d is 1 or more.

As the hydrocarbon group having 6 to 24 carbon atoms that is represented by R¹ and the hydrocarbon group having 3 to 24 carbon atoms that is represented by R² and R³, an alkyl group, an alkenyl group, or an alkynyl group is preferable, and an alkyl group or an alkenyl group is more preferable. The alkyl group having 6 to 24 carbon atoms and the alkyl group having 3 to 24 carbon atoms may be linear or branched or may be chainlike or cyclic. The alkyl group having 6 to 24 carbon atoms is preferably an alkyl group having 6 to 20 carbon atoms, and the alkyl group having 3 to 24 carbon atoms is more preferably an alkyl group having 6 to 20 carbon atoms. Specifically, examples thereof include a hexyl group, a heptyl group, an octyl group, a nonyl group, a decyl group, an undecyl group, a dodecyl group, a tridecyl group, a trimethyldodecyl group (preferably a 3,7,11-trimethyldodecyl group), a tetradecyl group, a pentadecyl group, a hexadecyl group, a tetramethylhexadecyl group (preferably a 3,7,11,15-tetramethylhexadecyl group), a heptadecyl group, an octadecyl group, a nonadecyl group, an icosyl group, and the like. The alkenyl group having 6 to 24 carbon atoms and the alkenyl group having 3 to 24 carbon atoms may be linear or branched or may be chainlike or cyclic. The alkenyl group having 6 to 24 carbon atoms is preferably an alkenyl group having 6 to 20 carbon atoms, and the alkenyl group having 3 to 24 carbon atoms is more preferably an alkenyl group having 6 to 20 carbon atoms. Specifically, examples thereof include a hexenyl group, a heptenyl group, an octenyl group, a nonenyl group, a decenyl group, an undecenyl group, a dodecenyl group, a dodecadienyl group, a tridecenyl group, a tetradecenyl group, a pentadecenyl group, a hexadecenyl group (preferably a (Z)-hexadec-9-enyl group), a hexadecadienyl group, a heptadecenyl group (preferably a (Z)-heptadec-8-enyl group), a heptadecadienyl group (preferably a (8Z,11Z)-heptadeca-8,11-dienyl group), an octadecenyl group (preferably a (Z)-octadec-9-enyl group), an octadecadienyl group (preferably a (9Z,12Z)-octadeca-9,12-dienyl group), a nonadecenyl group, an icosenyl group (preferably a (Z)-icos-11-enyl group), an icosadienyl group (preferably a (11Z,14Z)-icosa-11,14-dienyl group), and the like. The alkynyl group having 6 to 24 carbon atoms is preferably an alkynyl group having 6 to 20 carbon atoms, and the alkynyl group having 3 to 24 carbon atoms is more preferably an alkynyl group having 6 to 20 carbon atoms. Specifically, examples thereof include a hexynyl group, a heptynyl group, an octynyl group, a nonynyl group, a decynyl group, an undecynyl group, a dodecynyl group, a tetradecynyl group, a pentadecynyl group, a hexadecynyl group, a heptadecynyl group, an octadecynyl group, and the like. All of the above alkenyl groups preferably have one double bond or two double bonds. All of the above alkynyl groups preferably have one triple bond or two triple bonds.

The hydrocarbon group having 1 to 24 carbon atoms that is represented by R²¹ and R³¹ is preferably an alkyl group having 10 to 24 carbon atoms, an alkenyl group having 10 to 24 carbon atoms, or an alkynyl group having 10 to 24 carbon atoms. The alkyl group having 10 to 24 carbon atoms may be linear or branched or may be chainlike or cyclic. The alkyl group having 10 to 24 carbon atoms is preferably an alkyl group having 12 to 24 carbon atoms. Specifically, examples thereof include a decyl group, an undecyl group, a dodecyl group, a tridecyl group, a trimethyldodecyl group (preferably a 3,7,11-trimethyldodecyl group), a tetradecyl group, a pentadecyl group, a hexadecyl group, a tetramethylhexadecyl group (preferably a 3,7,11,15-tetramethylhexadecyl group), a heptadecyl group, an octadecyl group, a 2-butylhexyl group, a 2-butyloctyl group, a 1-pentylhexyl group, a 2-pentylheptyl group, a 3-pentyloctyl group, a 1-hexylheptyl group, a 1-hexylnonyl group, a 2-hexyloctyl group, a 2-hexyldecyl group, a 3-hexylnonyl group, a 1-heptyloctyl group, a 2-heptylnonyl group, a 2-heptylundecyl group, a 3-heptyldecyl group, a 1-octylnonyl group, a 2-octyldecyl group, a 2-octyldodecyl group, a 3-octylundecyl group, a 2-nonylundecyl group, a 3-nonyldodecyl group, a 2-decyldodecyl group, a 2-decyltetradecyl group, a 3-decyltridecyl group, a 2-(4,4-dimethylpentan-2-yl)-5,7,7-trimethyloctyl group, and the like. The alkenyl group having 10 to 24 carbon atoms may be linear or branched or may be chainlike or cyclic. Specifically, examples thereof include a decenyl group, an undecenyl group, a dodecenyl group, a dodecadienyl group, tridecenyl group (preferably a (Z)-tridec-8-enyl group), a tetradecenyl group (preferably a tetradec-9-enyl group), a pentadecenyl group (preferably a (Z)-pentadec-8-enyl group), a hexadecenyl group (preferably a (Z)-hexadec-9-enyl group), a hexadecadienyl group, a heptadecenyl group (preferably a (Z)-heptadec-8-enyl group), a heptadecadienyl group (preferably a (8Z,11Z)-heptadeca-8,11-dienyl group), an octadecenyl group (preferably a (Z)-octadec-9-enyl group), an octadecadienyl group (preferably a (9Z,12Z)-octadeca-9,12-dienyl group), and the like. The alkynyl group having 10 to 24 carbon atoms may be linear or branched or may be chainlike or cyclic. Specifically, examples thereof include a decynyl group, an undecynyl group, a dodecynyl group, a tetradecynyl group, a pentadecynyl group, a hexadecynyl group, a heptadecynyl group, an octadecynyl group, and the like. All of the above alkenyl groups preferably have one double bond or two double bonds. All of the above alkynyl groups preferably have one triple bond or two triple bonds.

The divalent hydrocarbon linking group having 1 to 18 carbon atoms that is represented by R²² and R³² is preferably an alkylene group having 1 to 18 carbon atoms or an alkenylene group having 2 to 18 carbon atoms. The alkylene group having 1 to 18 carbon atoms may be linear or branched or may be chainlike or cyclic. The number of carbon atoms in the alkylene group is preferably 1 to 12, more preferably 1 to 10, and still more preferably 2 to 10. Specifically, examples thereof include a methylene group, an ethylene group, a trimethylene group, a tetramethylene group, a pentamethylene group, a hexamethylene group, a heptamethylene group, an octamethylene group, a nonamethylene group, a decamethylene group, an undecamethylene group, a dodecamethylene group, and the like. The alkenylene group having 2 to 18 carbon atoms may be linear or branched or may be chainlike or cyclic. The number of carbon atoms in the alkenylene group is preferably 1 to 12, and more preferably 2 to 10.
—O(CO)O—, —O(CO)—, and —(CO)O— are in a preferred range of L¹, and —O(CO)— and —(CO)O— are in a more preferred range of L¹.
—O(CO)O—, —O(CO)—, and —(CO)O— are in a preferred range of L², and —O(CO)— and —(CO)O— are in a more preferred range of L².

The alkyl group having 1 to 18 carbon atoms which may be substituted and which represented by R⁴, R⁶, R⁹, R¹⁰, R¹¹, and R¹² may be linear or branched or may be chainlike or cyclic. The number of carbon atoms in the alkyl group is preferably 1 to 12. Specifically, examples thereof include a methyl group, an ethyl group, a propyl group, an isopropyl group, a cyclopropyl group, a butyl group, an isobutyl group, a tert-butyl group, a cyclobutyl group, a pentyl group, a cyclopentyl group, a hexyl group, a cyclohexyl group, a heptyl group, an octyl group, a nonyl group, a decyl group, an undecyl group, a dodecyl group, and the like. In a case where the alkyl group has a substituent, as the substituent, a hydroxyl group, a carboxyl group, or a group represented by —O(CO)O—R⁴¹, —O(CO)—R⁴², —(CO)O—R⁴³, or —O—R⁴⁴ is preferable, and a group represented by —O(CO)—R⁴² or —(CO)O—R⁴³ is more preferable.

The alkyl group having 1 to 18 carbon atoms which may be substituted and which represented by R⁵, R⁷, and R⁸ may be linear or branched or may be chainlike or cyclic. The number of carbon atoms in the alkyl group is preferably 1 to 12, and more preferably 1 to 8. Specifically, examples thereof include a methyl group, an ethyl group, a propyl group, an isopropyl group, a cyclopropyl group, a butyl group, an isobutyl group, a tert-butyl group, a cyclobutyl group, a pentyl group, a cyclopentyl group, a hexyl group, a cyclohexyl group, a heptyl group, an octyl group, a nonyl group, a decyl group, an undecyl group, a dodecyl group, and the like. In a case where the alkyl group has a substituent, as the substituent, a hydroxyl group, a carboxyl group, or a group represented by —O(CO)O—R⁴¹, —O(CO)—R⁴², —(CO)O—R⁴³, or —O—R⁴⁴ is preferable, and a group represented by —O(CO)—R⁴², —(CO)O—R⁴³, or —O—R⁴⁴ is more preferable.

Examples of the 4- to 7-membered ring which may contain an O atom include an azetidine ring, a pyrrolidine ring, a piperidine ring, a morpholine ring, and an azepane ring. The 4- to 7-membered ring is preferably a 6-membered ring and is preferably a piperidine ring or a morpholine ring.

In a case where the alkyl group having 1 to 18 carbon atoms which is represented by R⁴, R⁵, R⁶, R⁷, R¹, R⁹, R¹⁰, R¹¹, and R¹² and which may be substituted has a substituted or unsubstituted aryl group as a substituent, the number of carbon atoms in the aryl group is preferably 6 to 22, more preferably 6 to 18, and still more preferably 6 to 10. Specifically, examples of the aryl group include a phenyl group, a naphthyl group, an anthracenyl group, a phenanthrenyl group, and the like. As the substituent on the aryl group, an alkyl group having 1 to 18 carbon atoms, a hydroxyl group, a carboxyl group, an amino group represented by —NR⁴⁵R⁴⁶, or a group represented by —O(CO)O—R⁴¹, —O(CO)—R⁴², —(CO)O—R⁴³, or —O—R⁴⁴ is preferable, and a hydroxyl group or a carboxyl group is more preferable. Specifically, examples of the substituted aryl group include a hydroxyphenyl group, a carboxyphenyl group, and the like.

In a case where the alkyl group having 1 to 18 carbon atoms which is represented by R⁴, R⁵, R⁶, R⁷, R¹, R⁹, R¹⁰, R¹¹, and R¹² and which may be substituted has a substituted or unsubstituted heteroaryl group as a substituent, the number of carbon atoms in the heteroaryl group is preferably 1 to 12, and more preferably 1 to 6. Specifically, examples of the heteroaryl group include a pyridyl group, a pyrazolyl group, an imidazolyl group, a benzimidazolyl group, a thiazolyl group, an oxazolyl group, and the like. As the substituent on the heteroaryl group, an alkyl group having 1 to 18 carbon atoms, a hydroxyl group, a carboxyl group, an amino group represented by —NR⁴⁵R⁴⁶, or a group represented by —O(CO)O—R⁴¹, —O(CO)—R⁴², —(CO)O—R⁴³, or —O—R⁴⁴ is preferable, and a hydroxyl group or a carboxyl group is more preferable. Specifically, examples of the substituted or unsubstituted heteroaryl group include a hydroxypyridyl group, a carboxypyridyl group, a pyridonyl group, and the like.

As hydrocarbon group having 1 to 18 carbon atoms that is represented by R⁴¹, R⁴², R⁴³, R⁴⁴, R⁴⁵, and R⁴⁶, an alkyl group having 1 to 18 carbon atoms, an alkenyl group having 2 to 18 carbon atoms, or an alkynyl group having 2 to 18 carbon atoms is preferable, and an alkyl group having 1 to 18 carbon atoms or an alkenyl group having 2 to 18 carbon atoms is more preferable. The alkyl group having 1 to 18 carbon atoms may be linear or branched or may be chainlike or cyclic. The number of carbon atoms in the alkyl group is preferably 3 to 18, and more preferably 5 to 18. Specifically, examples thereof include a propyl group, an isopropyl group, a cyclopropyl group, a butyl group, an isobutyl group, a tert-butyl group, a cyclobutyl group, a pentyl group, a cyclopentyl group, a hexyl group, a cyclohexyl group, a heptyl group, an octyl group, a nonyl group, a decyl group, an undecyl group, a dodecyl group, a tridecyl group, a trimethyldodecyl group (preferably a 3,7,11-trimethyldodecyl group), a tetradecyl group, a pentadecyl group, a hexadecyl group, a heptadecyl group, an octadecyl group, and the like. The alkenyl group having 2 to 18 carbon atoms may be linear or branched or may be chainlike or cyclic. The number of carbon atoms in the alkenyl group is preferably 3 to 18, and more preferably 5 to 18. Specifically, examples thereof include an allyl group, a prenyl group, a pentenyl group, a hexenyl group, a heptenyl group, an octenyl group, a nonenyl group (preferably a (Z)-2-nonenyl group or an (E)-2-nonenyl group), a decenyl group, an undecenyl group, a dodecenyl group, a dodecadienyl group, a tridecenyl group (preferably a (Z)-tridec-8-enyl group), a tetradecenyl group (preferably a tetradec-9-enyl group), a pentadecenyl group (preferably a (Z)-pentadec-8-enyl group), a hexadecenyl group (preferably a (Z)-hexadec-9-enyl group), a hexadecadienyl group, a heptadecenyl group (preferably a (Z)-heptadec-8-enyl group), a heptadecadienyl group (preferably a (8Z,11Z)-heptadeca-8,11-dienyl group), an octadecenyl group (preferably a (Z)-octadec-9-enyl group), an octadecadienyl group (preferably a (9Z,12Z)-octadeca-9,12-dienyl group), and the like. The alkynyl group having 2 to 18 carbon atoms may be linear or branched or may be chainlike or cyclic. The number of carbon atoms in the alkynyl group is preferably 3 to 18, and more preferably 5 to 18. Specifically, examples thereof include a propargyl group, a butynyl group, a pentynyl group, a hexynyl group, a heptynyl group, an octynyl group, a nonynyl group, a decynyl group, an undecynyl group, a dodecynyl group, a tetradecynyl group, a pentadecynyl group, a hexadecynyl group, a heptadecynyl group, an octadecynyl group, and the like.

In a case where X represents —NR¹—, R¹ preferably represents a hydrocarbon group having 6 to 24 carbon atoms or a group represented by R²¹-L¹-R²²—. In this case, it is preferable that one of R² and R³ represent a hydrogen atom and the other represent a hydrocarbon group having 6 to 24 carbon atoms or a group represented by R³¹-L²-R³²—.

In a case where X represents —O—, it is preferable that R² and R³ each independently represent a hydrocarbon group having 6 to 24 carbon atoms or a group represented by R³¹-L²-R³²—.

It is preferable that R⁴, R⁶, R⁹, R¹⁰, R¹¹, and R¹² each represent a hydrogen atom.
R⁵ is preferably a hydrogen atom, an alkyl group having 1 to 18 carbon atoms, an alkyl group having 1 to 18 carbon atoms which may be substituted with —O(CO)—R⁴² or —(CO)O—R⁴³, an alkyl group having 1 to 18 carbon atoms which may be substituted with an aryl group, or an alkyl group having 1 to 18 carbon atoms which may be substituted with a hydroxyl group. In a case where R⁵ is an alkyl group, R⁵ may be linked to R⁴, R⁶, R¹⁰, and R¹² to form a ring which may contain an O atom. Particularly, R⁵ is preferably an alkyl group having 1 to 18 carbon atoms, an alkyl group having 1 to 18 carbon atoms which may be substituted with —O(CO)—R⁴² or —(CO)O—R⁴³, an alkyl group having 1 to 12 carbon atoms which may be substituted with an aryl group, or an alkyl group having 1 to 8 carbon atoms which may be substituted with a hydroxyl group, and more preferably an alkyl group having 1 to 18 carbon atoms or an alkyl group having 1 to 18 carbon atoms which may be substituted with —O(CO)— or —(CO)O—R⁴³.
R⁷ and R⁸ preferably each independently represent a hydrogen atom, a hydrocarbon group having 1 to 18 carbon atoms, an alkyl group having 1 to 18 carbon atoms which may be substituted with —O(CO)—R⁴² or —(CO)O—R⁴³, an alkyl group having 1 to 8 carbon atoms which may be substituted with an aryl group, or an alkyl group having 1 to 8 carbon atoms which may be substituted with a hydroxyl group. Alternatively, it is preferable that R⁷ and R⁸ be linked to each other to form a 4- to 7-membered ring which may contain an O atom.
R⁵ is not linked to R⁷ or R⁸ and does not form a ring with R⁷ or R⁸.
a+b is preferably 1 or 2, and more preferably 1. c+d is preferably 1 or 2, and more preferably 1.

The compound represented by Formula (4) is preferably a compound represented by Formula (21).

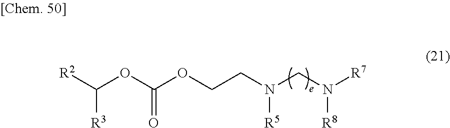

In the formula, R² and R³ each independently represent a hydrocarbon group containing one or more unsaturated bond and having 3 to 24 carbon atoms, or R² and R³ each independently represent a group represented by R³¹-L²-R³²—, or one of R² and R³ represents a group represented by R³¹-L²-R³²— and the other represents a hydrocarbon group having 3 to 24 carbon atoms,
R³¹ represents a hydrocarbon group having 1 to 24 carbon atoms,
L² represents —O(CO)O—, —O(CO)—, —(CO)O—, —O—, or a group represented by the following formula,

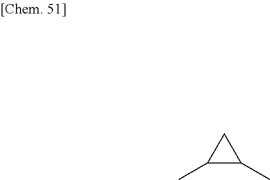

and R³² represents a divalent hydrocarbon linking group having 1 to 18 carbon atoms,
R⁵ represents an alkyl group having 1 to 18 carbon atoms which may be substituted with —O(CO)—R⁴² or —(CO)O—R⁴³ where R⁴² and R⁴³ each independently represent a hydrocarbon group having 1 to 18 carbon atoms,
R⁷ and R⁸ each independently represent an alkyl group having 1 to 4 carbon atoms
e represents 2 or 3.

In formula (21), preferably one of R² and R³ is a group represented by R³¹-L²-R³²—, and the other is a hydrocarbon group having 3 to 24 carbon atoms. In formula (21), L2 preferably represents —O(CO)— or —(CO)O—.

The compound represented by Formula (4) may form a salt.

Examples of the salt in a basic group include salts with mineral acids such as hydrochloric acid, hydrobromic acid, nitric acid, and sulfuric acid; salts with organic carboxylic acids such as formic acid, acetic acid, citric acid, oxalic acid, fumaric acid, maleic acid, succinic acid, malic acid, tartaric acid, aspartic acid, trichloroacetic acid, and trifluoroacetic acid; and salts with sulfonic acids such as methanesulfonic acid, benzenesulfonic acid, p-toluenesulfonic acid, mesitylenesulfonic acid, and naphthalenesulfonic acid.

Examples of the salt in an acidic group include salts with alkali metals such as sodium and potassium; salts with alkaline earth metals such as calcium and magnesium; ammonium salts; salts with nitrogen-containing organic bases such as trimethylamine, triethylamine, tributylamine, pyridine, N,N-dimethylaniline, N-methylpiperidine, N-methylmorpholine, diethylamine, dicyclohexylamine, procaine, dibenzylamine, N-benzyl-β-phenethylamine, 1-ephenamine, and N,N′-dibenzylethylenediamine, and the like.

Among the above salts, for example, pharmacologically acceptable salts are preferable. The lipid represented by the formula (4) and a method for producing the same are described in WO2019/235635A and WO2021/095876A.

### <<Lipid Represented by Formula (1) or Salt Thereof>>

As another example, a lipid represented by Formula (1) or a salt thereof may be used as the ionizable lipid.

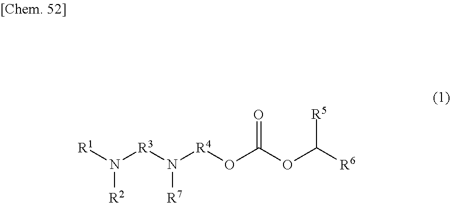

In the formula,
R¹ and R² each independently represent a hydrocarbon group having 1 to 18 carbon atoms, and R³ represents a hydrocarbon group having 2 to 8 carbon atoms, where the hydrocarbon groups represented by R¹, R², and R³ may be substituted with one or more substituents selected from —OH, COOH, —NR⁵¹R⁵², —OC(O)O—R⁵³, —C(O)O—R⁵⁴, —OC(O)—R⁵⁵, and —O—R⁵⁶,
R⁴ represents a hydrocarbon group having 1 to 8 carbon atoms,
R⁵ and R⁶ each independently represent a hydrocarbon group having 1 to 8 carbon atoms or —R⁸-L¹-R⁹, excluding a case that both R⁵ and R⁶ are hydrocarbon groups having 1 to 8 carbon atoms,
R⁷ represents —R¹⁰-L²-R¹¹-L³-R¹²,
R⁵¹ and R⁵² each independently represent a hydrocarbon group having 1 to 8 carbon atoms,
R⁵³, R⁵⁴, R⁵⁵, and R⁵⁶ each independently represent a hydrocarbon group having 1 to 24 carbon atoms,
the hydrocarbon groups represented by R⁵³, R⁵⁴, R⁵⁵, and R⁵⁶ may be substituted with an aryl group having 6 to 20 carbon atoms or —S—R⁵⁸,
the above-described aryl group having 6 to 20 carbon atoms may be substituted with —OH, COOH, —NR⁵¹R⁵², —OC(O)O—R⁵³, —C(O)O—R⁵⁴, —OC(O)—R⁵⁵, —O—R⁵⁶, or -(hydrocarbon group having 1 to 12 carbon atoms)-R⁵⁷,
R⁵⁸ represents a hydrocarbon group having 1 to 12 carbon atoms, and
R⁵⁷ represents —OH, COOH, —NR⁶¹R⁶², —OC(O)O—R⁶³, —C(O)O—R⁶⁴, —OC(O)—R⁶⁵, or —O—R⁶⁶,
R⁶¹ and R⁶² each independently represent a hydrocarbon group having 1 to 8 carbon atoms,
R⁶³, R⁶⁴, R⁶⁵, and R⁶⁶ each independently represent a hydrocarbon group having 1 to 24 carbon atoms,
the hydrocarbon groups represented by R⁶³, R⁶⁴, R⁶⁵, and R⁶⁶ may be substituted with an aryl group having 6 to 20 carbon atoms or —S—R⁶⁸,
the above-described aryl group having 6 to 20 carbon atoms may be substituted with —OH, COOH, —NR⁶¹R⁶², —OC(O)O—R⁶³, —C(O)O—R⁶⁴, —OC(O)—R⁶⁵, —O—R⁶⁶, or -(hydrocarbon group having 1 to 12 carbon atoms)-R⁶⁷,
R⁶⁸ represents a hydrocarbon group having 1 to 12 carbon atoms, and
L¹, L², and L³ each independently represent —OC(O)O—, —C(O)O—, —OC(O)—, or —O—.
R⁸ represents a hydrocarbon group having 1 to 12 carbon atoms,
R⁹ represents a hydrocarbon group having 1 to 24 carbon atoms,
R¹⁰ represents a hydrocarbon group having 1 to 8 carbon atoms,
R¹¹ represents a hydrocarbon group having 1 to 24 carbon atoms,
R¹² represents a hydrocarbon group having 1 to 24 carbon atoms,
the hydrocarbon groups represented by R⁹ and R¹² may be substituted with an aryl group, —OC(O)O—R⁵³, —C(O)O—R⁵⁴, —OC(O)—R⁵⁵, or —S—R⁵⁸, where definitions of R⁵³, R⁵⁴, R⁵⁵, and R⁵⁸ are as described above, and
the hydrocarbon group represented by R¹¹ may be substituted with —OC(O)O—R⁵³, —C(O)O—R⁵⁴, or —OC(O)—R⁵⁵, where the definitions of R⁵³, R⁵⁴, and R⁵⁵ are as described above.

A hydrocarbon group having 1 to 24 carbon atoms, a hydrocarbon group having 1 to 18 carbon atoms, a hydrocarbon group having 1 to 12 carbon atoms, a hydrocarbon group having 2 to 8 carbon atoms, and a hydrocarbon group having 1 to 8 carbon atoms are each preferably an alkyl group, an alkenyl group, or an alkynyl group.

The alkyl group may be linear or branched, or may be chainlike or cyclic. Specifically, examples of the alkyl group include a methyl group, an ethyl group, a propyl group, an isopropyl group, a cyclopropyl group, a butyl group, an isobutyl group, a tert-butyl group, a cyclobutyl group, a pentyl group, a cyclopentyl group, a hexyl group, a cyclohexyl group, a heptyl group, an octyl group, a nonyl group, a decyl group, an undecyl group, a dodecyl group, a tridecyl group, a trimethyldodecyl group (preferably a 3,7,11-trimethyldodecyl group), a tetradecyl group, a pentadecyl group, a hexadecyl group, a tetramethylhexadecyl group (preferably a 3,7,11,15-tetramethylhexadecyl group), a heptadecyl group, an octadecyl group, a 2-butylhexyl group, a 2-butyloctyl group, a 1-pentylhexyl group, a 2-pentylheptyl group, a 3-pentyloctyl group, a 1-hexylheptyl group, a 1-hexylnonyl group, a 2-hexyloctyl group, a 2-hexyldecyl group, a 3-hexylnonyl group, a 1-heptyloctyl group, a 2-heptylnonyl group, a 2-heptylundecyl group, a 3-heptyldecyl group, a 1-octylnonyl group, a 2-octyldecyl group, a 2-octyldodecyl group, a 3-octylundecyl group, a 2-nonylundecyl group, a 3-nonyldodecyl group, a 2-decyldodecyl group, a 2-decyltetradecyl group, a 3-decyltridecyl group, a 2-(4,4-dimethylpentan-2-yl)-5,7,7-trimethyloctyl group, and the like.

The alkenyl group may be linear or branched, or may be chainlike or cyclic. Specifically, examples of the alkenyl group include an allyl group, a prenyl group, a pentenyl group, a hexenyl group, a heptenyl group, an octenyl group, a nonenyl group (preferably a (Z)-2-nonenyl group or an (E)-2-nonenyl group), a decenyl group, an undecenyl group, a dodecenyl group, a dodecadienyl group, a tridecenyl group (preferably a (Z)-tridec-8-enyl group), a tetradecenyl group (preferably a tetradec-9-enyl group), a pentadecenyl group (preferably a (Z)-pentadec-8-enyl group), a hexadecenyl group (preferably a (Z)-hexadec-9-enyl group), a hexadecadienyl group, a heptadecenyl group (preferably a (Z)-heptadec-8-enyl group), a heptadecadienyl group (preferably a (8Z,11Z)-heptadeca-8,11-dienyl group), an octadecenyl group (preferably a (Z)-octadec-9-enyl group), an octadecadienyl group (preferably a (9Z,12Z)-octadeca-9,12-dienyl group), and the like.

The alkynyl group may be linear or branched, or may be chainlike or cyclic. Specifically, examples of alkynyl group include a propargyl group, a butynyl group, a pentynyl group, a hexynyl group, a heptynyl group, an octynyl group, a nonynyl group, a decynyl group, an undecynyl group, a dodecynyl group, a tetradecynyl group, a pentadecynyl group, a hexadecynyl group, a heptadecynyl group, an octadecynyl group, and the like.

All of the above alkenyl groups preferably have one double bond or two double bonds. All of the above alkynyl groups preferably have one triple bond or two triple bonds.

The hydrocarbon group having 1 to 12 carbon atoms in -(hydrocarbon group having 1 to 12 carbon atoms)-R⁶⁷ is preferably an alkylene group having 1 to 12 carbon atoms or an alkenylene group having 2 to 12 carbon atoms. The alkylene group having 1 to 12 carbon atoms and the alkenylene group having 2 to 12 carbon atoms may be linear or branched, or may be chainlike or cyclic.

Specifically, examples thereof include a methylene group, an ethylene group, a trimethylene group, a tetramethylene group, a pentamethylene group, a hexamethylene group, a heptamethylene group, an octamethylene group, a nonamethylene group, a decamethylene group, an undecamethylene group, and the like.

The aryl group preferably has 6 to 20 carbon atoms, more preferably has 6 to 18 carbon atoms, and even more preferably 6 to 10 carbon atoms. Specifically, examples of the aryl group include a phenyl group, a naphthyl group, an anthracenyl group, a phenanthrenyl group, and the like.

R¹ and R² each independently preferably represent a hydrocarbon group having 1 to 12 carbon atoms, more preferably represent a hydrocarbon group having 1 to 6 carbon atoms, and even more preferably represent a hydrocarbon group having 1 to 3 carbon atoms.
R³ preferably represents a hydrocarbon group having 2 to 6 carbon atoms and more preferably represents a hydrocarbon group having 2 to 4 carbon atoms.

The hydrocarbon groups represented by R¹, R², and R³ may be preferably substituted with —OH.
L¹ and L³ each independently preferably represent —C(O)O— or —OC(O)—.
L² preferably represents —OC(O)O—, —C(O)O—, or —OC(O)—.

R⁸ preferably represents a hydrocarbon group having 1 to 10 carbon atoms and more preferably represents a hydrocarbon group having 1 to 8 carbon atoms.
R⁹ preferably represents a hydrocarbon group having 1 to 20 carbon atoms and more preferably represents a hydrocarbon group having 1 to 16 carbon atoms.
R¹¹ preferably represents a hydrocarbon group having 1 to 16 carbon atoms and more preferably represents a hydrocarbon group having 1 to 9 carbon atoms.
R¹² preferably represents a hydrocarbon group having 1 to 20 carbon atoms and more preferably represents a hydrocarbon group having 1 to 16 carbon atoms.

The hydrocarbon groups represented by R⁹ and R¹² may be preferably substituted with an aryl group or —S—R⁵⁸. Here, R⁵⁸ preferably represents a hydrocarbon group having 1 to 8 carbon atoms.

The hydrocarbon group represented by R¹¹ may be preferably substituted with —C(O)O—R⁵⁵ or —OC(O)—R⁵⁶, where R⁵⁵ and R⁵⁶ each independently represent a hydrocarbon group having 1 to 16 carbon atoms.

The hydrocarbon groups represented by R⁵⁵ and R⁵⁶ may be preferably substituted with an aryl group having 6 to 20 carbon atoms or —S—R⁵⁸, and the definition of R⁵⁸ is as described above.

The compound represented by Formula (1) is preferably a compound represented by Formula (1-1) as a first example.

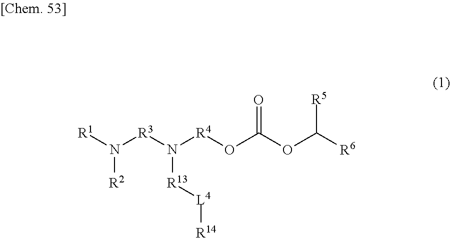

In the formula,
R¹ and R² each independently represent a hydrocarbon group having 1 to 18 carbon atoms, and R³ represents a hydrocarbon group having 2 to 8 carbon atoms, where the hydrocarbon groups represented by R¹, R², and R³ may be substituted with —OH, COOH, —NR⁵¹R⁵², —OC(O)O—R⁵³, —C(O)O—R⁵⁴, —OC(O)—R⁵⁵, or —O—R⁵⁶,
R⁴ represents a hydrocarbon group having 1 to 8 carbon atoms,
R⁵ and R⁶ each independently represent a hydrocarbon group having 1 to 8 carbon atoms or —R⁸-L¹-R⁹, excluding a case that both R⁵ and R⁶ are hydrocarbon groups having 1 to 8 carbon atoms,
L¹ represents —OC(O)O—, —C(O)O—, —OC(O)—, or —O—,
R⁸ represents a hydrocarbon group having 1 to 12 carbon atoms,
R⁹ represents a hydrocarbon group having 1 to 24 carbon atoms, where the hydrocarbon group represented by R⁹ may be substituted with an aryl group, —OC(O)O—R⁵³, —C(O)O—R⁵⁴, —OC(O)—R⁵, or —S—R⁵⁸,
R⁵¹ and R⁵² each independently represent a hydrocarbon group having 1 to 8 carbon atoms,
R⁵³, R⁵⁴, R⁵⁵, and R⁵⁶ each independently represent a hydrocarbon group having 1 to 24 carbon atoms,
the hydrocarbon groups represented by R⁵³, R⁵⁴, R⁵⁵, and R⁵⁶ may be substituted with an aryl group having 6 to 20 carbon atoms or —S—R⁵⁸,
the above-described aryl group having 6 to 20 carbon atoms may be substituted with —OH, COOH, —NR⁵¹R⁵², —OC(O)O—R⁵³, —C(O)O—R⁵⁴, —OC(O)—R⁵⁵, —O—R⁵⁶, or -(hydrocarbon group having 1 to 12 carbon atoms)-R⁵⁷,
R⁵⁸ represents a hydrocarbon group having 1 to 12 carbon atoms, and
R⁵⁷ represents —OH, COOH, —NR⁵¹R⁵², —OC(O)O—R⁵³, —C(O)O—R⁵⁴, —OC(O)—R⁵⁵, or —O—R⁵⁶.
R¹³ represents a hydrocarbon group having 1 to 8 carbon atoms,
R¹⁴ represents —R¹⁵-L⁵-R¹⁶, where R¹⁵ represents a hydrocarbon group having 1 to 24 carbon atoms, L⁵ represents —OC(O)O—, —C(O)O—, —OC(O)—, or —O—, and R¹⁶ represents a hydrocarbon group having 1 to 24 carbon atoms,
the hydrocarbon group having 1 to 24 carbon atoms represented by R¹⁵ may be substituted with —OC(O)O—R⁵³, —C(O)O—R⁵⁴, or —OC(O)—R⁵⁵, where definitions of R⁵³, R⁵⁴, and R⁵⁵ are as described above, and
the hydrocarbon group having 1 to 24 carbon atoms represented by R¹⁶ may be substituted with an aryl group having 6 to 20 carbon atoms, —OC(O)O—R⁵³, —C(O)O—R⁵⁴, —OC(O)—R⁵⁵ or —S—R⁵⁸, where the definitions of R⁵³, R⁵⁴, R⁵⁵, and R⁵⁸ are as described above.

In Formula (1-1), R¹ and R² each independently preferably represent a hydrocarbon group having 1 to 12 carbon atoms, more preferably represent a hydrocarbon group having 1 to 6 carbon atoms, and even more preferably represent a hydrocarbon group having 1 to 3 carbon atoms.

R³ preferably represents a hydrocarbon group having 2 to 6 carbon atoms and more preferably represents a hydrocarbon group having 2 to 4 carbon atoms.

The hydrocarbon groups represented by R¹, R², and R³ may be preferably substituted with —OH.
L¹ preferably represents —C(O)O— or —OC(O)—.
R⁸ preferably represents a hydrocarbon group having 1 to 10 carbon atoms and more preferably represents a hydrocarbon group having 1 to 8 carbon atoms.
R⁹ preferably represents a hydrocarbon group having 1 to 18 carbon atoms, and the hydrocarbon group represented by R⁹ may be substituted with an aryl group having 6 to 20 carbon atoms or —S—R⁵⁸.
R¹⁴ preferably represents —R¹⁵-L⁵-R¹⁶, where R¹⁵ represents a hydrocarbon group having 1 to 18 carbon atoms, L⁵ represents —OC(O)O—, and R¹⁶ represents a hydrocarbon group having 1 to 18 carbon atoms.

The hydrocarbon group having 1 to 18 carbon atoms represented by R¹⁵ may be preferably substituted with —C(O)O—R⁵⁵ or —OC(O)—R⁵⁶. R⁵⁵ and R⁵⁶ each independently represent a hydrocarbon group having 1 to 16 carbon atoms, and the hydrocarbon groups represented by R⁵⁵ and R⁵⁶ may be substituted with an aryl group having 6 to 20 carbon atoms or —S—R⁵⁸ where the definition of R⁵⁸ is as described above.

The hydrocarbon group having 1 to 18 carbon atoms represented by R¹⁶ may be preferably substituted with an aryl group or —S—R⁵⁸, where the definition of R⁵⁸ is as described above.

The compound represented by Formula (1) is preferably a compound represented by Formula (1-2) as a second example.

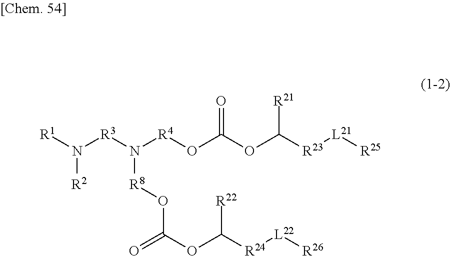

In the formula,
R¹ and R² each independently represent a hydrocarbon group having 1 to 18 carbon atoms, and R³ represents a hydrocarbon group having 2 to 8 carbon atoms, where the hydrocarbon groups represented by R¹, R², and R³ may be substituted with —OH, COOH, —NR⁵¹R⁵², —OC(O)O—R⁵³, —C(O)O—R⁵⁴, —OC(O)—R⁵⁵, or —O—R⁵⁶,
R⁴ and R⁸ each independently represent a hydrocarbon having 1 to 8 carbon atoms,
R²¹ and R²² each independently represent a hydrocarbon group having 1 to 18 carbon atoms,
R²³ and R²⁴ each independently represent a hydrocarbon group having 1 to 12 carbon atoms,
R²⁵ and R²⁶ each independently represent a hydrocarbon group having 1 to 24 carbon atoms,
L²¹ and L²² each independently represent —OC(O)O—, —C(O)O—, —OC(O)—, or —O—,
the hydrocarbon groups represented by R²⁵ and R²⁶ may be substituted with an aryl group having 6 to 20 carbon atoms, —OC(O)O—R⁵³, —C(O)O—R⁵⁴, —OC(O)—R⁵⁵, or —S—R⁵⁸
R⁵¹ and R⁵² each independently represent a hydrocarbon group having 1 to 8 carbon atoms,
R⁵³, R⁵⁴, R⁵⁵, and R⁵⁶ each independently represent a hydrocarbon group having 1 to 18 carbon atoms,
the above-described aryl group having 6 to 20 carbon atoms may be substituted with OH, COOH, —NR⁵¹R⁵², —OC(O)O—R⁵³, —C(O)O—R⁵⁴, —OC(O)—R⁵⁵, —O—R⁵⁶, or -(hydrocarbon group having 1 to 12 carbon atoms)-R⁵⁷, and
R⁵⁷ represents —OH, COOH, —NR⁵¹R⁵², —OC(O)O—R⁵³, —C(O)O—R⁵⁴, —OC(O)—R⁵⁵, or —O—R⁵⁶
R⁵⁸ represents a hydrocarbon group having 1 to 12 carbon atoms.

In Formula (1-2), R¹ and R² each independently preferably represent a hydrocarbon group having 1 to 12 carbon atoms, more preferably represent a hydrocarbon group having 1 to 6 carbon atoms, and even more preferably represent a hydrocarbon group having 1 to 3 carbon atoms. The hydrocarbon groups represented by R¹ and R² may be preferably substituted with —OH, but has more preferably a hydrocarbon having no substituent.
R³ preferably represents a hydrocarbon group having 2 to 6 carbon atoms and more preferably represents a hydrocarbon group having 2 to 4 carbon atoms.
R²¹ and R²² each independently preferably represent a hydrocarbon group having 1 to 12 carbon atoms, more preferably represent a hydrocarbon group having 1 to 8 carbon atoms, and even more preferably represent a hydrocarbon group having 1 to 6 carbon atoms.
R²³ and R²⁴ each independently preferably represent a hydrocarbon group having 1 to 10 carbon atoms and more preferably represent a hydrocarbon group having 1 to 8 carbon atoms.
R²⁵ and R²⁶ each independently preferably represent a hydrocarbon group having 1 to 20 carbon atoms, more preferably represent a hydrocarbon group having 1 to 16 carbon atoms, and even more preferably represent a hydrocarbon group having 1 to 12 carbon atoms.
L²¹ and L²² each independently preferably represent —C(O)O— or —OC(O)—.

The compound represented by Formula (1) is preferably a compound represented by Formula (1-3) as a third example.

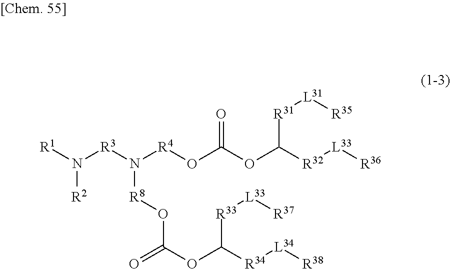

In the formula,
R¹ and R² each independently represent a hydrocarbon group having 1 to 18 carbon atoms, and R³ represents a hydrocarbon group having 2 to 8 carbon atoms, where the hydrocarbon groups represented by R¹, R², and R³ may be substituted with —OH, COOH, —NR⁵¹R⁵², —OC(O)O—R⁵³, —C(O)O—R⁵⁴, —OC(O)—R⁵⁵, or —O—R⁵⁶,
R⁴ and R⁸ each independently represent a hydrocarbon group having 1 to 8 carbon atoms,
R³¹, R³², R³³, and R³⁴ each independently represent a hydrocarbon group having 1 to 12 carbon atoms,
R³⁵, R³⁶, R³⁷, and R³⁸ each independently represent a hydrocarbon group having 1 to 24 carbon atoms,
L³¹, L³², L³³, and L³⁴ each independently represent —OC(O)O—, —C(O)O—, —OC(O)—, or —O—, the hydrocarbon groups represented by R³⁵, R³⁶, R³⁷, and R³⁸ may be substituted with an aryl group having 6 to 20 carbon atoms, —OC(O)O—R⁵³, —C(O)O—R⁵⁴, —OC(O)—R⁵⁵, or S—R⁵⁸,
R⁵¹ and R⁵² each independently represent a hydrocarbon group having 1 to 8 carbon atoms,
R⁵³, R⁵⁴, R⁵⁵, and R⁵⁶ each independently represent a hydrocarbon group having 1 to 18 carbon atoms,
the above-described aryl group having 6 to 20 carbon atoms may be substituted with OH, COOH, —NR⁵¹R⁵², —OC(O)O—R⁵³, —C(O)O—R⁵⁴, —OC(O)—R⁵⁵, —O—R⁵⁶, or -(hydrocarbon group having 1 to 12 carbon atoms)-R⁵⁷, and
R⁵⁷ represents —OH, COOH, —NR⁵¹R⁵², —OC(O)O—R⁵³, —C(O)O—R⁵⁴, —OC(O)—R⁵⁵, or —O—R⁵⁶
R⁵⁸ represents a hydrocarbon group having 1 to 12 carbon atoms.

In Formula (1-3), R¹ and R² each independently preferably represent a hydrocarbon group having 1 to 12 carbon atoms, more preferably represent a hydrocarbon group having 1 to 6 carbon atoms, and even more preferably represent a hydrocarbon group having 1 to 3 carbon atoms. The hydrocarbon groups represented by R¹ and R² may be preferably substituted with —OH, but has more preferably a hydrocarbon having no substituent.
R³ preferably represents a hydrocarbon group having 2 to 6 carbon atoms and more preferably represents a hydrocarbon group having 2 to 4 carbon atoms.
R³¹, R³², R³³, and R³⁴ each independently preferably represent a hydrocarbon group having 1 to 10 carbon atoms, more preferably represent a hydrocarbon group having 1 to 8 carbon atoms, and even more preferably represent a hydrocarbon group having 1 to 3 carbon atoms.
R³⁵, R³⁶, R³⁷, and R³⁸ each independently preferably represent a hydrocarbon group having 1 to 20 carbon atoms, more preferably represent a hydrocarbon group having 1 to 16 carbon atoms, and even more preferably represent a hydrocarbon group having 1 to 12 carbon atoms. The hydrocarbon groups represented by R³⁵, R³⁶, R³⁷, and R³⁸ may be preferably substituted with an aryl group having 6 to 20 carbon atoms or S—R⁵⁸. More preferably, these may be substituted with —S—R⁵⁸.
R³⁵, R³⁶, R³⁷, and R³⁸ each independently particularly preferably represent a hydrocarbon group having 1 to 12 carbon atoms substituted with —S—R⁵⁸, or a hydrocarbon group having 1 to 12 carbon atoms.
L³¹, L³², L³³, and L³⁴ each independently preferably represent —C(O)O—, or —OC(O)—.
R⁵⁸ preferably represents a hydrocarbon group having 1 to 10 carbon atoms and more preferably represents a hydrocarbon group having 1 to 8 carbon atoms.

The compound according to the embodiment of the present invention may form a salt.

Examples of the salt in a basic group include salts with mineral acids such as hydrochloric acid, hydrobromic acid, nitric acid, and sulfuric acid; salts with organic carboxylic acids such as formic acid, acetic acid, citric acid, oxalic acid, fumaric acid, maleic acid, succinic acid, malic acid, tartaric acid, aspartic acid, trichloroacetic acid, and trifluoroacetic acid; and salts with sulfonic acids such as methanesulfonic acid, benzenesulfonic acid, p-toluenesulfonic acid, mesitylenesulfonic acid, and naphthalenesulfonic acid.

Examples of the salt in an acidic group include salts with alkali metals such as sodium and potassium; salts with alkaline earth metals such as calcium and magnesium; ammonium salts; salts with nitrogen-containing organic bases such as trimethylamine, triethylamine, tributylamine, pyridine, N,N-dimethylaniline, N-methylpiperidine, N-methylmorpholine, diethylamine, dicyclohexylamine, procaine, dibenzylamine, N-benzyl-β-phenethylamine, 1-ephenamine, and N,N′-dibenzylethylenediamine; and the like.

Among the above-described salts, for example, pharmacologically acceptable salts are preferable.

The lipid represented by the formula (1) and a method for producing the same are described in WO2022/230964A, the entire of which is incorporated herein by reference.

### <<Lipid Represented by the Formula (5) or a Salt Thereof>>

For example, a lipid represented by Formula (5) or a salt thereof may be used as the ionizable lipid.

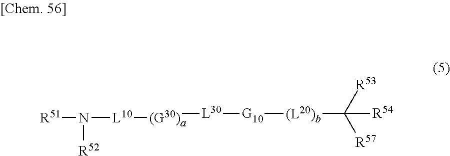

wherein R⁵¹ and R⁵² each independently represent a hydrocarbon group having 1 to 21 carbon atoms which may have a substituent A,
the substituent A represents a hydroxyl group, or a group represented by -G²⁰-CH(R⁵⁵)(R⁵⁶), —N(R⁵⁸)(R⁵⁹) or -G²⁰-R⁶⁰,
G²⁰ represents —O(CO)—, or —(CO)O—,
R⁵⁵ and R⁵⁶ each independently represent a hydrogen atom or a hydrocarbon group having 1 to 18 carbon atoms,
R⁵⁸ and R⁵⁹ each independently represent a hydrogen atom or a cyclic hydrocarbon group having 3 to 6 carbon atoms which may have a substituent B,
the substituent B is —N(R⁶¹)(R⁶²),
R⁶¹ and R⁶² each independently represent a hydrogen atom or a hydrocarbon group having 1 to 6 carbon atoms,
R⁶⁰ represents a hydrocarbon group having 1 to 18 carbon atoms,
L¹⁰ represents a hydrocarbon group having 1 to 18 carbon atoms,
G³⁰ indicates —S—(CO)—NR⁶⁴,
R⁶⁴ represents a group represented by -L³⁰-G²⁰-CH(R⁵⁵)(R⁵⁶) a represents 0 or 1,
L³⁰ represents a single bond or a hydrocarbon group having 1 to 18 carbon atoms,
G¹⁰ represents —O(CO)—, —(CO)O—, —O(CO)O—, or —N(C(O)R⁶³)—,
R⁶³ represents a hydrocarbon group having 1 to 18 carbon atoms,
L²⁰ represents a hydrocarbon group having 1 to 6 carbon atoms,
b represents 0 or 1,
R⁵³, R⁵⁴ and R⁵⁷ each independently represent a hydrogen atom or a hydrocarbon group having 1 to 21 carbon atoms which may have a substituent C,
the substituent C represents a group represented by —(CO)O R⁶⁵ or —O(CO)—R⁶⁵,
R⁶⁵ represents a hydrocarbon group having 1 to 18 carbon atoms or a group represented by -L⁴⁰-CH(R⁶⁶)(R⁶⁷),
L⁴⁰ represents a hydrocarbon group having 1 to 6 carbon atoms,
R⁶⁶ and R⁶⁷ represent a hydrocarbon group having 1 to 10 carbon atoms or an alkoxy group.

The compound represented by Formula (5) may be a compound represented by Formula (5A):

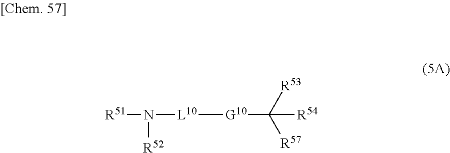

wherein R⁵¹ and R⁵² each independently represent a hydrocarbon group having 1 to 21 carbon atoms which may have a substituent A,
the substituent A represents a hydroxyl group, or a group represented by -G²⁰-CH(R⁵⁵)(R⁵⁶),
G²⁰ represents —O(CO)—, or —(CO)O—,
R⁵⁵ and R⁵⁶ each independently represent a hydrocarbon group having 1 to 18 carbon atoms,
L¹⁰ represents a hydrocarbon group having 1 to 18 carbon atoms,
G¹⁰ represents —O(CO)—, or —(CO)O—,
R⁶³ represents a hydrocarbon group having 1 to 18 carbon atoms,
R⁵³, R⁵⁴ and R⁵⁷ each independently represent a hydrogen atom or a hydrocarbon group having 1 to 21 carbon atoms.

The compound represented by Formula (5) may be a compound represented by Formula (5B):

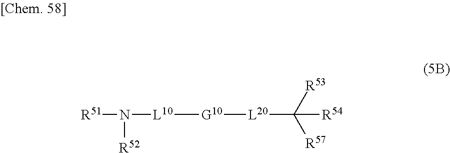

wherein R⁵¹ and R⁵² each independently represent a hydrocarbon group having 1 to 21 carbon atoms,
L¹⁰ represents a hydrocarbon group having 1 to 18 carbon atoms,
G¹⁰ represents —O(CO)O—,
L²⁰ represents a hydrocarbon group having 1 to 6 carbon atoms,
R⁵³, R⁵⁴ and R⁵⁷ each independently represent a hydrogen atom or a hydrocarbon group having 1 to 21 carbon atoms which may have a substituent C,
the substituent C represents a group represented by —O(CO)—R⁶⁵,
R⁶⁵ represents a hydrocarbon group having 1 to 18 carbon atoms or a group represented by -L⁴⁰-CH(R⁶⁶)(R⁶⁷),
L⁴⁰ represents a hydrocarbon group having 1 to 6 carbon atoms,
R⁶⁶ and R⁶⁷ represent an alkoxy group having 1 to 10 carbon atoms.

The compound represented by Formula (5) may be a compound represented by Formula (5C):

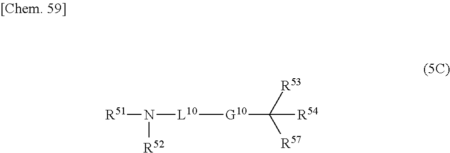

wherein R⁵¹ and R⁵² each independently represent a hydrocarbon group having 1 to 21 carbon atoms
L¹⁰ represents a hydrocarbon group having 1 to 18 carbon atoms,
G¹⁰ represents —N(C(O)R⁶³)—,
R⁶³ represents a hydrocarbon group having 1 to 18 carbon atoms,
R⁵³, R⁵⁴ and R⁵⁷ each independently represent a hydrogen atom or a hydrocarbon group having 1 to 21 carbon atoms which may have a substituent C,
the substituent C represents a group represented by —(CO)O R⁶⁵,
R⁶⁵ represents a group represented by -L⁴⁰-CH(R⁶⁶)(R⁶⁷)
L⁴⁰ represents a hydrocarbon group having 1 to 6 carbon atoms,
R⁶⁶ and R⁶⁷ represent a hydrocarbon group having 1 to 10 carbon atoms.

The compound represented by Formula (5) may be a compound represented by Formula (5D):

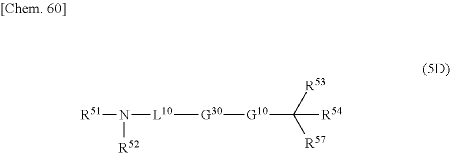

wherein R⁵¹ and R⁵² each independently represent a hydrocarbon group having 1 to 21 carbon atoms,
L¹⁰ represents a hydrocarbon group having 1 to 18 carbon atoms,
G³⁰ indicates —S—(CO)—NR⁶⁴,
R⁶⁴ represents a group represented by -L³⁰-G²⁰-CH(R⁵⁵)(R⁵⁶),
L³⁰ represents a single bond or a hydrocarbon group having 1 to 18 carbon atoms,
G²⁰ represents —(CO)O—,
R⁵⁵ and R⁵⁶ each independently represent a hydrogen atom or a hydrocarbon group having 1 to 18 carbon atoms,
G¹⁰ represents —(CO)O—,
R⁵³, R⁵⁴ and R⁵⁷ each independently represent a hydrogen atom or a hydrocarbon group having 1 to 21 carbon atoms.

The hydrocarbon group having 1 to 21 carbon atoms is preferably an alkyl group having 1 to 21 carbon atoms, an alkenyl group having 2 to 21 carbon atoms, or an alkynyl group having 2 to 21 carbon atoms, more preferably an alkyl group having 1 to 21 carbon atoms, or an alkenyl group having 2 to 21 carbon atoms. The alkyl group having 1 to 21 carbon atoms may be linear or branched, and may be chain or cyclic. The number of carbon atoms is preferably 3 to 21, and more preferably 5 to 21 carbon atoms. Examples include propyl group, isopropyl group, cyclopropyl group, butyl group, isobutyl group, tert-butyl group, cyclobutyl group, pentyl group, cyclopentyl group, hexyl group, cyclohexyl group, heptyl group, octyl group, nonyl group, decyl group, undecyl group, dodecyl group, tridecyl group, trimethyldodecyl group (preferably a 3,7,11-trimethyldodecyl group), tetradecyl group, pentadecyl group, hexadecyl group, heptadecyl group and octadecyl group. The alkenyl group having 2 to 18 carbon atoms may be linear or branched, and may be chain or cyclic. The number of carbon atoms is preferably 3 to 18, and more preferably 5 to 18. Examples include allyl group, prenyl group, pentanyl group, hexenyl group, heptenyl group, octenyl group, nonenyl group (preferably (Z)-2-nonenyl group or (E)-2-nonenyl group), decenyl group, undecenyl group, dodecenyl group, dodecadienyl group, tridecenyl group (preferably (Z)-trideca-8-enyl group), tetradecenyl group (preferably tetradeca-9-enyl group), pentadecenyl group (preferably (Z)-pentadeca-8-enyl group), hexadecenyl group (preferably (Z)-hexadeca-9-enyl group), hexadecadienyl group, heptadecenyl group (preferably (Z)-heptadeca-8-enyl group), heptadecadienyl group (preferably (8Z, 11Z)-heptadeca-8,11-dienyl group), octadecenyl group (preferably (Z)-octadeca-9-enyl group), octadecadienyl Groups (preferably (9Z, 12Z)-octadeca-9,12-dienyl group). The alkynyl group having 2 to 21 carbon atoms may be linear or branched, and may be chain or cyclic. The number of carbon atoms is preferably 3 to 21, and more preferably 5 to 21 carbon atoms. Examples include propargyl group, butynyl group, pentynyl group, hexynyl group, heptynyl group, octynyl group, nonynyl group, decynyl group, undecynyl group, dodecynyl group, tetradecynyl group, pentadecynyl group, hexadecynyl group, heptadecynyl group, octadecynyl group and the like. Examples of the hydrocarbon group having 1 to 18 carbon atoms include those having 1 to 18 carbon atoms among the hydrocarbon groups having 1 to 21 carbon atoms.

As the cyclic hydrocarbon group, a cycloalkyl group having 3 to 10 carbon atoms, a cycloalkenyl group having 3 to 10 carbon atoms, a cycloalkynyl group having 3 to 10 carbon atoms, and an aryl group having 6 to 10 carbon atoms are preferable.

The hydrocarbon group having 1 to 6 carbon atoms is preferably an alkyl group having 1 to 6 carbon atoms, an alkenyl group having 2 to 6 carbon atoms or an alkynyl group having 2 to 6 carbon atoms, and more preferably an alkyl group having 1 to 6 carbon atoms or an alkenyl group having 2 to 6 carbon atoms. The alkyl group having 1 to 6 carbon atoms may be linear or branched, and may be chain or cyclic. Specific examples thereof include propyl group, isopropyl group, cyclopropyl group, butyl group, isobutyl group, tert-butyl group, cyclobutyl group, pentyl group, cyclopentyl group and hexyl group. The alkenyl group having 2 to 6 carbon atoms may be linear or branched, and may be chain or cyclic. Specific examples thereof include allyl group, prenyl group, pentenyl group, and hexenyl group. The alkynyl group having 2 to 6 carbon atoms may be linear or branched, and may be chain or cyclic. Specific examples thereof include propargyl group, butynyl group, pentynyl group, and hexynyl group.

The hydrocarbon group having 1 to 10 carbon atoms is preferably an alkyl group having 1 to 10 carbon atoms, an alkenyl group having 2 to 10 carbon atoms, or an alkynyl group having 2 to 10 carbon atoms, and preferably an alkyl group having 1 to 10 carbon atoms or an alkenyl group having 2 to 10 carbon atoms. The alkyl group having 1 to 10 carbon atoms may be linear or branched, and may be chain or cyclic. The number of carbon atoms is preferably 3 to 10, and more preferably 5 to 10 carbon atoms. Specific example include propyl group, isopropyl group, cyclopropyl group, butyl group, isobutyl group, tert-butyl group, cyclobutyl group, pentyl group, cyclopentyl group, hexyl group, cyclohexyl group, heptyl group, octyl group, nonyl group, and decyl group. The alkenyl group having 2 to 10 carbon atoms may be linear or branched, and may be chain or cyclic. The number of carbon atoms is preferably 3 to 10, more preferably 5 to 10. Specific examples include allyl group, prenyl group, pentenyl group, hexenyl group, heptenyl group, octenyl group, a nonenyl group (preferably (Z)-2-nonenyl group or (E)-2-nonenyl group), and decenyl group. The alkynyl group having 2 to 10 carbon atoms may be linear or branched, and may be chain or cyclic. The number of carbon atoms is preferably 3 to 10, and more preferably 5 to 10 carbon atoms. Specific examples thereof include propargyl group, butynyl group, pentynyl group, hexynyl group, heptynyl group, octynyl group, noninyl group and a decynyl group.

The compound represented by Formula (5) may form a salt.

Examples of the salt in a basic group include salts with mineral acids such as hydrochloric acid, hydrobromic acid, nitric acid, and sulfuric acid; salts with organic carboxylic acids such as formic acid, acetic acid, citric acid, oxalic acid, fumaric acid, maleic acid, succinic acid, malic acid, tartaric acid, aspartic acid, trichloroacetic acid, and trifluoroacetic acid; and salts with sulfonic acids such as methanesulfonic acid, benzenesulfonic acid, p-toluenesulfonic acid, mesitylenesulfonic acid, and naphthalenesulfonic acid.

Among the above salts, for example, pharmacologically acceptable salts are preferable.

### <<Examples of Ionizable Lipids>>

Examples of ionizable lipids include the following lipids. Note that cKK-E12 (MD-1) and C12-200 are compounds which are not included in the above formula (5).

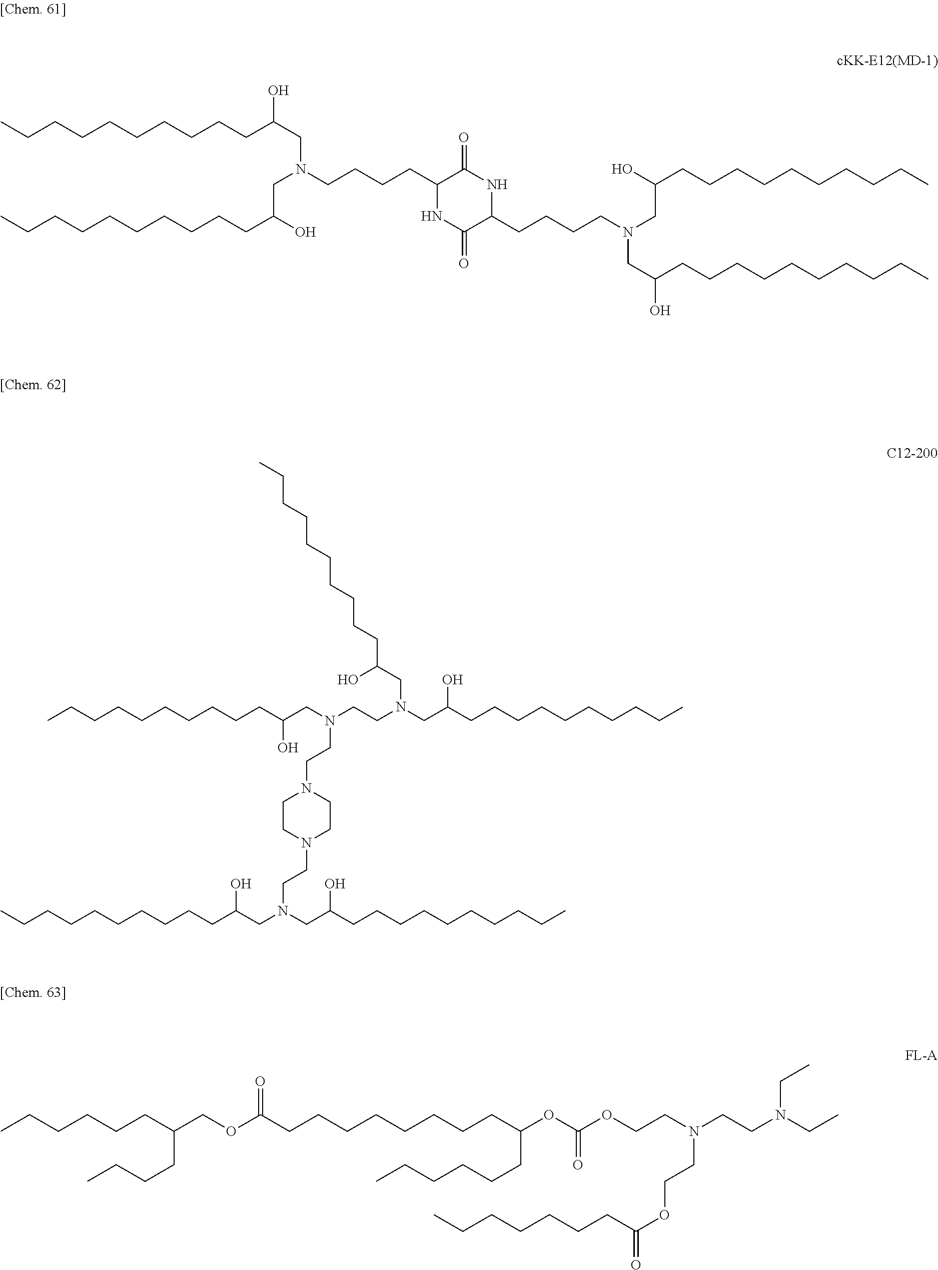

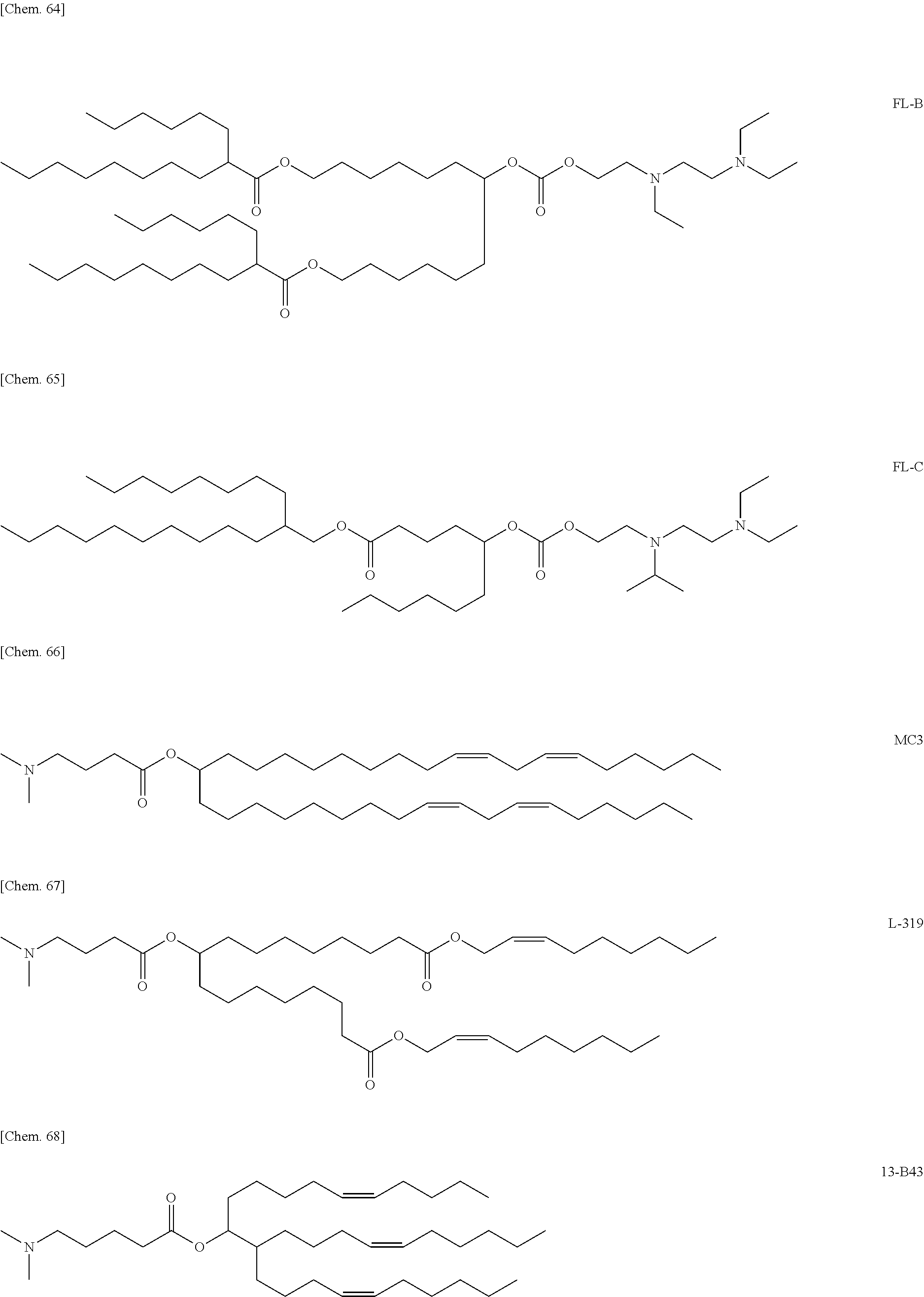

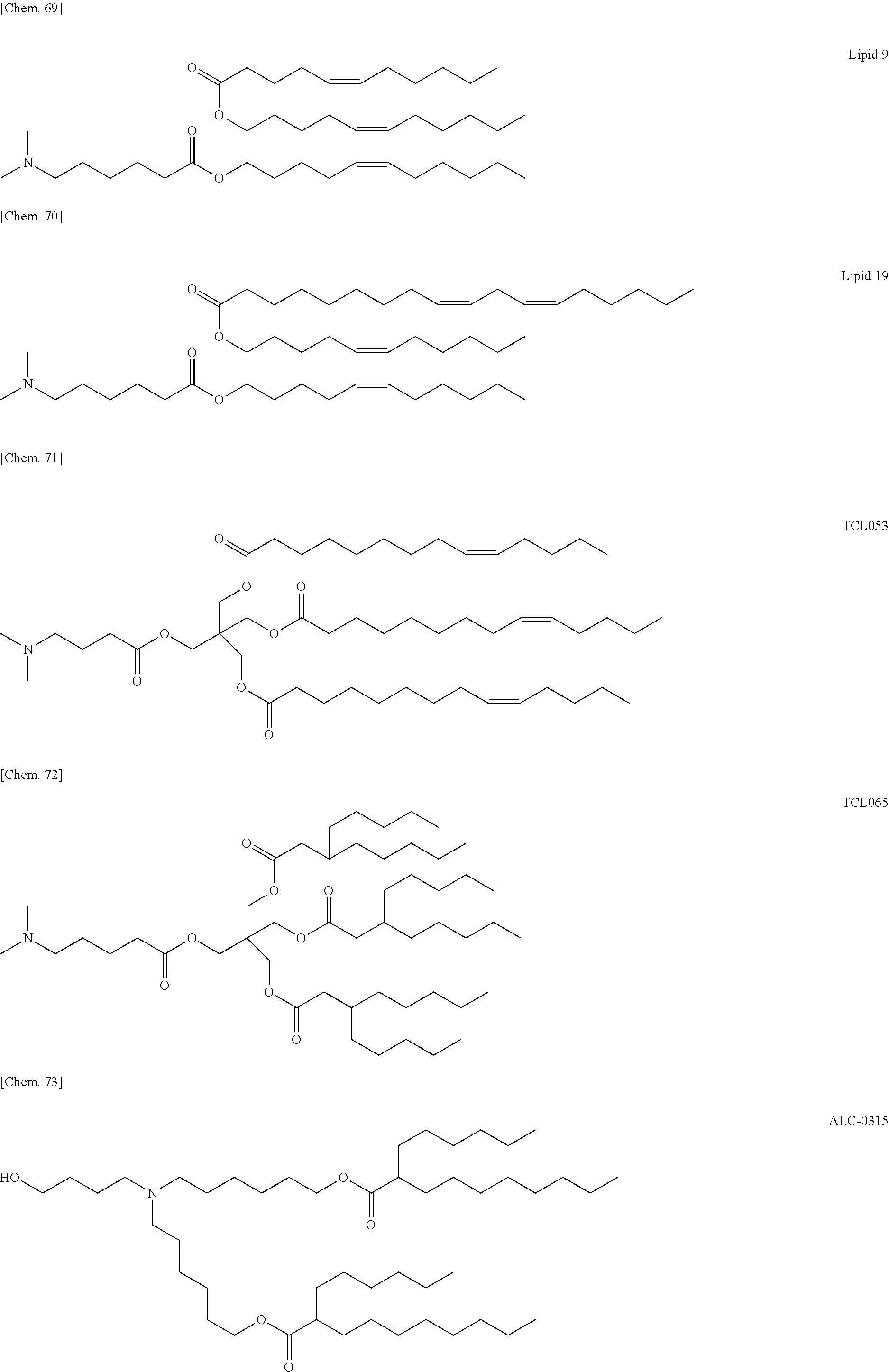

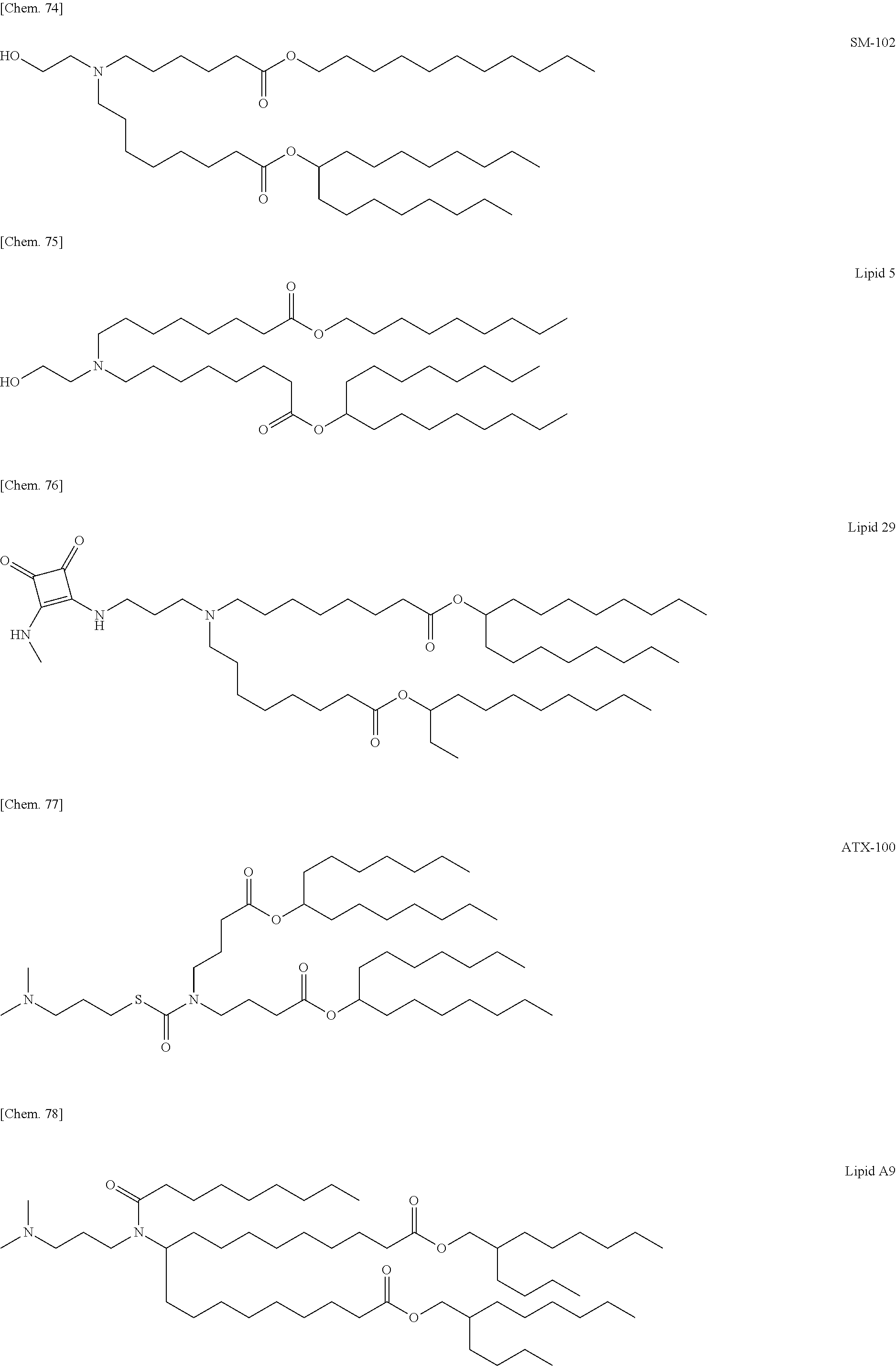

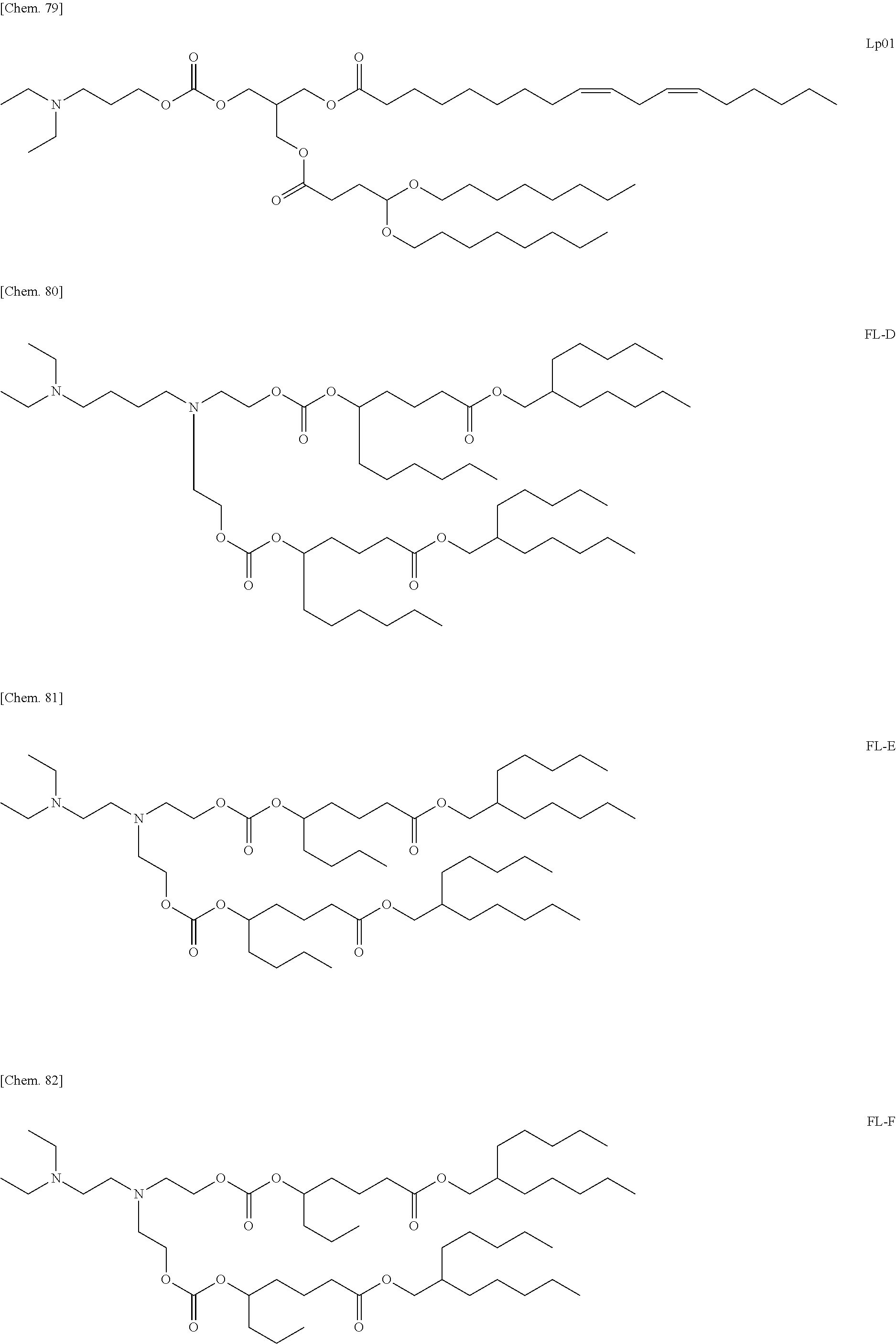

In the lipid composition of the present invention, the content of the ionizable lipid or a salt thereof with respect to the total lipids is preferably 10 mol % to 80 mol %, more preferably 20 mol % to 80 mol %, still more preferably 30 mol % to 70 mol %, further more preferably 40 mol % to 60 mol %.

### <Neutral Lipid>

The lipid particles according of the present invention may contain a neutral lipid. The neutral lipid is preferably Zwitterionic lipid.

As the zwitterionic lipid, phospholipid is preferable. Examples thereof include phosphatidylcholine, phosphatidylethanolamine, sphingomyelin, and the like. As the phospholipid, a phospholipid having a choline group such as phosphatidylcholine is preferable. The zwitterionic lipid may be used alone or in combination of a plurality of different neutral lipids.

The phosphatidylcholine is not particularly limited, and examples thereof include soybean lecithin (SPC), hydrogenated soybean lecithin (HSPC), egg yolk lecithin (EPC), hydrogenated egg yolk lecithin (HEPC), dimyristoylphosphatidylcholine (DMPC), dipalmitoylphosphatidylcholine (DPPC), distearoylphosphatidylcholine (DSPC), dioleoylphosphatidylcholine (DOPC), dilauroylphosphatidylcholine (DLPC), 1-palmitoyl-2-oleoylphosphatidylcholine (POPC), and the like. Among these, dimyristoylphosphatidylcholine (DMPC), distearoylphosphatidylcholine (DSPC) and dilauroylphosphatidylcholine (DLPC) are preferable. Particularly, distearoylphosphatidylcholine (DSPC) is preferable.
DSPC: 1,2-Distearoyl-sn-glycero-3-phosphocholine

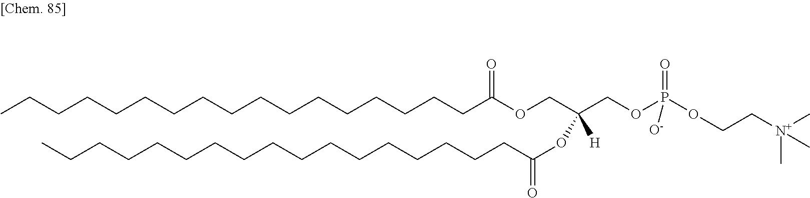 [SMILES: CCCCCCCCCCCCCCCCCC(=O)OC[C@H](COP(=O)([O-])OCC[N+](C)(C)C)OC(=O)CCCCCCCCCCCCCCCCC]

The phosphatidylethanolamine is not particularly limited, and examples thereof include dimyristoylphosphatidylethanolamine (DMPE), dipalmitoylphosphatidylethanolamine (DPPE), distearoylphosphatidylethanolamine (DSPE), dioleoylphosphatidylethanolamine (DOPE), dilinoleoylphosphatidylethanolamine (DLoPE), diphytanoylphosphatidylethanolamine (D(Phy)PE), 1-palmitoyl-2-oleoylphosphatidylethanolamine (POPE), ditetradecylphosphatidylethanolamine, dihexadecylphosphatidylethanolamine, dioctadecylphosphatidylethanolamine, diphytanylphosphatidylethanolamine, and the like.

The sphingomyelin is not particularly limited, and examples thereof include egg yolk-derived sphingomyelin, milk-derived sphingomyelin, and the like.

In the lipid composition of the present invention, the amount of the neutral lipid mixed in is preferably 1 to 30 mol %, more preferably 5 to 25 mol %, still more preferably 7 to 23 mol % with respect to the total amount of the constituent lipid components.

### <Lipid Having Nonionic Hydrophilic Polymer>

The lipid composition of the present invention may contain a lipid having a nonionic hydrophilic polymer. The lipid having nonionic hydrophilic polymer preferably contains an acyl group, and the carbon chain length of the acyl group is preferably 8 to 26.

The nonionic hydrophilic polymer is not particularly limited, and examples thereof include a nonionic vinyl-based polymer, a nonionic polyamino acid, a nonionic polyester, a nonionic polyether, a nonionic natural polymer, a nonionic modified natural polymer, and a block polymer or a graft copolymer having two or more kinds of these polymers as constitutional units.

Among these nonionic hydrophilic polymers, a nonionic polyether, a nonionic polyester, a nonionic polyamino acid, or a nonionic synthetic polypeptide is preferable, a nonionic polyether or a nonionic polyester is more preferable, a nonionic polyether or a nonionic monoalkoxy polyether is even more preferable, and polyethylene glycol (hereinafter, polyethylene glycol will be also called PEG) is particularly preferable.

The lipid having a nonionic hydrophilic polymer is not particularly limited, and examples thereof include PEG-modified phosphoethanolamine, a diacylglycerol PEG derivative, monoacylglycerol PEG derivative, a dialkylglycerol PEG derivative, a cholesterol PEG derivative, a ceramide PEG derivative, and the like. Among these, a monoacylglycerol PEG and a diacylglycerol PEG is preferable.

The alkyl chain of the lipid having a nonionic hydrophilic polymer preferably has 8 to 26 carbon atoms, and more preferably 10 to 22 carbon atoms.

The weight average molecular weight of the nonionic hydrophilic polymer is preferably 100 to 10000, more preferably 500 to 5000, and even more preferably 750 to 3000. The nonionic hydrophilic polymer chain may be branched or may have a substituent such as a hydroxymethyl group.

Preferred examples of the lipid having a nonionic hydrophilic polymer include the following lipids.
DMG-mPEG2000: 1,2-dimiristyl-rac-glycero-3-methoxypolyethylene glycol-2000
DPG-mPEG2000: 1,2-dipalmitoyl-rac-glycero-3-methoxypolyethylene glycol-2000
DSG-mPEG2000: 1,2-distearoyl-rac-glycero-3-methoxypolyethylene glycol-2000

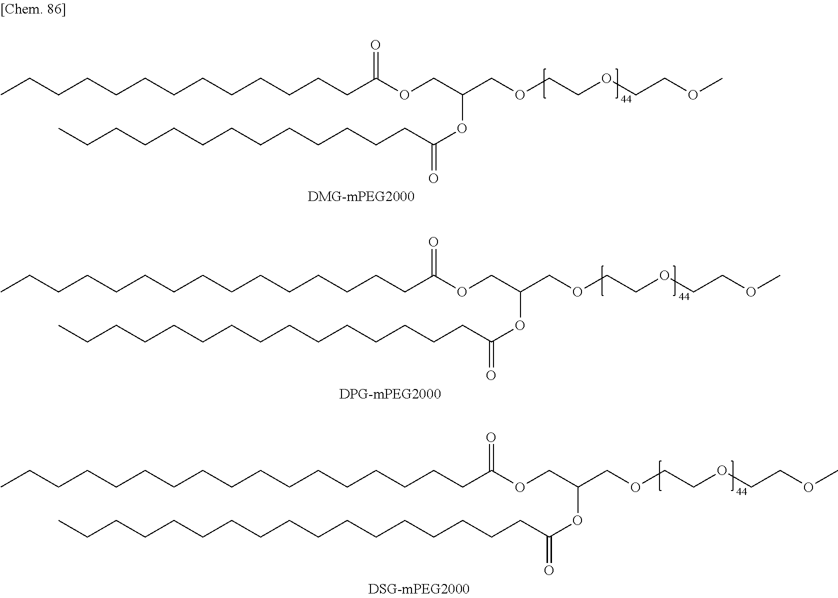

In the lipid composition of the present invention, the amount of the lipid having a nonionic hydrophilic polymer with respect to the total amount of lipids is preferably 0.1 mol % to 10 mol %, more preferably 0.3 mol % to 8 mol %, further preferably 0.5 mol % to 5 mol % and even more preferably 1 mol % to 3 mol %.

### <Nucleic Acid>

The lipid composition of the present invention contains a therapeutic agent. As the therapeutic agent, nucleic acids are preferable. The nucleic acid may be either DNA or RNA, and may be plasmid, single-stranded DNA, double-stranded DNA, siRNA (small interfering RNA), miRNA (micro RNA), mRNA, antisense oligonucleotide (also known as ASO), ribozyme, aptamer, decoy nucleic acid, gRNA used in genome editing and the like. It may also contain modified nucleic acids.

In the lipid composition of the present invention, the weight ratio of the lipid to the therapeutic agent is preferably 5 to 100, more preferably 5 to 70, still more preferably 5 to 40, and particularly preferably 5 to 35.

### <Method for Manufacturing Composition>

The method for manufacturing the lipid composition of the present invention will be described.

The method for manufacturing the lipid composition is not limited. For example, the lipid composition can be manufactured by a method in which all of the constituent components of the lipid particles or some of oil-soluble components of the lipid particles are dissolved in an organic solvent or the like such that an oil phase is formed, water-soluble components of the lipid particles are dissolved in water such that a water phase is formed, and the oil phase and the water phase are mixed together. A micromixer may be used for mixing, or an emulsifying machine such as a homogenizer, an ultrasonic emulsifying machine, or a high-pressure injection emulsifying machine may be used for emulsification.

Alternatively, the lipid composition can also be manufactured by a method in which a lipid-containing solution is subjected to evaporation to dryness using an evaporator under reduced pressure or subjected to spray drying using a spray drier such that a dried mixture containing a lipid is prepared, and the mixture is added to an aqueous solvent and further emulsified using the aforementioned emulsifying machine or the like.

One of the examples of the method for manufacturing the lipid particles containing a nucleic acid is a method including
a step (a) of dissolving the constituent components of the lipid particles containing the compound according to an embodiment of the present invention in an organic solvent so as to obtain an oil phase;
a step (b) of mixing the oil phase obtained in the step (a) with a water phase containing a nucleic acid;
a step (c) of diluting the mixed solution containing the oil phase and the water phase obtained in step (b) so as to obtain a dispersion liquid of nucleic acid-containing lipid composition; and
a step (d) of removing the organic solvent from the dispersion liquid of the nucleic acid lipid composition obtained in the step (c).

In the step (a), the lipid components are dissolved in an organic solvent (an alcohol such as ethanol, an ester, or the like). The total lipid concentration is not particularly limited, but is generally 1 mmol/L to 100 mmol/L, preferably 3 mmol/L to 50 mmol/L, and more preferably 5 mmol/L to 30 mmol/L.

In the step (b), the water phase can be obtained by dissolving a nucleic acid (for example, siRNA, an antisense nucleic acid, mRNA or the like) in water or a buffer. If necessary, a component such as an antioxidant can be added. The mixing ratio (volume ratio) of water phase:oil phase is preferably 5:1 to 1:1 and more preferably 4:1 to 2:1.

In the step (b), the mixed solution can be diluted with water or a buffer (for example, phosphate buffered saline (PBS) or the like).

In the step (c), as the method of removing the organic solvent from the dispersion liquid of the lipid composition, a general method can be used without particular limitation. For example, by dialyzing the dispersion liquid with the phosphate buffered saline, the organic solvent can be removed.

If necessary, the lipid composition can be subjected to sizing. Although the sizing method is not particularly limited, an extruder or the like can be used to reduce the particle size.

### <Composition>

The composition of the present invention may be lipid particle. The lipid particle means a particle composed of a lipid, and includes a composition having any structure selected from a lipid aggregate (for example, lipid nanoparticles) in which the lipid is aggregated. a micelle, and a liposome. However, the structure of the lipid particles is not limited to these as long as the composition contains lipids.

The form of the lipid particles can be checked by electron microscopy, structural analysis using X-rays, and the like. For example, by a method using Cryo transmission electron microscopy (CryoTEM method), it is possible to check, for example, whether a lipid particle such as a liposome has a structure composed of a bimolecular lipid membrane structure (lamella structure) and an inner water layer or a structure composed of an inner core with a high electron density and packed with constituent components including a lipid. The X-ray small angle scattering (SAXS) analysis also makes it possible to check whether or not a lipid particle has a bimolecular lipid membrane structure (lamella structure).

When the lipid composition of the present invention is a particle, the particle size is not particularly limited, but is preferably 10 to 1,000 nm, more preferably 30 to 500 nm, and even more preferably 50 to 250 nm. The particle size of the lipid particles can be measured by a general method (for example, a dynamic light scattering method, a laser diffraction method, or the like).

When the lipid composition of the present invention is a particle, the zeta potential of the particle is not particularly limited, but is preferably −20 to +20 mV, and more preferably −10 to 10 mV. The zeta potential in the present invention is a value measured by the electrophoresis method obtained by diluting the lipid composition in a phosphate buffer solution, but the method is not limited thereto.

The pKa of the lipid composition of the present invention is not particularly limited, but is preferably 9 to 4, more preferably 8 to 5, and even more preferably 7.5 to 6. The pKa of the lipid composition in the present invention adopts the value measured by the TNS assay, but is not limited to this.

### <Use of Lipid Composition>

As an example of the use of the lipid composition in the present invention, a therapeutic agent (for example, nucleic acid) can be introduced into the cell by introducing the lipid composition containing nucleic acid into the cell. That is, the lipid composition of the present invention can be used as a composition for introducing nucleic acid into cells. Further, the lipid composition of the present invention can be used as a pharmaceutical composition for nucleic acid delivery in vivo.

In the present invention, in particular, the therapeutic agent can be delivered to the endothelial cells, mesenchymal cells, or cancer cells. Therefore, a therapeutic agent can be delivered to organs other than the liver. Organs other than the liver include spleen, kidney, lung, heart, muscle, and brain. The organs other than the liver to which the lipid composition of the present invention delivers the therapeutic agent are preferably spleen, kidney, lung, heart, muscle, and brain, more preferably kidney, lung, heart, muscle, and brain, and even more preferably lung and heart.

Further, when the lipid composition of the present invention contains a nucleic acid having a medicinal use, the lipid composition can be administered to a living body as a nucleic acid medicine. When the lipid composition of the present invention is used as a nucleic acid drug, the lipid composition of the present invention alone may be administered to a living body, or the lipid composition may be mixed with a pharmaceutically acceptable carrier (eg, an administration medium such as saline or phosphate buffer) and administered to a living body. That is, the lipid composition of the present invention may further contain a pharmaceutically acceptable carrier.

The concentration of the lipid composition in the mixture with the pharmaceutically acceptable carrier is not particularly limited and can generally be 0.05% by weight to 90% by weight. Further, other pharmaceutically acceptable additives such as a pH adjustment buffer and an osmotic pressure adjustment agent may be added to the nucleic acid drug containing the lipid composition of the present invention.

The route of administration for administering the lipid composition of the present invention is not particularly limited. The lipid composition can be administered by any method. Examples of the administration method include oral administration and parenteral administration (intra-articular administration, intravenous administration, intra-arterial administration, subcutaneous administration, intracutaneous administration, intravitreal administration, intraperitoneal administration, intramuscular administration, intravaginal administration, intravesical administration, intrathecal administration, pulmonary administration, rectal administration, colonic administration, buccal administration, nasal administration, intracisternal administration, inhalation, and the like). Among these, parenteral administration is preferable. As the method of administration, intravenous injection, subcutaneous injection, intracutaneous injection, or intramuscular injection is preferable. Intravenous injection or intramuscular injection is particularly preferable. As the administration, nucleic acid delivery can also be performed by local administration in vivo. The lipid composition of the present invention can also be administered by direct injection into the diseased site.

The dosage form of the lipid particles according to the embodiment of the present invention is not particularly limited. For oral administration, the lipid composition of the present invention can be used in the form of tablets, troches, capsules, pills, suspension, syrup, and the like by being combined with an appropriate excipient. In addition, additives such as an antioxidant, a buffer, a bacteriostat, an isotonic sterile injection, a suspending agent, a solubilizer, a thickener, a stabilizer, and a preservative can be appropriately incorporated into formulations suitable for parenteral administration.

### <Use of Lipid Nanoparticle as Nucleic Acid Delivery Carrier>

The lipid particles in the present invention can retain a nucleic acid at a high encapsulation rate. Therefore, the lipid particles are extremely useful as a nucleic acid delivery carrier. According to the nucleic acid delivery carrier using the present invention, for example, by mixing the obtained lipid particles with a nucleic acid or the like and performing transfection in vitro or in vivo, the nucleic acid and the like can be introduced into cells. Furthermore, the nucleic acid delivery carrier using the present invention is also useful as a nucleic acid delivery carrier in nucleic acid drugs. That is, the lipid particles according to the embodiment of the present invention are useful as a composition for in vitro or in vivo (preferably in vivo) delivery of a nucleic acid.

Next, the present invention will be described based on examples, but the present invention is not limited thereto.

### EXAMPLE

### Materials and Methods

<siRNA>

The following custom siRNA was manufactured by Horizon.
siRNA against murine VECadherin (siVEcad, siCdh5)

Sense:
5′-mCmCAAAAGAGAGAmCmUGGAmUmUdTsdT-3′
Antisense:
5′-AAUCmCAGUCUCUCUUUUGGdTsdT-3′

### Abbreviation

A Adenosine-3′-phosphate
C Cytidine-3′-phosphate
G Guanonsine-3′-phosphate
U Uridine-3′-phosphate
mA 2′-O-methyladenosine-3′-phosphate
mC 2′-O-methylcytidine-3′-phosphate
mG 2′-O-methylguanonsine-3′-phosphate
mU 2′-O-methyluridine-3′-phosphate
dT 2′-deoxythymidine-3′-phosphate
dTs 2′-deoxythymidine-5′-phosphate-phosphorothioate
Cadherin 5 (Cdh5), also known as vascular endothelial cadherin (VE-cadherin) is a junctional protein whose expression is limited to endothelia cells. By quantifying Cdh5 mRNA remaining after siCdh5 administration, delivery efficiency to endothelial cells can be evaluated.

### <Lipid Nanoparticle Formulation>

All chemicals obtained from commercial sources were stored following the manufacturer's note and used without further purification.

Lipid Nanoparticles were synthesized using a microfluidics chip device as previously described. Lipid Nanoparticles were formed by mixing a lipid-containing ethanol phase with a siRNA-containing aqueous phase, and pumped through microfluidic channel in the PDMS (poly-dimethyl-siloxane) chip.

Ionizable lipids, 1,2-distearoyl-sn-glycero-3-phosphocholine (DSPC, NOF), cholesterol (Sigma) and 1,2-Dimyristoyl-rac-glycero-3-methylpolyoxyethylene (DMG-mPEG2000, NOF) were dissolved in ethanol and mixed at a predetermined molar ratio.

The aqueous phase was prepared in 10 mM citrate buffer (pH 3.0, fisher) with siRNA. The ethanol and aqueous phases were mixed at a 3:1 ratio in a microfluidic chip device using syringe pumps as previously described. Total flow rate was 1.2 mL/min.

The lipid nanoparticles were dialyzed against 1×PBS in a 20,000 MWCO cassette (Invitrogen) at 4° C. overnight. Formulations were concentrated using Amicon ultra centrifugal filters (Millipore Sigma), and sterilized through a 0.22 um filter (Millipore Sigma), and stored at 4° C.

### <Lipid Nanoparticle Characterization>

The nucleic acid encapsulation efficiency of lipid nanoparticles was calculated by using a modified QUANT-IT™ RIBOGREEN™ RNA assay (Invitrogen Corporation Carlsbad, Calif.) in accordance with the method of Walsh C. et al. Methods Mol Biol. 2014; 1141:109-20. Briefly, the samples were diluted to a concentration of approximately 1.5 μg/ml in a TE buffer. 50 μl of the diluted samples were transferred to a black U-bottom 96 well plate and either 50 μl of TE buffer or 50 μl of a 2% Triton X-100 containing TE buffer was added to the wells. The plate was incubated at a temperature of 37° C. for 15 minutes. The RIBOGREEN™ reagent was diluted 1:200 in TE buffer, and 100 μl of this solution was added to each well. The fluorescence intensity was measured using a plate reader (Tecan 200 Pro, Tecan) at an excitation wavelength of 485 nm and an emission wavelength of 515 nm. The fluorescence values of the reagent blank were subtracted from that of each of the samples and the percentage of free RNA was determined by dividing the fluorescence intensity of the intact sample (without Triton X-100) by the fluorescence value of the disrupted sample (with Triton X-100).

A Zetasizer Nano ZS (Malvern Instruments) was used to determine the particle size, the polydispersity index (PDI) and the zeta potential of the nanoparticle compositions in 1×PBS in determining particle size and 0.1×PBS in determining zeta potential.

### <pKa Value Measurement by TNS Assay>

The apparent pKa values of lipid nanoparticles were determined using TNS assay in accordance with the method of Heyes J. et al. Journal of Controlled Release 107 (2005) 276-287. Briefly, TNS was prepared as a 100 M stock solution in DMSO. Lipid nanoparticles were diluted to 25 ng/mL ionizable lipid in 0.1×PBS. Furthermore, this lipid nanoparticle solution was diluted 10-fold in a buffer solution having a pH in the range of about 2.5 to 9.0. To this lipid nanoparticle solution with different pH was added 1TNS stock solution to a final concentration of 6 μM, and was mixed well in a black-384 well plate. Fluorescence intensity was monitored in a Tecan Pro200 plate reader using excitation and emission wavelengths of 320 nm and 465 nm. With the resulting fluorescence values, a sigmoidal plot of fluorescence versus buffer pH was created and the log of the inflection point of this curve was the apparent pKa of the lipid nanoparticle formulation. pKa values were obtained using Prism Software.

### <Animal Experiments>

All animal studies were approved by the MIT Institutional Animal Care and Use Committee (CAC) and were consistent with local, state, and federal regulations as applicable. All experimental procedures were performed with ethical compliance and approval under the guidelines for Division of Comparative Medicine by Massachusetts Institute of Technology. Female C57BL/6 mice (6 weeks) were obtained from Jackson Laboratory, housed in an MIT animal facility, and acclimated for at least 3 days before the initiation of a study.

For intravenous administration, siRNA-lipid nanoparticles diluted in PBS were injected via the tail vein using 29 g, 3/10 cc insulin syringes (BD Biosciences) after gentle warming of the animals using a heat lamp. For intramuscular administration, siRNA-lipid nanoparticles diluted in PBS were injected via the tail vein using 29 g, 3/10 cc insulin syringes (BD Biosciences).

48-72 hours after injection, organs or tissues including the heart, liver, spleen, lung, kidneys, muscle (quadriceps and diaphragm), brain and tumors were collected and soaked in RNAlater solution for 12-48 hrs at 4 degC. and stored at −20 degC. after the removal of RNAlater.

<Tissue mRNA Quantification by qPCR>

Total RNA was isolated from tissues using the TRIzol. Briefly, tissue punches were placed in a deep-well 96 well plate with 4-mm stainless beads and lysed with 350 μL TRIzol using a GenoGrinder2010. 300 μL of lysate was then transferred to a new deep-well 96 well plate. Total RNA was further purified using Direct-zol-96 MagBead (Zymo Rsearche) according to the manufacturer's protocol.

Gene expression was analyzed in a one-step multiplex qPCR with Luna™ Universal Probe One-Step RT-qPCR Kit (NEB) and Taqman probes-Cdh5 Mm00486938_m1), Gusb (Mm01197698_m1) and B2m (Mm00437762_m1). Samples were amplified using a LightCycler 480 qPCR machine (Roche). Cdh5 expression was normalized to B2m or Gusb.

### Example 1: DC-Cholesterol Incorporation

To examine the potential of synthetic cholesterol analogues with a basic functional group, we first replaced cholesterol in standard hepatocyte-targeting LNPs with DC-cholesterol. DC-cholesterol was originally developed for nucleic acid delivery liposomes and has an ionizable tertiary amine group of pKa of 7.8 in cholesterol C3-position(Non-patent documents 11 and 12). Although combination of ionizable lipid and DC-cholesterol was tested in subcutaneous mRNA vaccines for enhanced mRNA delivery to dendritic cells in lymph node, it didn't show any advantage over cholesterol, or even reduced the delivery efficiency.

LNP1 is a standard hepatocyte-targeting LNP formulation including about 50 mol % ionizable lipid, about 10 mol % DSPC, about 38.5 mol % cholesterol, and about 1.5 mol % PEG-DMG. This type of formulation is used in the US Food and Drug Administration-approved Onpattro™ and Spikevax™. LNP2 replaced cholesterol with DC-cholesterol at the same lipid ratio with LNP1.

TABLE 1
Physicochemical properties of cholesterol and DC-cholesterol LNPs

| | | | | | | Zeta |
|---|---|---|---|---|---|---|
| | Ionizable | | Encapsulation | Z-average | | potential |
| | lipid | sterol | (%) | (nm) | pKa | (mV) |
| LNP1 | FL-A | Cholesterol | 77 | 98.4 | 6.72 | −5.6 |
| LNP2-1 | FL-A | DC-cholesterol | 83 | 87.4 | 6.86 | 1.3 |

To evaluate gene silencing in endothelial cells in various organs, we used siRNA against Cdh5, a cell adhesion molecule whose expression is limited to endothelium. Formulations including an siRNA against Cdh5 were intravenously administered to mice at 0.3 mg/kg and organs were harvested 48 hours after injection. After isolation and purification of total RNA from organs, Cdh5 mRNA was quantified relative to a housekeeping gene, B2m. A PBS control was also tested.

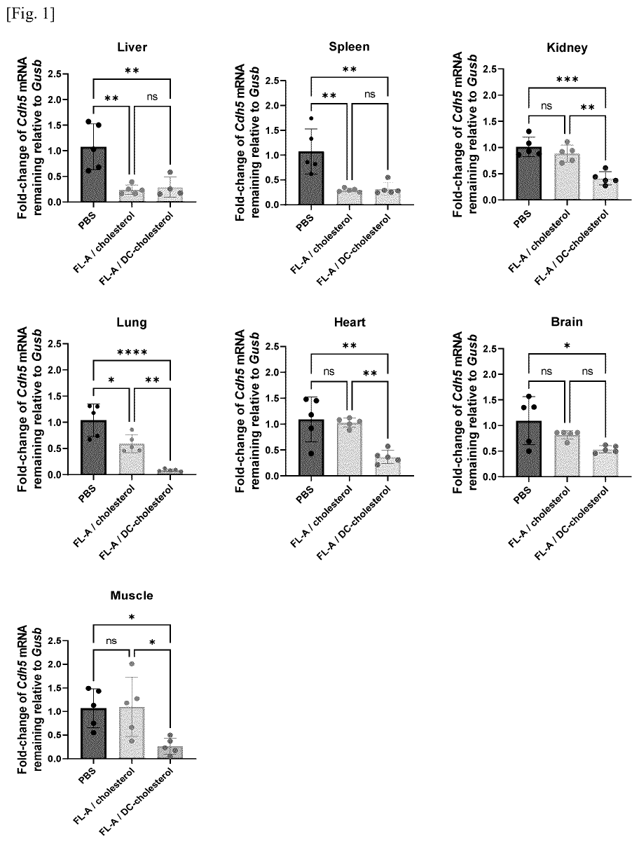

As shown in FIG. 1, by substituting DC-cholesterol for cholesterol in conventional LNP has enabled silencing of endothelial genes in various organs of mice. VE-cadherin mRNA expression was measured based on ΔΔCt calculations compared to PBS 3 days post-injection. (0.3 mg/kg siVEcad)

### Example 2 Generalization to Other Ionizable Lipids

To test the generalizability of the DC-cholesterol LNP formulation, we tested different types of ionizable lipids. Table 2 summarizes the physicochemical properties of tested formulations. FL-A, FL-B and FL-C were developed by FUJIFILM.

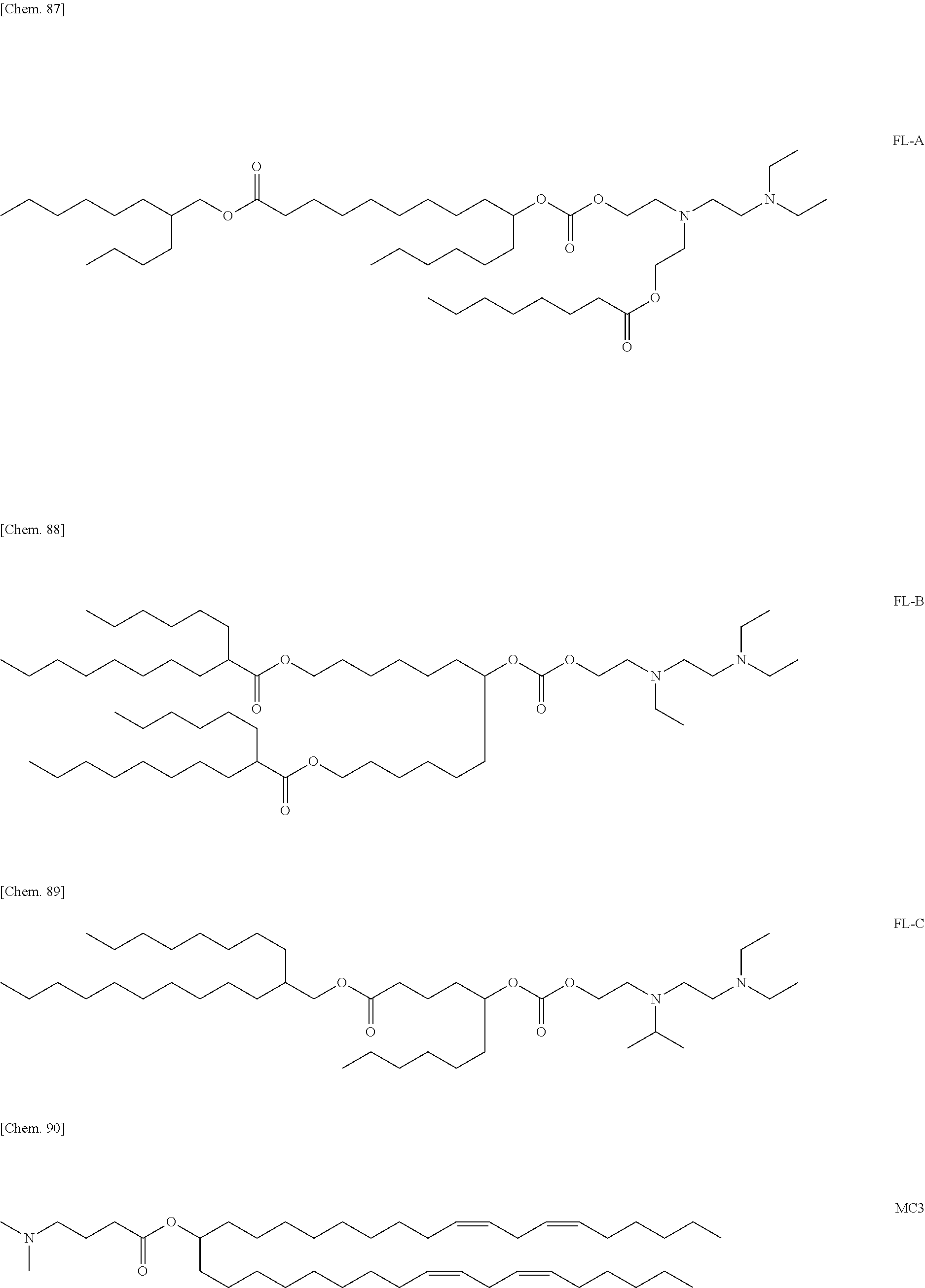

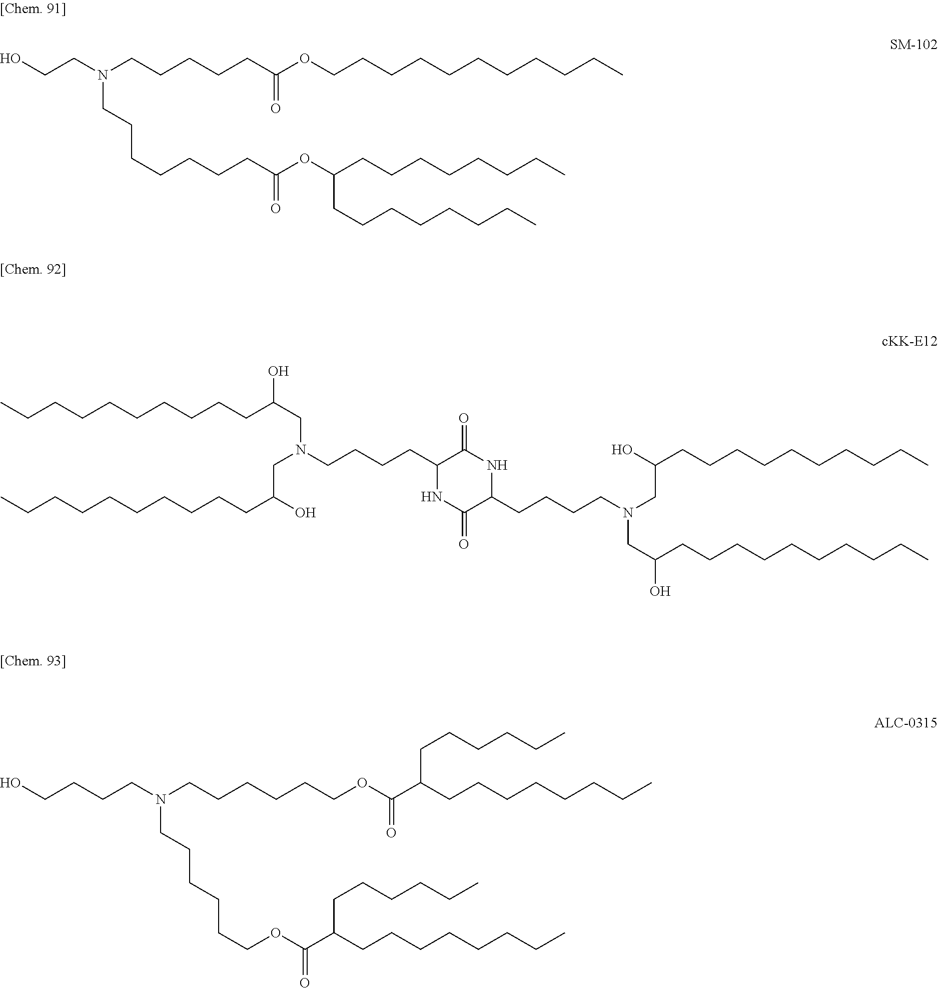

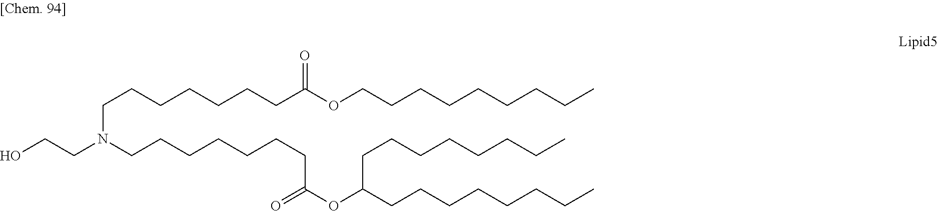 [SMILES: CCCCCCCCCOC(=O)CCCCCCCN(CCO)CCCCCCCC(=O)OC(CCCCCCCC)CCCCCCCC]

TABLE 2
Physicochemical propeties of DC-cholesterol LNPs with different ionizalbe lipids

| | | Ionizable | | | | Zeta |
|---|---|---|---|---|---|---|
| | Ionizable | lipid:phospholipid:sterol:PEG- | Encapsulation | Z-average | | potential |
| | lipid | lipid | (%) | (nm) | pKa | (mV) |
| LNP2-2 | FL-A | 50:10:38.5:1.5 | 84 | 74.3 | 6.86 | 1.3 |
| LNP3 | FL-B | 50:10:38.5:1.5 | 80 | 69.3 | 6.38 | −4.4 |
| LNP4 | FL-C | 50:10:38.5:1.5 | 89 | 61.5 | 6.62 | 6.4 |
| LNP5 | MC3 | 50:10:38.5:1.5 | 92 | 65.8 | 6.91 | −0.1 |
| LNP6 | SM-102 | 50:10:38.5:1.5 | 91 | 56.5 | 7.20 | 7.7 |
| LNP7 | cKK-E12 | 50:10:38.5:1.5 | 76 | 84.2 | 5.61 | −1.9 |
| LNP8 | ALC-0315 | 50:10:38.5:1.5 | 92 | 58.3 | 6.60 | 7.2 |
| LNP9 | Lipid-5 | 50:10:38.5:1.5 | 92 | 69.1 | 7.05 | 8.2 |
| LNP10 | — | 0:60:38.5:1.5 | 75 | 119.4 | 7.19 | 10.5 |

Formulations including an siRNA against Cdh5 were intravenously administered to mice at 0.5 mg/kg and organs were harvested 72 hours after injection. After isolation and purification of total RNA from organs, Cdh5 mRNA was quantified relative to a housekeeping gene, Gusb. A PBS control was also tested.

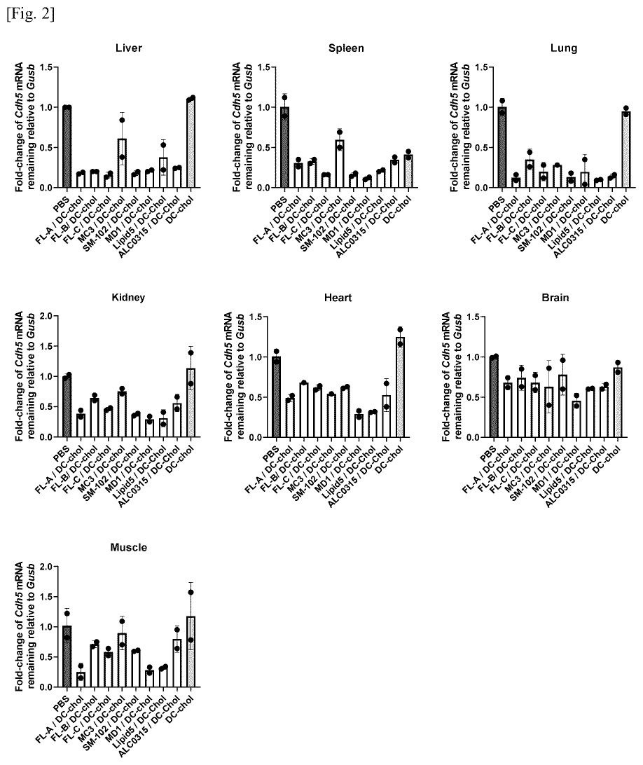

The results are shown in FIG. From FIG. 2, silencing of endothelial genes with various ionizable lipids was demonstrated by substitution with DC-cholesterol. Cdh5 mRNA expression was measured based on ΔΔCt calculations compared to PBS 3 days post-injection. (0.5 mg/kg siCdh5)

### Example 3: Evaluation of Other C3-Position Modified Cholesterol Derivatives

To elucidate structural requirement of cholesterol derivatives, a range of them were formulated in LNPs. The lipid composition of LNPs were fixed as FL-A:DSPC:sterol DMG-mPEG2000=50:10:38.5:1.5.

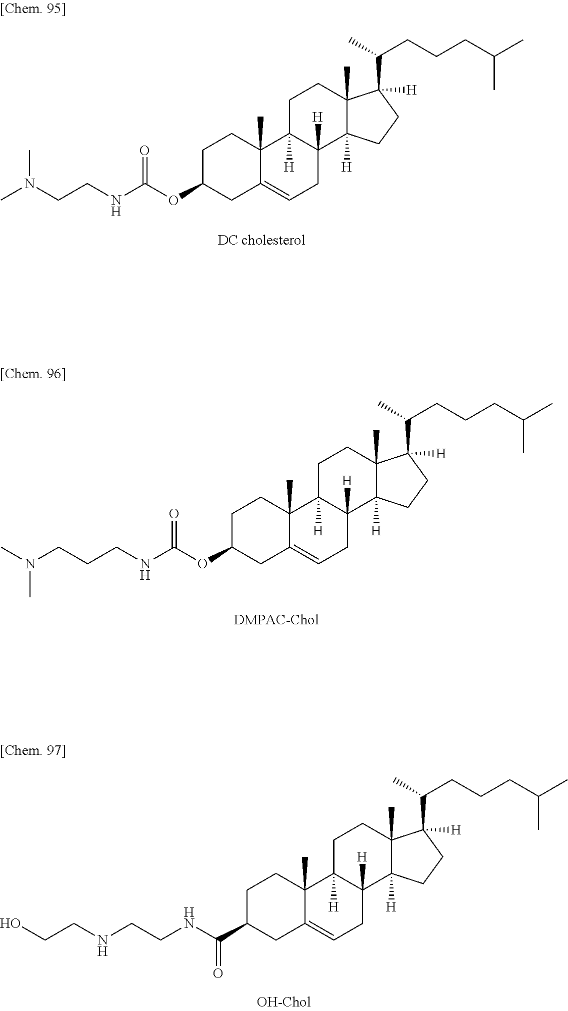

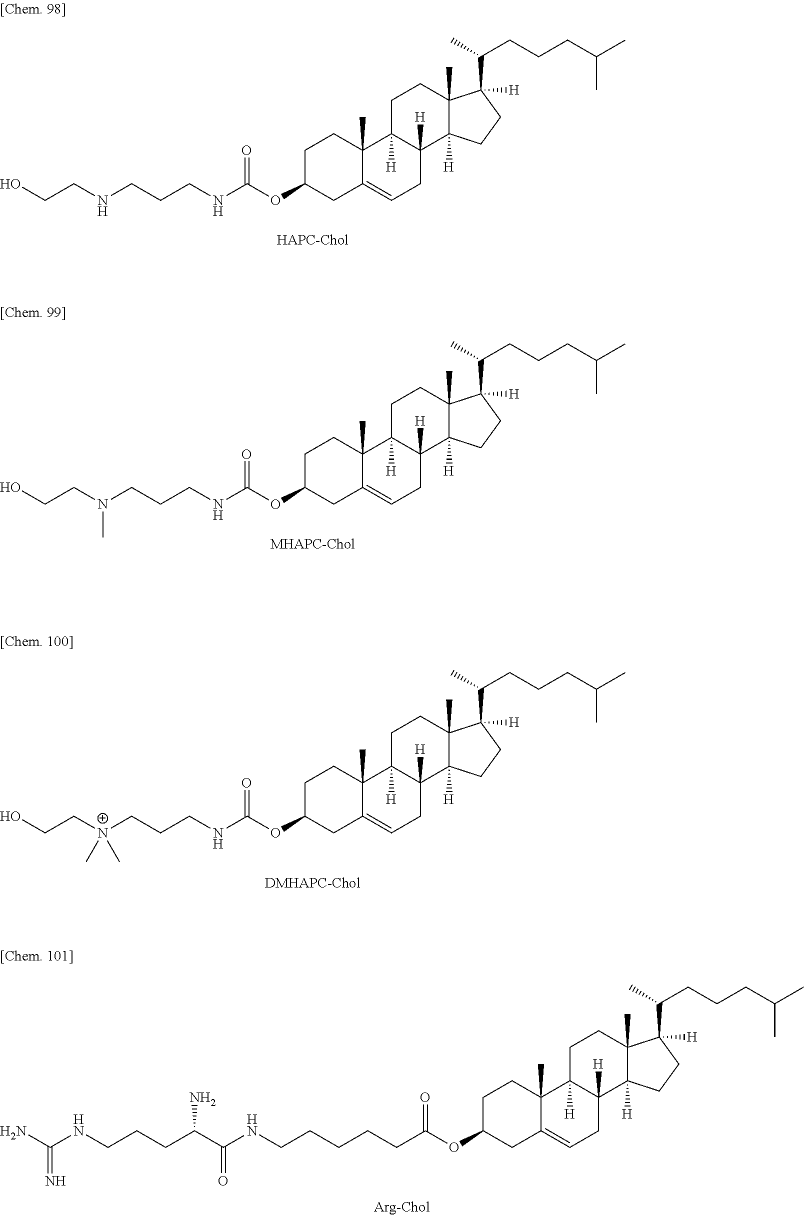

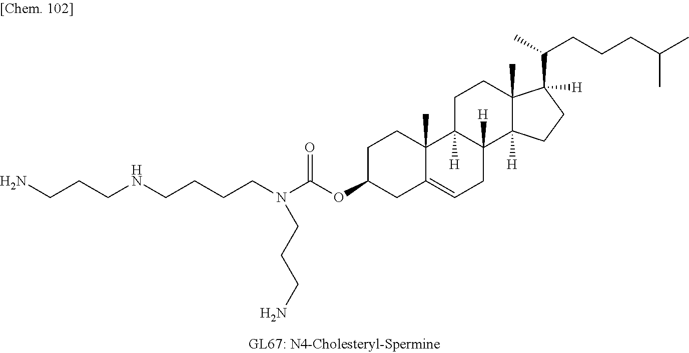

TABLE 3
Physicochemical propeties of 3′-modihied cholesterol LNPs

| | | | | | | Zeta |
|---|---|---|---|---|---|---|
| | Ionizable | | Encapsulation | Z-average | | potential |
| | lipid | Sterol | (%) | (nm) | pKa | (mV) |
| LNP2-3 | FL-A | DC-cholesterol | 84 | 74.3 | 6.71 | 4.0 |
| LNP11 | FL-A | DMPAC-Chol | 80 | 69.3 | 6.78 | 6.2 |
| LNP12 | FL-A | OH-Chol | 89 | 61.5 | 6.66 | 8.0 |
| LNP13 | FL-A | HAPC-Chol | 92 | 65.8 | 6.90 | 7.2 |
| LNP14 | FL-A | MHAPC-Chol | 91 | 56.5 | 6.83 | 2.4 |
| LNP15 | FL-A | DMHAPC-Chol | 76 | 84.2 | 6.71 | 5.9 |
| LNP16 | FL-A | Arg-Chol | 92 | 58.3 | 6.36 | 8.9 |
| LNP17 | FL-A | GL-67 | 100 | 72.1 | 7.80 | 13.0 |

Formulations including an siRNA against Cdh5 were intravenously administered to mice at 0.5 mg/kg and organs were harvested 72 hours after injection. After isolation and purification of total RNA from organs, Cdh5 mRNA is quantified relative to a housekeeping gene, Gusb. A PBS control was also tested.

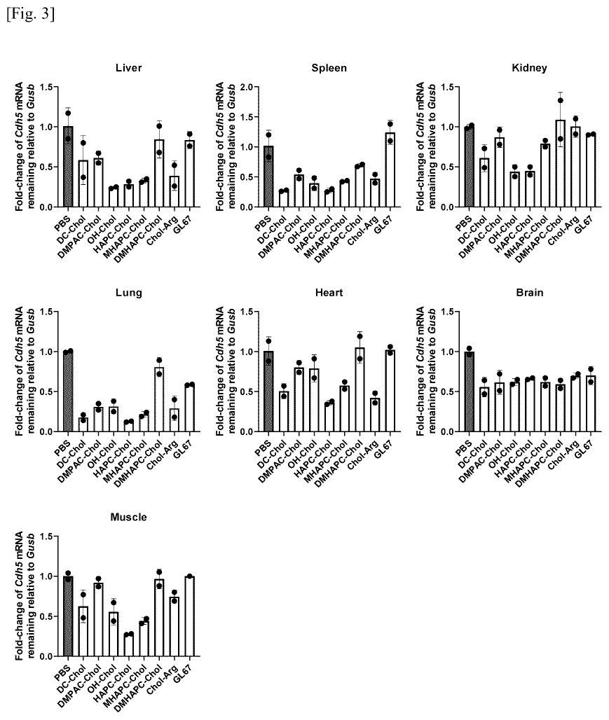

The results are shown in FIG. 3. As shown in FIG. 3, various 3′modified cholesterol analogs showed silencing of endothelial genes in various organs of mice. Cdh5 mRNA expression was measured based on ΔΔCt calculations compared to PBS 3 days post-injection. (0.5 mg/kg siCdh5)

### Example 4: DC-Cholesterol Ratio (FL-B)

To optimize the DC-cholesterol ratio, we prepared LNP formulations with different DC-cholesterol ratio and tested them in vivo. In this example, FL-A was used for the ionizable lipid, and DSPC and DMG-mPEG-2000 ratio was fixed as 10% and 1.5%, respectively.

Table 4 summarizes the content and characteristics of several formulations of lipid components useful for nanoparticle compositions of the invention.

TABLE 4
Physicochemical properties of DC-cholesterol LNPs with different molar ratio

| | Total lipid/RNA | Ionizable | Encapsulation | Z-average | |
|---|---|---|---|---|---|
| | Weight ratio | lipid:phospholipid:Sterol:PEG-lipid | (%) | (nm) | pKa |
| LNP18 | 17.1 | 10:10:78.5:1.5 | 98 | 121.3 | 7.156 |
| LNP19 | 17.0 | 20:10:68.5:1.5 | 98 | 60.7 | 7.156 |
| LNP20 | 17.1 | 30:10:58.5:1.5 | 98 | 56.8 | 6.477 |
| LNP21 | 17.0 | 40:10:48.5:1.5 | 90 | 64.8 | 6.575 |
| LNP22 | 17.0 | 50:10:38.5:1.5 | 81 | 78.8 | 6.543 |
| LNP23 | 17.1 | 60:10:28.5:1.5 | 80 | 74.0 | 6.316 |
| LNP24 | 17.1 | 70:10:18.5:1.5 | 69 | 83.9 | 6.386 |
| LNP25 | 17.0 | 78.5:10:10:1.5 | 51 | 138.8 | 6.392 |

Formulations including an siRNA against Cdh5 were intravenously administered to mice at 0.5 mg/kg and organs were harvested 72 hours after injection. After isolation and purification of total RNA from organs, Cdh5 mRNA is quantified relative to a housekeeping gene, B2m. APBS control was also tested.

The results are shown in FIG. 4. As shown in FIG. 4, LNPs with different DC-cholesterol ratios showed strong silencing of endothelial genes in lung and heart in mice. Cdh5 mRNA expression was measured based on ΔΔCt calculations compared to PBS 3 days post-injection. (0.5 mg/kg siCdh5)

### Example 5: DC-Cholesterol Ratio 2 (FL-A)

To optimize the DC-cholesterol ratio, we prepared LNP formulations with different DC-cholesterol ratio and test them in vivo. In this example, FL-A was used for the ionizable lipid, and DSPC and DMG-mPEG-2000 ratio was fixed as 10% and 1.5%, respectively.

Table 5 summarizes the content and characteristics of several formulations of lipid components useful for nanoparticle compositions of the invention.

TABLE 5
Physicochemical properties of DC-cholesterol LNPs with different molar ratio

| | | Ionizable | | | | |
|---|---|---|---|---|---|---|
| | | lipid:phospho- | | | | Zeta |
| | | lipid:Sterol:PEG- | Encapsulation | Z-average | | potential |
| | Sterol | lipid | (%) | (nm) | pKa | (mV) |
| LNP26 | DC-cholesterol | 10:10:78.5:1.5 | 98 | 78.6 | 7.29 | 13.9 |
| LNP27 | DC-cholesterol | 20:10:68.5:1.5 | 94 | 70.1 | 7.00 | 9.1 |
| LNP28 | DC-cholesterol | 30:10:58.5:1.5 | 94 | 67.1 | 7.04 | 9.4 |
| LNP29 | DC-cholesterol | 40:10:48.5:1.5 | 91 | 76.3 | 6.85 | 10.0 |
| LNP2-4 | DC-cholesterol | 50:10:38.5:1.5 | 84 | 57.6 | 6.71 | 4.0 |
| LNP30 | DC-cholesterol | 60:10:28.5:1.5 | 79 | 69.2 | 6.84 | 5.3 |
| LNP31 | DC-cholesterol | 70:10:18.5:1.5 | 72 | 67.1 | 6.82 | 0.6 |
| LNP32 | DC-cholesterol | 80:10:8.5:1.5 | 43 | 69.1 | 6.67 | −3.7 |

Formulations including an siRNA against Cdh5 were intravenously administered to mice at 0.5 mg/kg and organs were harvested 72 hours after injection. After isolation and purification of total RNA from organs, Cdh5 mRNA is quantified relative to a housekeeping gene, Gusb. APBS control was also tested.

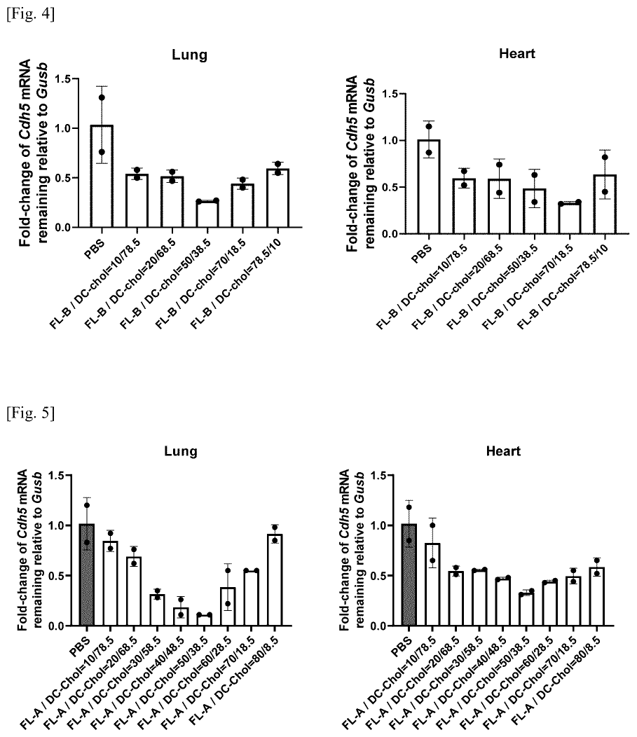

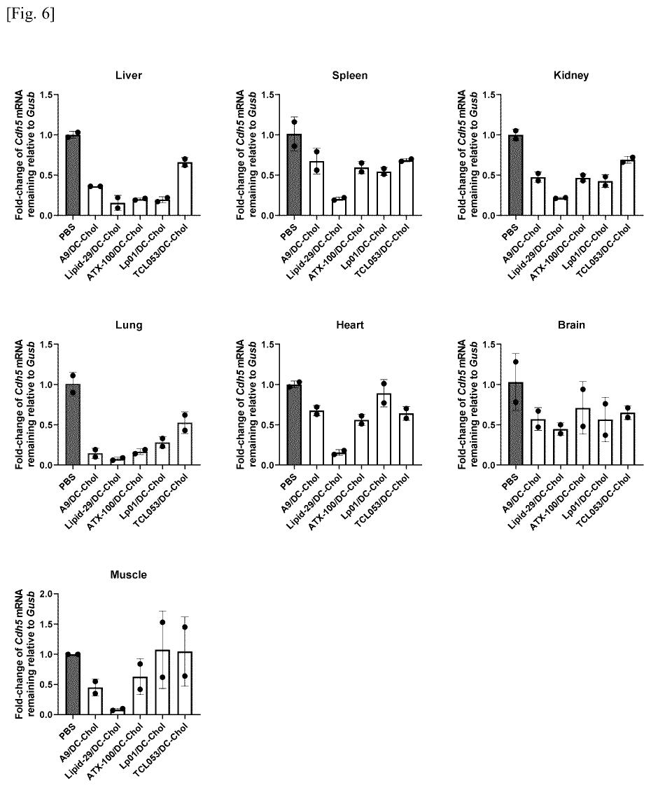

The results are shown in FIG. 5. As shown in FIG. 5, LNPs with different DC-cholesterol ratios showed strong silencing of endothelial genes in lung and heart in mice. Cdh5 mRNA expression was measured based on ΔΔCt calculations compared to PBS 3 days post-injection (0.5 mg/kg siCdh5).

### Example 6: Generalization to Other Ionizable Lipids

To further test the generalizability of the DC-cholesterol LNP formulation, we tested different types of ionizable lipids. Table 6 summarizes the physicochemical properties of tested formulations.

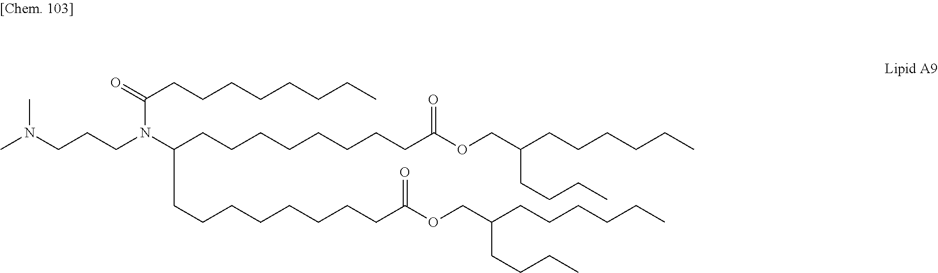 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

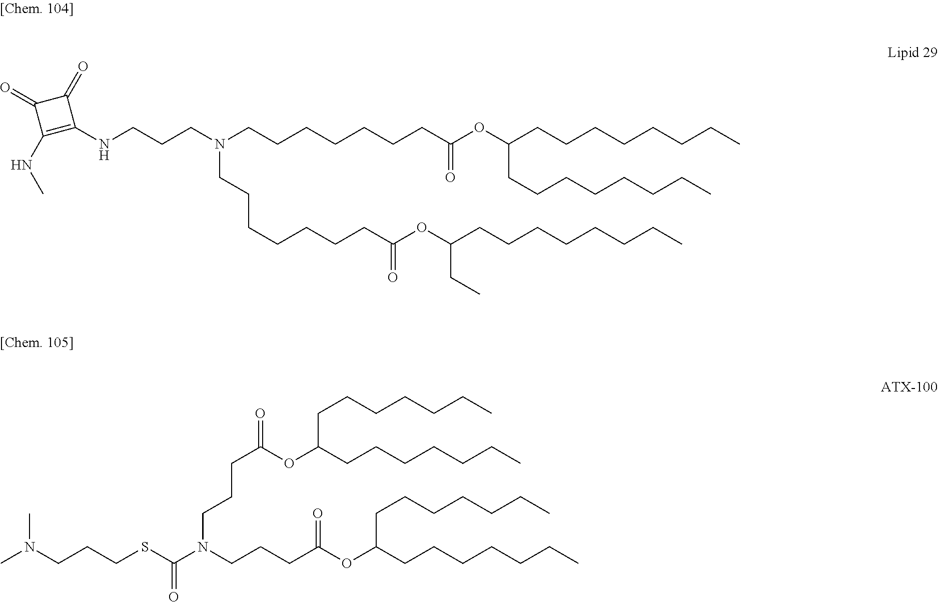

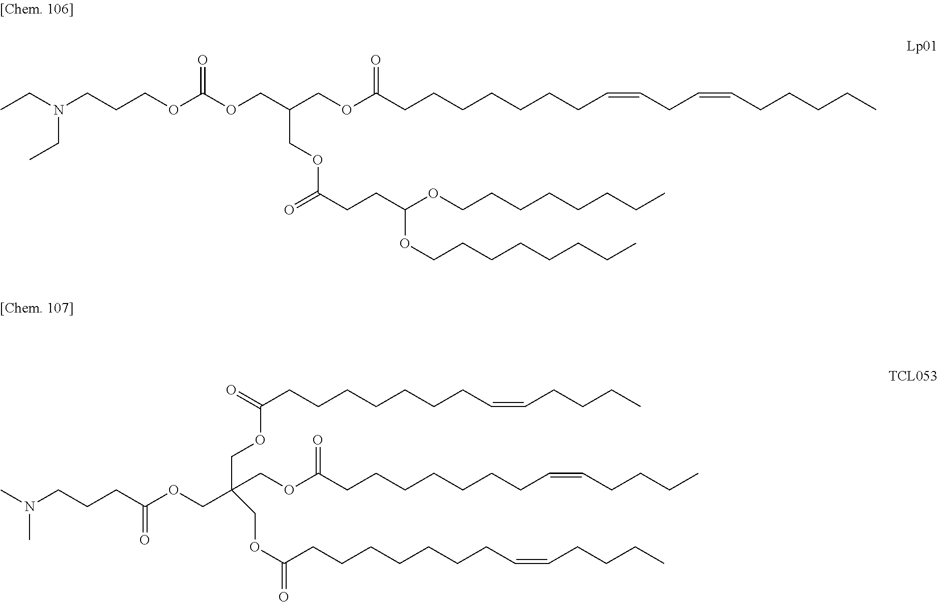

TABLE 6
Physicochemical propeties of DC-cholesterol LNPs with different ionizalbe lipids

| | | Ionizable | | | | |
|---|---|---|---|---|---|---|
| | | lipid:phospho- | | | | Zeta |
| | | lipid:sterol:PEG- | Encapsulation | Z-average | | potential |
| | Ionizable lipid | lipid | (%) | (nm) | pKa | (mV) |
| LNP33 | Lipid A9 | 50:10:38.5:1.5 | 78 | 84.1 | 7.4 | TBA |
| LNP34 | Lipid 29 | 50:10:38.5:1.5 | 87 | 80.3 | 6.7 | TBA |
| LNP35 | ATX-100 | 50:10:38.5:1.5 | 91 | 65.0 | 6.9 | TBA |
| LNP36 | Lp01 | 50:10:38.5:1.5 | 87 | 87.3 | 7.1 | TBA |
| LNP37 | TCL053 | 50:10:38.5:1.5 | 88 | 85.1 | 7.2 | TBA |

Formulations including an siRNA against Cdh5 were intravenously administered to mice at 0.5 mg/kg and organs were harvested 72 hours after injection. After isolation and purification of total RNA from organs, Cdh5 mRNA is quantified relative to a housekeeping gene, Gusb. A PBS control was also tested.

DC-cholesterol substitution will demonstrate silencing of endothelial genes with various ionizable lipids. Cdh5 mRNA expression is measured based on ΔΔCt calculations compared to PBS 3 days post-injection. (0.5 mg/kg siCdh5)

### Example 7: In Vivo Endothelial Cell RNA Delivery with Various Ionizable Lipid

To evaluate whether endothelial RNA delivery by ionizable cholesterol (iChol) LNP is generalizable to various ionizable lipids, we prepared a library of iChol LNPs with different ionizable lipids (Table 7). In this experiment, we selected siRNA against Cdh5 gene, which is expressed specifically in endothelial cells. By using siCdh5, endothelial delivery efficiency can be evaluated without isolating endothelial cells. These LNPs were intravenously administered to mice at 0.5 mg/kg. Subsequently, 48-72 hours post-administration, the respective organs were harvested, and total RNA was extracted utilizing the Quick-RNA MagBead kit (Zymo Research). Following RNA extraction, cDNA was synthesized through reverse transcription, and the gene expression levels of Cdh5 were quantified via real-time PCR.

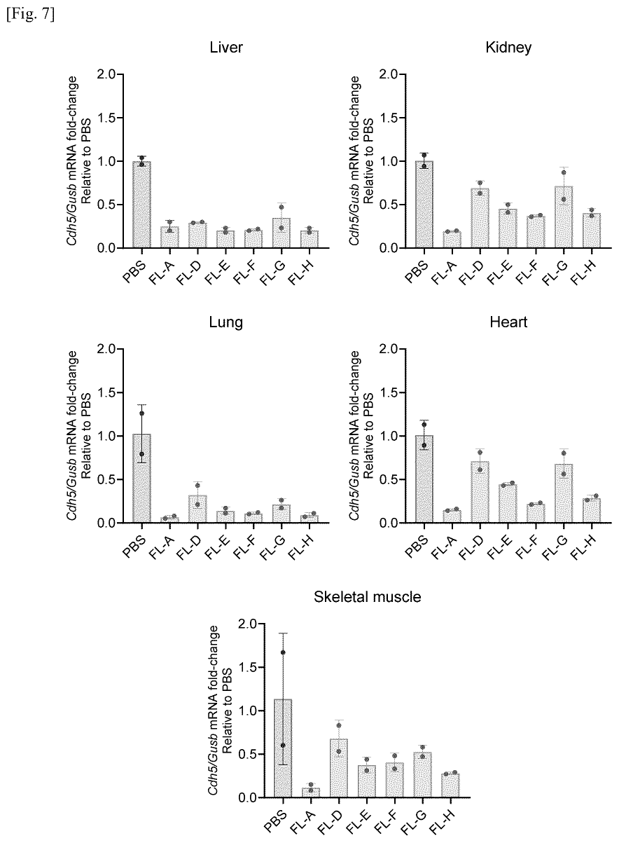

The results are shown in FIG. 7. All the iChol LNP formulations with different ionizable lipid showed significant gene silencing in the liver, kidney, lung, heart and skeletal muscle (quadriceps), demonstrating that iChol LNP formulation is generalizable to various ionizable lipids.

Endothelial Cells: siCdh5

5′-ccAAAAGAGAGAcuGGAuudTsdT-3′
5′-AAUCcAGUCUCUCUUUUGGdTsdT-3′

TABLE 7
Physicochemical propeties of DC-cholesterol LNPs with different ionizalbe lipids

| | | | | | Polymer- | | |
|---|---|---|---|---|---|---|---|
| | | | | | conjugated | | |
| | | IL | PL | Sterol | lipid DMG- | Encapsulation | Z-average |
| LNP ID | IL | FL-A | DSPC | DC-Chol | mPEG2000 | (%) | (nm) |
| LPN38 | FL-A | 50 | 10 | 38.5 | 1.5 | 99 | 98.4 |
| LPN39 | FL-D | 50 | 10 | 38.5 | 1.5 | 96 | 94.6 |
| LPN40 | FL-E | 50 | 10 | 38.5 | 1.5 | 98 | 95.9 |
| LPN41 | FL-F | 50 | 10 | 38.5 | 1.5 | 99 | 106.9 |
| LPN42 | FL-G | 50 | 10 | 38.5 | 1.5 | 97 | 107.2 |
| LPN43 | FL-H | 50 | 10 | 38.5 | 1.5 | 99 | 105.2 |

FIG. 7 shows that iChol LNP formulation is generalizable to various ionizable lipids. The graph shows the mRNA levels of Cdh5 relative to PBS control group. Each symbol represents an individual animal. Data are presented as mean±SD.

### Example 8: In Vivo Endothelial Cell RNA Delivery with Various Ionizable Cholesterol Ratio

### (1) HAPC-Cholesterol

To optimize the ionizable cholesterol ratio, we prepared LNP formulations with different HAPC-cholesterol ratio and test them in vivo. In this example, FL-A was used as the ionizable lipid, and FL-A, DSPC and DMG-mPEG-2000 ratio was fixed as 50%, 10% and 1.5%, respectively (Table 8).

In this experiment, we selected siRNA against Cdh5 gene, which is expressed specifically in endothelial cells. By using siCdh5, endothelial delivery efficiency can be evaluated without isolating endothelial cells. These LNPs were intravenously administered to mice at 0.5 mg/kg. Subsequently, 48-72 hours post-administration, the respective organs were harvested, and total RNA was extracted utilizing the Quick-RNA MagBead kit (Zymo Research). Following RNA extraction, cDNA was synthesized through reverse transcription, and the gene expression levels of Cdh5 were quantified via real-time PCR.

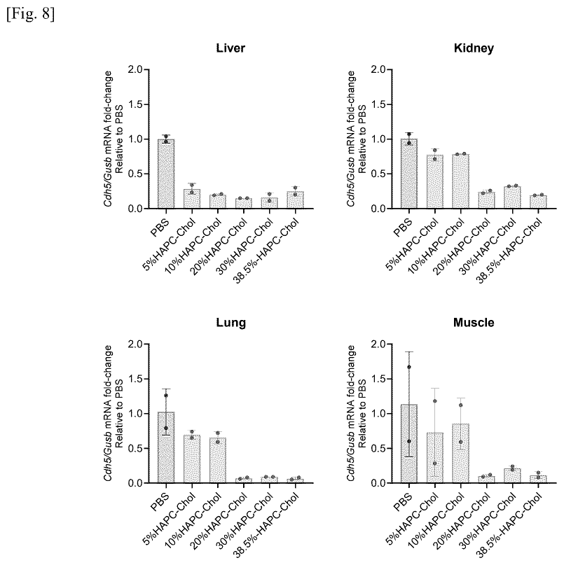

The results are shown in FIG. 8. All the iChol LNPs with different HAPC-cholesterol ratio showed significant gene silencing in the liver, and iChol LNPs with 20% or more HAPC-cholesterol showed more efficient RNA delivery to extrahepatic organs.

Endothelial Cells: siCdh5

5′-ccAAAAGAGAGAcuGGAuudTsdT-3′
5′-AAUCcAGUCUCUCUUUUGGdTsdT-3′

TABLE 8

| | | Total | | | | Sterol | Polymer- | | |
|---|---|---|---|---|---|---|---|---|---|
| | IL/RNA | lipid/ | | | | 2(iChol) | conjugated | | |
| | Mass | RNA Mass | IL | PL | Sterol 1 | HAPC- | lipid DMG- | Encapsulation | Z-average |
| LNP ID | Ratio | Ratio | FL-A | DSPC | Cholesterol | Chol | mPEG2000 | (%) | (nm) |
| LPN44 | 10 | 16.7 | 50 | 10 | 33.5 | 5 | 1.5 | 94 | 114.7 |
| LPN45 | 10 | 17.3 | 50 | 10 | 28.5 | 10 | 1.5 | 96 | 118.5 |
| LPN46 | 10 | 18.0 | 50 | 10 | 18.5 | 20 | 1.5 | 96 | 100.1 |
| LPN47 | 10 | 18.7 | 50 | 10 | 8.5 | 30 | 1.5 | 98 | 95.0 |
| LPN48 | 10 | 19.3 | 50 | 10 | 0 | 38.5 | 1.5 | 99 | 98.4 |

FIG. 8 shows that iChol LNPs deliver RNA to endothelial cells with various HAPC-cholesterol ratio. The graph shows the mRNA levels of Cdh5 relative to PBS control group. Each symbol represents an individual animal. Data are presented as mean±SD.

### Example 9: In Vivo Endothelial Cell RNA Delivery with Various Ionizable Cholesterol Ratio

### (2) DC-Cholesterol

To optimize the ionizable cholesterol ratio, we prepared LNP formulations with different DC-cholesterol ratio and test them in vivo. In this example, FL-A was used as the ionizable lipid, and FL-A, DSPC and DMG-mPEG-2000 ratio was fixed as 50%, 10% and 1.5%, respectively (Table 9).

In this experiment, we selected siRNA against Cdh5 gene, which is expressed specifically in endothelial cells. By using siCdh5, endothelial delivery efficiency can be evaluated without isolating endothelial cells. These LNPs were intravenously administered to mice at 0.5 mg/kg. Subsequently, 48-72 hours post-administration, the respective organs were harvested, and total RNA was extracted utilizing the Quick-RNA MagBead kit (Zymo Research). Following RNA extraction, cDNA was synthesized through reverse transcription, and the gene expression levels of Cdh5 were quantified via real-time PCR.

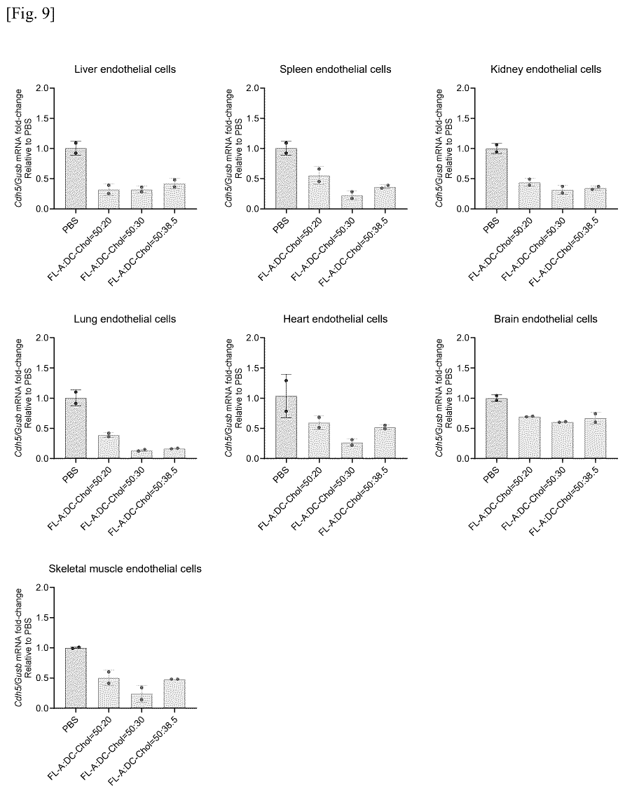

The results are shown in FIG. 9. All the iChol LNPs with different DC-cholesterol ratio showed significant gene silencing in the liver, and iChol LNPs with 20% or more HAPC-cholesterol showed more efficient RNA delivery to extrahepatic organs.

TABLE 9

| | | Total | | | | | Polymer- | | |
|---|---|---|---|---|---|---|---|---|---|
| | IL/RNA | lipid/ | | | | Sterol 2 | conjugated | | |
| | Mass | RNA Mass | IL | PL | Sterol 1 | (iChol) | lipid DMG- | Encapsulation | Z-average |
| LNP ID | Ratio | Ratio | FL-A | DSPC | Cholesterol | DC-Chol | mPEG2000 | (%) | (nm) |
| LPN49 | 10 | 17.4 | 50 | 10 | 18.5 | 20 | 1.5 | 90 | 116.1 |
| LPN50 | 10 | 17.8 | 50 | 10 | 8.5 | 30 | 1.5 | 85 | 96.3 |
| LPN51 | 10 | 18.1 | 50 | 10 | 0 | 38.5 | 1.5 | 88 | 82.9 |

FIG. 9 shows that iChol LNPs deliver RNA to endothelial cells with various DC-cholesterol ratio. The graph shows the mRNA levels of Cdh5 relative to PBS control group. Each symbol represents an individual animal. Data are presented as mean±SD.

### Example 10: In Vivo Endothelial Cell RNA Delivery with Various Ionizable Cholesterol Ratio

### (3) HAPC-Cholesterol

To optimize the ionizable cholesterol ratio, we prepared LNP formulations with different HAPC-cholesterol ratio and test them in vivo. In this example, FL-A was used as the ionizable lipid, and DMG-mPEG-2000 ratio was fixed as 1.5% (Table 10).

In this experiment, we selected siRNA against Cdh5 gene, which is expressed specifically in endothelial cells. By using siCdh5, endothelial delivery efficiency can be evaluated without isolating endothelial cells. These LNPs were intravenously administered to mice at 0.5 mg/kg. Subsequently, 48-72 hours post-administration, the respective organs were harvested, and total RNA was extracted utilizing the Quick-RNA MagBead kit (Zymo Research). Following RNA extraction, cDNA was synthesized through reverse transcription, and the gene expression levels of Cdh5 were quantified via real-time PCR.

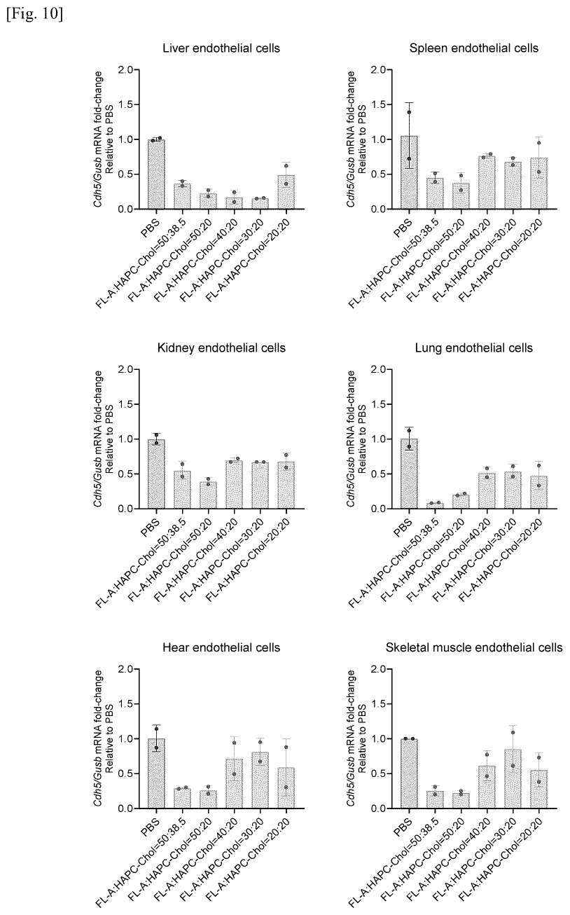

The results are shown in FIG. 10. All the iChol LNPs with different DC-cholesterol ratio showed significant gene silencing in the liver, and iChol LNPs with 20% or more HAPC-cholesterol and 20% or more ionizable lipid showed more efficient RNA delivery to extrahepatic organs.

TABLE 10

| | | Total | | | | | Polymer- | | |
|---|---|---|---|---|---|---|---|---|---|
| | IL/R NA | lipid/ | | | | Sterol 2 | conjugated | | |
| | Mass | RNA Mass | IL | PL | Sterol 1 | HAPC- | lipid DMG- | Encapsulation | Z-average |
| LNP ID | Ratio | Ratio | FL-A | DSPC | Cholesterol | Chol | mPEG2000 | (%) | (nm) |
| LPN52 | 10 | 19.3 | 50 | 10 | 0 | 38.5 | 1.5 | 99 | 98.7 |
| LPN53 | 10 | 18.0 | 50 | 10 | 18.5 | 20 | 1.5 | 96 | 99.6 |
| LPN54 | 8 | 18.0 | 40 | 20 | 18.5 | 20 | 1.5 | 94 | 109.8 |
| LPN55 | 6 | 18.0 | 30 | 30 | 18.5 | 20 | 1.5 | 86 | 135.5 |
| LPN56 | 4 | 18.0 | 20 | 40 | 18.5 | 20 | 1.5 | 90 | 128.9 |

FIG. 10 shows that iChol LNPs deliver RNA to endothelial cells various HAPC-cholesterol ratio. The graph shows the mRNA levels of Cdh5 gene relative to PBS control group. Each symbol represents an individual animal. Data are presented as mean±SD.

### Example 11: Serum Protein-Independent RNA Delivery to Endothelial Cells

Apolipoprotein E (ApoE) is a protein that is found in the bloodstream and is involved in the transport of lipids and cholesterol throughout the body. Recent research has shown that ApoE plays an important role in the uptake and distribution of hepatocyte LNPs. Specifically, it has been found that LNPs are taken up by cells in the liver through a process that is mediated by ApoE which adsorb on LNP surface. In terms of LNPs containing constitutively cationic lipid, it has been reported that cellular uptake mechanism is also serum protein-dependent. Among serum proteins, vitronectin is reported to play a vital role in cellular uptake of these cationic LNPs.
WO2020051220
WO2020051223
Dilliard S. A. Proc. Nat. Assoc. Sci. 2022. https://doi.org/10.1073/pnas.2109256118
Dilliard S. A. J. Cont. Rel. 2023. https://doi.org/10.1016/j.jconrel.2023.07.058.

To analyze the serum protein-dependence of ionizable cholesterol LNP uptake, in vitro transfection efficiency was evaluated in serum-containing and serum-free culture media. LNP formulations encapsulating siCdh5 were prepared as described in materials and methods (Table 11), and the resulting LNPs were further tested in bEND.3 murine brain endothelial cell line. bEND.3 cells were cultured using the standard culture condition and transferred to 96-well plates at a density of 10,000-15,000 cells per well. bEND.3 endothelial cells were transfected at a concentration of 100 nM of siCdh5 per well. After incubating for 2 hours, cell culture media containing LNP was removed, and cells were washed with serum-free media, and then cultured in serum-containing media. After overnight incubation total RNA was extracted by QuickExtract RNA Extraction Kit (LGC Biosearch technologies), and cDNA was synthesized by reverse transcription. Cdh5 expression level was quantified by real time PCR.

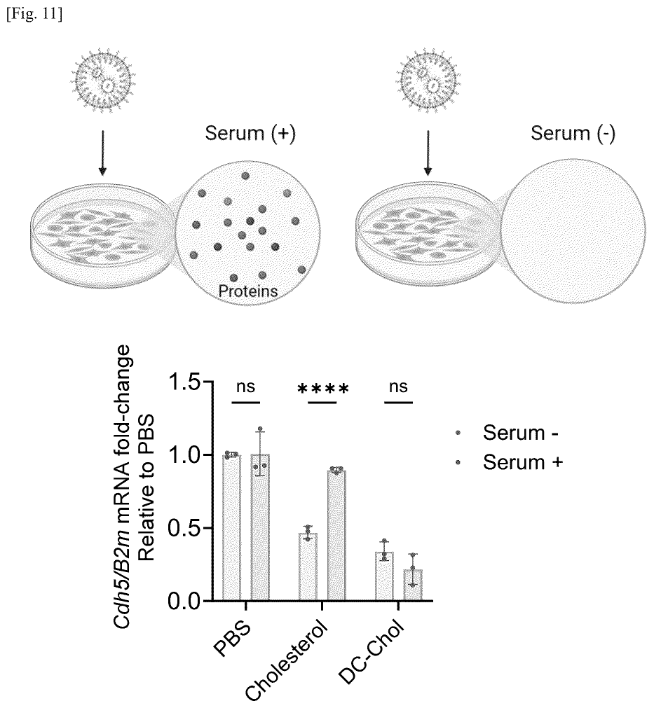

The results are shown in FIG. 11. While hepatocyte LNP showed more efficient gene silencing in serum-containing media, ionizable cholesterol LNP showed similar gene silencing efficiency in serum-free and serum-containing media, suggesting that ionizable cholesterol LNP uptake is independent of serum proteins.

TABLE 11

| | | Total | | | | | Polymer- |
|---|---|---|---|---|---|---|---|
| | IL/RNA | lipid/ | | | | | conjugated |
| | Mass | RNA Mass | | IL | PL | | lipid DMG- |
| LNP ID | Ratio | Ratio | Sterol | FL-A | DSPC | Sterol | mPEG2000 |
| Comparative | 10 | 16.7 | Cholesterol | 50 | 10 | 38.5 | 1.5 |
| example | | | | | | | |
| (hepatocyte LNP) | | | | | | | |
| LPN57 | | | | | | | |
| LPN58 | 10 | 19.3 | DC-Chol | 50 | 10 | 38.5 | 1.5 |

FIG. 11 shows that Endothelial cells take up iChol LNPs via serum-independent manner.

Top: Schematic of the in vitro experimental design showing bEND.3 endothelial cells treated with siRNA-LNP in serum-containing and serum-free media Bottom: The graph shows the mRNA levels of Cdh5 relative to PBS control group. Each symbol represents an individual well. Data are presented as mean±SD.

### Example 12: In Vitro Endothelial RNA Delivery with Various LNP Formulations

To optimize the DC-cholesterol ratio, we prepared LNP formulations encapsulating firefly luciferase mRNA (TriLink) with various lipid ratios, and the resulting LNPs were further tested in bEND.3 murine brain endothelial cell line. bEND.3 cells were cultured using the standard culture condition and transferred to 96-well plates at a density of 10,000-15,000 cells per well. Before transfection, cell culture media was replaced with serum-free media. bEND.3 endothelial cells were transfected at a dose of 1 μg per well. A total of 29 LNP formulations, including hepatocyte LNPs, were transfected into bEND.3 cells. After incubating for 2 hours, luciferase expression level was quantified by Steady-Glo™ Luciferase Assay System (Promega).

$$\begin{matrix}Ionizable lipid 10 - 80 < 20 - 70 < 25 - 50 \\ Phospholipid 0 - 40 < 5 - 35 < 10 - 30 \\ Ionizable sterol 5 - 60 < 7.5 - 55 < 10 - 50 \\ Sterol + ionizable sterol 10 - 60 < 20 - 50\end{matrix}$$

TABLE 12
LNP formulation with various lipid ratios (Ionizable lipid/RNA ratio was fixed at 10)

| | | | | | Polymer- | Fold increase of |
|---|---|---|---|---|---|---|
| | | | | Sterol 2 | conjugated | delivery |
| | IL | PL | Sterol 1 | DC- | lipid DMG- | (Relative to |
| LNP ID | FL-F | DSPC | Cholesterol | Cholesterol | mPEG2000 | hepatocyte LNP) |
| PBS | — | — | — | — | — | |
| Comparative | 50 | 10 | 38 | 0 | 2 | 1 |
| example | | | | | | |
| (hepatocyte | | | | | | |
| LNP) | | | | | | |
| LPN59 | | | | | | |
| LPN60 | 20 | 30 | 0 | 48 | 2 | 3 |
| LPN61 | 20 | 30 | 10 | 38 | 2 | 6 |
| LPN62 | 50 | 20 | 8 | 20 | 2 | 6 |
| LPN63 | 20 | 30 | 26 | 22 | 2 | 7 |
| LPN64 | 20 | 30 | 38 | 10 | 2 | 9 |
| LPN65 | 50 | 10 | 13 | 25 | 2 | 9 |
| LPN66 | 40 | 20 | 18 | 20 | 2 | 10 |
| LPN67 | 40 | 10 | 38 | 10 | 2 | 11 |
| LPN68 | 43 | 25 | 10 | 20 | 2 | 12 |
| LPN69 | 26 | 24 | 8 | 40 | 2 | 13 |
| LPN70 | 50 | 24 | 14 | 10 | 2 | 14 |
| LPN71 | 50 | 16 | 0 | 32 | 2 | 19 |
| LPN72 | 40 | 10 | 28 | 20 | 2 | 20 |
| LPN73 | 40 | 10 | 10 | 38 | 2 | 22 |
| LPN74 | 50 | 16 | 22 | 10 | 2 | 27 |
| LPN75 | 50 | 10 | 0 | 38 | 2 | 28 |
| LPN76 | 50 | 24 | 0 | 24 | 2 | 33 |
| LPN77 | 28 | 30 | 15 | 25 | 2 | 37 |
| LPN78 | 32 | 18 | 38 | 10 | 2 | 38 |
| LPN79 | 38 | 20 | 30 | 10 | 2 | 43 |
| LPN80 | 40 | 10 | 0 | 48 | 2 | 44 |
| LPN81 | 43 | 15 | 14 | 26 | 2 | 44 |
| LPN82 | 28 | 30 | 0 | 40 | 2 | 45 |
| LPN83 | 50 | 28 | 0 | 20 | 2 | 53 |
| LPN84 | 40 | 30 | 0 | 28 | 2 | 64 |
| LPN85 | 32 | 16 | 0 | 50 | 2 | 67 |
| LPN86 | 50 | 10 | 28 | 10 | 2 | 97 |
| LPN87 | 37 | 22 | 0 | 39 | 2 | 106 |

### Example 13: LNP-Mediated Endothelial Cell Damage Analysis In Vitro

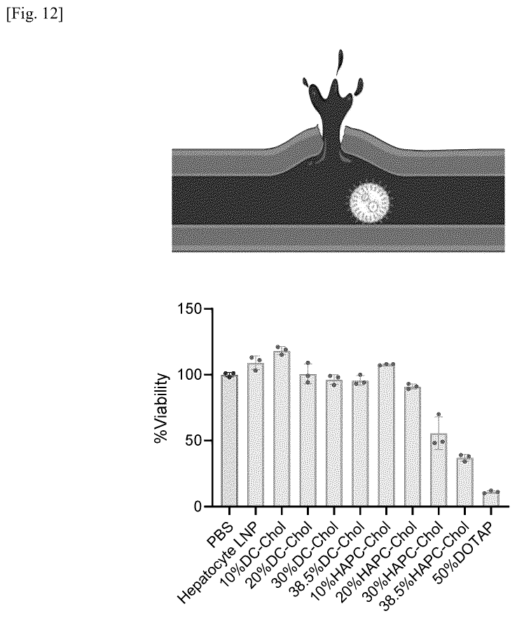

In vivo endothelial cell damage potentially causes internal hemorrhage (FIG. 12). To quantify the cytotoxicity of the LNPs on endothelial cell, a murine endothelial cell line, bEND.3, was used. 250 nM of siRNA against Cdh5 encapsulated in LNPs was administered to 10,000 cells in a 96-well plate, and these cells were incubated for 24 h post transfection prior to analysis where cell viability was determined using CCK-8 assay kit (Dojindo) according to the manufacturer's protocol and normalized to PBS treatment group.

The results are shown in FIG. 12. These results demonstrated that LNPs containing ionizable cholesterol is more tolerable than LNPs containing DOTAP, a constitutively cationic lipid.

TABLE 13

| | | | | | | Polymer- | |
|---|---|---|---|---|---|---|---|
| | | | | | | conjugated | |
| | | IL | PL | Sterol 1 | | lipid DMG- | % Viability |
| LNP ID | Sterol 2 | FL-A | DSPC | Cholesterol | Sterol 2 | mPEG2000 | (vs. PBS) |
| Comparative | None | 50 | 10 | 38.5 | 0 | 1.5 | 109 |
| example | | | | | | | |
| (hepatocyte LNP) | | | | | | | |
| LPN88 | | | | | | | |
| LPN89 | DC-Chol | 50 | 10 | 28.5 | 10 | 1.5 | 118 |
| LPN90 | DC-Chol | 50 | 10 | 18.5 | 20 | 1.5 | 100 |
| LPN91 | DC-Chol | 50 | 10 | 8.5 | 30 | 1.5 | 96 |
| LPN92 | DC-Chol | 50 | 10 | 0 | 38.5 | 1.5 | 96 |
| LPN93 | HAPC- | 50 | 10 | 28.5 | 10 | 1.5 | 108 |
| | Chol | | | | | | |
| LPN94 | HAPC- | 50 | 10 | 18.5 | 20 | 1.5 | 91 |
| | Chol | | | | | | |
| LPN95 | HAPC- | 50 | 10 | 8.5 | 30 | 1.5 | 56 |
| | Chol | | | | | | |
| LPN96 | HAPC- | 50 | 10 | 0 | 38.5 | 1.5 | 37 |
| | Chol | | | | | | |
| Comparative | (DOTAP) | 25 | 5 | 19 | (50% | 1.5 | 11 |
| example | | | | | DOTAP) | | |

Ionizable lipid/RNA ratio was fixed at 10

FIG. 12 shows that iChol LNP has lower endothelial cellular toxicity than cationic lipid containing LNPs
Top: Schematic representation of the potential internal hemorrhage induced by LNP-mediated endothelial cell damage
Bottom: The graph shows the cellular viability relative to the PBS control group. Each symbol represents an individual well. Data are presented as mean±SD.

### Example 14: In Vitro Hemocompatibility Analysis Using Human Primary Red Blood Cells

To evaluate hemocompatibility of LNPs, hemolysis assay was conducted. In the hemolysis assay, human primary red blood cells (RBCs, Innovative Research) were washed with PBS three times, and 90 μL of 4% vol/vol RBCs suspension in PBS were transferred to 96-well plate. Subsequently, 10 μL of siRNA LNP solution containing 150 μg/mL of total lipid was added to RBCs suspension. Following one hour incubation at 37° C., the plate was centrifuged at 1,000×g for 5 min, and 80 μL of supernatant was transferred to clear flat bottom 96-well plate. Hemoglobin release was quantified by measuring UV-vis absorbance at 490 nm. A cationic detergent, Triton X-100, which completely dissolves RBC membranes, was used as a positive control, and hemoglobin release was normalized to Triton X-100 treated group.

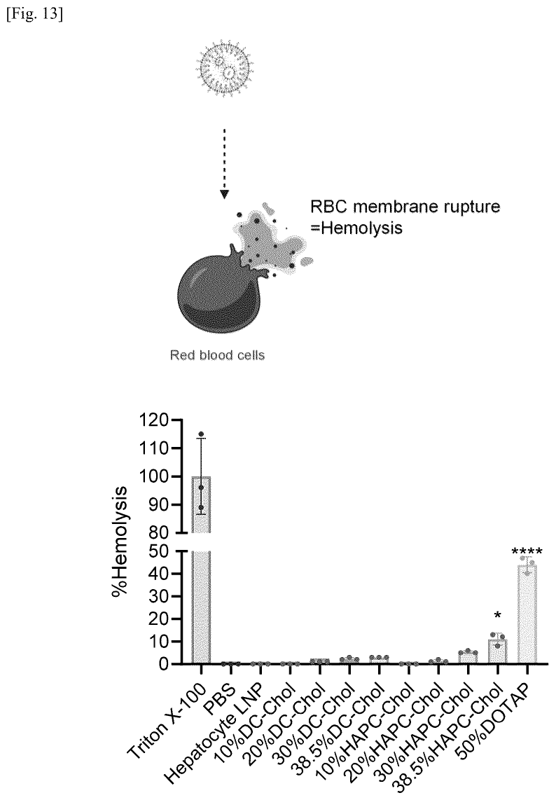

The results are shown in FIG. 13. 50% DOTAP LNP showed significant hemolysis while LNP with ionizable cholesterol such as DC-Cholesterol and HAPC-Cholesterol didn't. These data demonstrate the higher hemocompatibility of ionizable cholesterol LNPs.

TABLE 14

| | | | | | | Polymer- | % Hemolys |
|---|---|---|---|---|---|---|---|
| | | | | | | conjugated | is (Normalized |
| | | IL | PL | Sterol 1 | | lipid DMG- | to Triton |
| LNP ID | Sterol 2 | FL-A | DSPC | Cholesterol | Sterol 2 | mPEG2000 | X-100) |
| Comparative | None | 50 | 10 | 38.5 | 0 | 1.5 | 0 |
| example | | | | | | | |
| (hepatocyte LNP) | | | | | | | |
| LPN97 | | | | | | | |
| LPN98 | DC-Chol | 50 | 10 | 28.5 | 10 | 1.5 | 0 |
| LPN99 | DC-Chol | 50 | 10 | 18.5 | 20 | 1.5 | 1 |
| LPN100 | DC-Chol | 50 | 10 | 8.5 | 30 | 1.5 | 2 |
| LPN101 | DC-Chol | 50 | 10 | 0 | 38.5 | 1.5 | 3 |
| LPN102 | HAPC- | 50 | 10 | 28.5 | 10 | 1.5 | 0 |
| | Chol | | | | | | |
| LPN103 | HAPC- | 50 | 10 | 18.5 | 20 | 1.5 | 1 |
| | Chol | | | | | | |
| LPN104 | HAPC- | 50 | 10 | 8.5 | 30 | 1.5 | 5 |
| | Chol | | | | | | |
| LPN105 | HAPC- | 50 | 10 | 0 | 38.5 | 1.5 | 11 |
| | Chol | | | | | | |
| Comparative | (DOTAP) | 25 | 5 | 19 | (50) | 1.5 | 44 |
| example | | | | | | | |
| LPN106 | | | | | | | |

Ionizable lipid/RNA ratio was fixed at 10

FIG. 13 shows that iChol LNP has lower hemolysis activity than cationic lipid containing LNPs
Top: Schematic representation of the hemolysis assay
Bottom: The graph shows the % homolysis normalized to Triton X-100 positive control group. Each symbol represents an individual well. Data are presented as mean±SD.
Statistically significant differences were evaluated using one-way ANOVA and Dunnett's post test. Adjusted P values of less than 0.05 were considered statistically significant with *p<0.05; **p<0.01; ***p<0.001; and ****p<0.0001.

### Example 15: In Vivo Hepatic Stellate Cell Delivery

To analyze the specific cell types within the liver that underwent transfection by ionizable cholesterol LNPs, we conducted a meticulous assessment of gene silencing efficiency within individual cell populations. To obtain cell-type specific information, we employed distinct siRNAs targeting genes characterized by cell-type specific expression patterns. We prepared LNPs encapsulating the following siRNAs (Table 15), and these LNPs were intravenously administered to mice at. Subsequently, 48-72 hours post-administration, the respective organs were harvested, and total RNA was extracted utilizing the Quick-RNA MagBead kit (Zymo Research). Following RNA extraction, cDNA was synthesized through reverse transcription, and the gene expression levels of Cdh5, Reln and Sirpa were quantified via real-time PCR. For Fvii gene silencing, serum Factor VII protein level was quantified by using BIOPHEN™ FVII assay kit (Aniara).

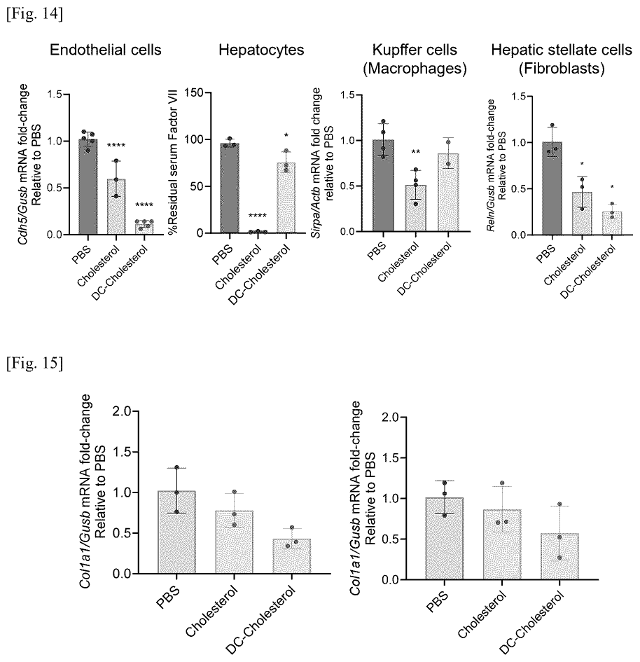

The results are shown in FIG. 14. While hepatocyte LNP showed gene silencing in all the cell types evaluated in the liver, ionizable cholesterol LNP showed more efficient and specific gene silencing in endothelial cells and hepatic stellate cells.

Hepatocyte: siFvii
5′-GGAUfCfAUfCfUfCfAAGUfCfUfUfACfdTsdT-3′
5′-GUfAAGACfUfUfGAGAUfGAUfCfCfdTsdT-3′
Endothelial cells: siCdh5
5′-ccAAAAGAGAGAcuGGAuudTsdT-3′
5′-AAUCcAGUCUCUCUUUUGGdTsdT-3′
Hepatic stellate cells: siReln
5′-GGmUmCmUmCAAGmCmCAmCmUmCGmUmUmUdTsdT-3′
5′-AAACGAGUGGCUUGAGACCdTsdT-3′
Kupffer cells: si Sirpa
5′-mCmUAAmCAAmCmCAmCAmCAGAAmUAdTsdT-3′
5′-mUAUUCUGUGUGGUUGUmUAGdTsdT-3′

TABLE 15

| | | | | | | | | Polymer- |
|---|---|---|---|---|---|---|---|---|
| | IL/RNA | Total | | | | | | conjugated |
| | Mass | lipid/ | | | IL | PL | | lipid DMG- |
| LNP ID | Ratio | RNA Mass | siRNA | Sterol | FL-A | DSPC | Sterol | mPEG2000 |
| Comparative | 10 | 16.7 | siCdh5 | Cholesterol | 50 | 10 | 38.5 | 1.5 |
| example | | | | | | | | |
| (hepatocyte | | | | | | | | |
| LNP) | | | | | | | | |
| LPN107 | | | | | | | | |
| LPN108 | 10 | 19.3 | siCdh5 | DC-Chol | 50 | 10 | 38.5 | 1.5 |
| Comparative | 10 | 16.7 | siFvii | Cholesterol | 50 | 10 | 38.5 | 1.5 |
| example | | | | | | | | |
| (hepatocyte | | | | | | | | |
| LNP) | | | | | | | | |
| LPN109 | | | | | | | | |
| LPN110 | 10 | 19.3 | siFvii | DC-Chol | 50 | 10 | 38.5 | 1.5 |
| Comparative | 10 | 16.7 | siSirpa | Cholesterol | 50 | 10 | 38.5 | 1.5 |
| example | | | | | | | | |
| (hepatocyte | | | | | | | | |
| LNP) | | | | | | | | |
| LPN111 | | | | | | | | |
| LPN112 | 10 | 19.3 | siSirpa | DC-Chol | 50 | 10 | 38.5 | 1.5 |
| Comparative | 10 | 16.7 | siReln | Cholesterol | 50 | 10 | 38.5 | 1.5 |
| example | | | | | | | | |
| (hepatocyte | | | | | | | | |
| LNP) | | | | | | | | |
| LPN113 | | | | | | | | |
| LPN114 | 10 | 19.3 | siReln | DC-Chol | 50 | 10 | 38.5 | 1.5 |

FIG. 14 shows that iChol LNP deliver RNA to liver endothelial cells and hepatic stellate cells. The graphs show the mRNA levels of Cdh5 gene (endothelial cells), Sirpa gene (Kupffer cells), or Reln gene (Hepatic stellate cells), or serum Factor VII protein levels (hepatocytes) relative to PBS control group. siRNA-LNPs were intravenously administered to mice at a dose of siCdh5 0.3 mg/kg, siFvii 0.3 mg/kg, siSirpa 0.4 mg/kg, and siReln 0.2 mg/kg, respectively. Each symbol represents an individual animal. Data are presented as mean±SD. Statistically significant differences were evaluated using one-way ANOVA and Dunnett's post test. Adjusted P values of less than 0.05 were considered statistically significant with *p<0.05; **p<0.01; ***p<0.001; and ****p<0.0001.

### Example 16: In Vivo RNA Delivery to Extrahepatic Extracellular Matrix Producing Cells

Since stellate cells have been also found at extrahepatic organs such as the pancreas, lung, kidney, intestine, spleen, adrenal gland, ductus deferens and vocal cords, we also evaluated RNA delivery to extrahepatic stellate cells using siRNA against Col1a1, which is expressed specifically in extracellular matrix producing cells such as stellate cells and fibroblast. We prepared LNPs encapsulating the siCol1a1 (Table 16), and these LNPs were intravenously administered to mice at a dose of 0.5 mg/kg siCola1a. Subsequently, 48-72 hours post-administration, the respective organs were harvested, and total RNA was extracted utilizing the Quick-RNA MagBead kit (Zymo Research). Following RNA extraction, cDNA was synthesized through reverse transcription, and the Col1a1 expression levels were quantified via real-time PCR.

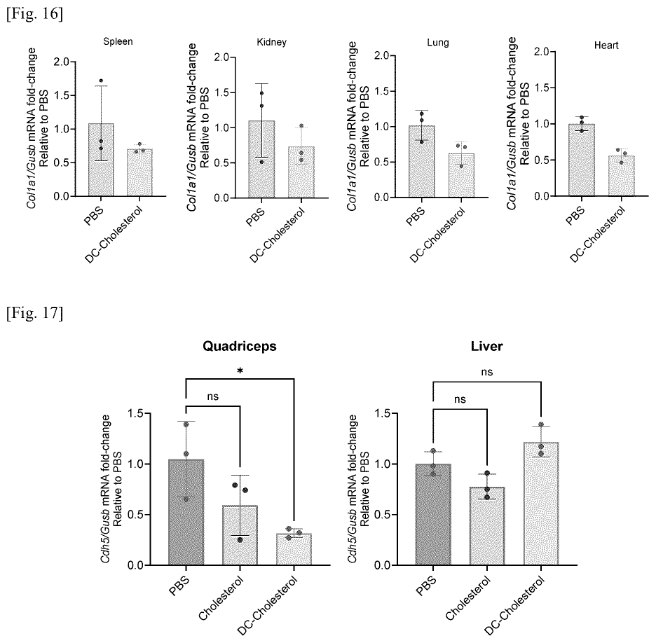

The results are shown in FIG. 15 and FIG. 16. Compared to hepatocyte LNP, DC-cholesterol LNP showed more efficacious gene silencing in pancreatic and colonic stellate cells. Also, gene silencing in extracellular matrix producing cells at other organs was also confirmed.

siCol1a1
5′-GmUmCmUAGAmCAmUGmUmUmCAGmCmUmUdTsdT-3′
5′-AAGCUGAAmCAUGUCmUAGACdTsdT-3′

TABLE 16

| | | Total | | | | | | Polymer- |
|---|---|---|---|---|---|---|---|---|
| | IL/R | lipid/ | | | | | | conjugated |
| | NA Mass | RNA Mass | | | IL | PL | | lipid DMG- |
| LNP ID | Ratio | Ratio | siRNA | Sterol | FL-A | DSPC | Sterol | mPEG2000 |
| Comparative | 10 | 16.7 | siCol1a1 | Cholesterol | 50 | 10 | 38.5 | 1.5 |
| example | | | | | | | | |
| (hepatocyte | | | | | | | | |
| LNP) | | | | | | | | |
| LPN115 | | | | | | | | |
| LPN116 | 10 | 19.3 | siCol1a1 | DC-Chol | 50 | 10 | 38.5 | 1.5 |

FIG. 15 shows that iChol LNPs deliver RNA to extrahepatic stellate cells. The graphs show the mRNA levels of Col1a1 gene in the pancreas (left) and colon (right) relative to PBS control group, respectively. Each symbol represents an individual animal. Data are presented as mean±SD.

FIG. 16 shows that iChol LNPs deliver RNA to extrahepatic extracellular matrix producing cells. The graphs show the mRNA levels of Col1a1 gene in the spleen, kidney, lung and pancreas relative to PBS control group, respectively. Each symbol represents an individual animal. Data are presented as mean±SD.

### Example 17: Intramuscular Administration and Delivery to Myocytes and Endothelial Cells

Lipid Nanoparticles (LNPs) have proven to be highly versatile delivery vehicles beyond their well-known role in vaccine development. When considering intramuscular administration, LNPs offer several potential advantages in various therapeutic areas by localizing therapeutic effect in the affected muscle groups, reducing the potential for systemic side effects and improving treatment specificity.

To evaluate whether LNP effect is localized, we administered iChol LNP intramuscularly to quadriceps and quantified gene silencing in the quadriceps (injection side) and the liver since hepatocyte LNP is reported to deliver significant amount of RNA to the liver when administered intramuscularly. We also assessed gene silencing in skeletal myocyte in the quadriceps (injection side). To obtain cell-type specific information, we employed distinct siRNAs targeting genes characterized by cell-type specific expression patterns. We prepared LNPs encapsulating the following siRNAs (Table 17), and these LNPs were intramuscularly administered to mice at a dose of 0.2 mg/kg siMstn. Subsequently, 48-72 hours post-administration, the quadriceps and the liver were harvested, and total RNA was extracted utilizing the Quick-RNA MagBead kit (Zymo Research). Following RNA extraction, cDNA was synthesized through reverse transcription, and the gene expression levels of Cdh5 and Mstn were quantified via real-time PCR.

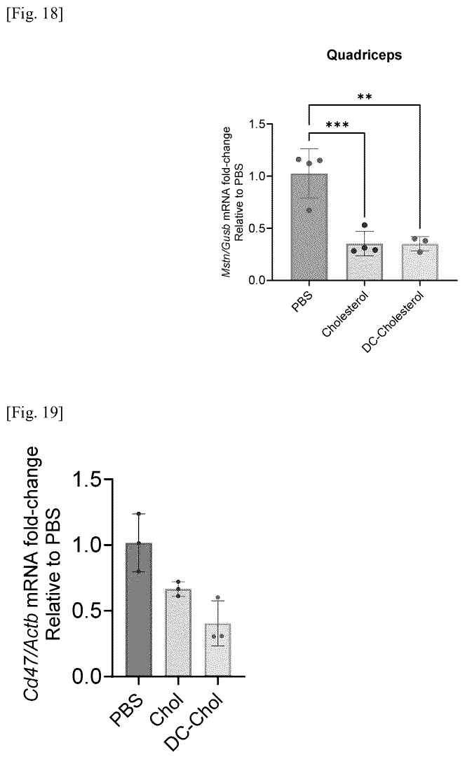

The results are shown in FIG. 17 and FIG. 18. DC-cholesterol LNP showed more potent gene silencing in muscular endothelial cells while it didn't show off-target liver endothelial cell delivery. As for myocytes, hepatocyte LNP and DC-Chol LNP showed comparable gene silencing. This low off-target delivery property in local administration may be potentially advantageous when applied to local tissue regeneration therapy.

Endothelial cells: siCdh5
5′-ccAAAAGAGAGAcuGGAuudTsdT-3′
5′-AAUCcAGUCUCUCUUUUGGdTsdT-3′
Myocytes: siMstn
5′-AmUGGmCAAAGAAmCAAAmUAAmUdTsdT-3′
5′-AUmUAUUUGUUCUUUGCmCAUdTsdT-3′

TABLE 17

| | | Total | | | | | | Polymer- |
|---|---|---|---|---|---|---|---|---|
| | IL/R | lipid/ | | | | | | conjugated |
| | NA Mass | RNA Mass | | | IL | PL | | lipid DMG- |
| LNP ID | Ratio | Ratio | siRNA | Sterol | FL-A | DSPC | Sterol | mPEG2000 |
| Comparative | 10 | 16.7 | siCdh5 | Cholesterol | 50 | 10 | 38.5 | 1.5 |
| example | | | | | | | | |
| (hepatocyte | | | | | | | | |
| LNP) | | | | | | | | |
| LPN117 | | | | | | | | |
| LPN118 | 10 | 19.3 | siCdh5 | DC-Chol | 50 | 10 | 38.5 | 1.5 |
| Comparative | 10 | 16.7 | siMstn | Cholesterol | 50 | 10 | 38.5 | 1.5 |
| example | | | | | | | | |
| (hepatocyte | | | | | | | | |
| LNP) | | | | | | | | |
| LPN119 | | | | | | | | |
| LPN120 | 10 | 19.3 | siMstn | DC-Chol | 50 | 10 | 38.5 | 1.5 |

FIG. 17 shows that Intramuscularly administered ionizable cholesterol LNP deliver RNA to endothelial cells in skeletal muscle around the injected site with minimized off-target delivery to liver endothelial cells
(Left) liver endothelial cell (Right) endothelial cells in muscle at the injection site. The graphs show the mRNA levels of Cdh5 in the liver (left) and quadriceps (right) relative to PBS control group. Each symbol represents an individual animal. Data are presented as mean±SD. Statistically significant differences were evaluated using one-way ANOVA and Dunnett's post test. Adjusted P values of less than 0.05 were considered statistically significant with *p<0.05; **p<0.01; ***p<0.001; and ****p<0.0001.

FIG. 18 shows that intramuscularly administered LNP deliver RNA to skeletal myocyte around the injected site. The graph shows the mRNA levels of Mstn in quadriceps relative to PBS control group. Each symbol represents an individual animal. Data are presented as mean±SD. Statistically significant differences were evaluated using one-way ANOVA and Dunnett's post test. Adjusted P values of less than 0.05 were considered statistically significant with *p<0.05; **p<0.01; ***p<0.001; and ****p<0.0001.

### Example 18: In Vitro Cancer Cell Delivery

To test the applicability to cancer cells, ionizable cholesterol LNPs were tested in cancer model mice. We used B16F10 murine melanoma lung metastasis model where the cancer cells were intravenously administered.

We prepared LNPs encapsulating an siRNA against Cd47 gene encoding CD47 protein (Table 18). CD47 is a ubiquitous membrane receptor, and the interaction between CD47 and signal regulatory protein α (SIRPα), which is expressed on the macrophages, transduces an inhibitory signal that suppresses the phagocytic activity of macrophages. CD47 expression levels are reported to be elevated in various cancerous cells, resulting in the escape from immunosurveillance by innate immune systems and tumor progression.

In vitro transfection efficiency was evaluated in serum-free culture media. LNP formulations encapsulating siCD47 were prepared as described in materials and methods, and the resulting LNPs were further tested in B16F10 murine melanoma cell line. B16F10 cells were cultured using the standard culture condition and transferred to 96-well plates at a density of 10,000-15,000 cells per well. B16F10 cells were transfected at a concentration of 30 nM of siCD47 per well. After incubating for 2 hours, cell culture media containing LNP was removed, and cells were washed with serum-free media, and then cultured in serum-containing media. After overnight incubation total RNA was extracted by QuickExtract RNA Extraction Kit (LGC Biosearch technologies), and cDNA was synthesized by reverse transcription. Cd47 expression level was quantified by real time PCR.

The results are shown in FIG. 19. Ionizable cholesterol LNP showed more efficient gene silencing than hepatocyte LNP.

siCd47
5′-mCmCGAAGAAAmUGmUmUmUGmUGAAdTsdT-3′
5′-UUmCAmCAAAmCAUUUCUUCGGdTsdT-3′

TABLE 18

| | | Total | | | | | Polymer- |
|---|---|---|---|---|---|---|---|
| | IL/R | lipid/ | | | | | conjugated |
| | NA Mass | RNA Mass | | IL | PL | | lipid DMG- |
| LNP ID | Ratio | Ratio | Sterol | FL-A | DSPC | Sterol | mPEG2000 |
| Comparative | 10 | 16.7 | Cholesterol | 50 | 10 | 38.5 | 1.5 |
| example | | | | | | | |
| (hepatocyte LNP) | | | | | | | |
| LPN121 | | | | | | | |
| LPN122 | 10 | 19.3 | DC-Chol | 50 | 10 | 38.5 | 1.5 |

1. A method for delivering a therapeutic agent to endothelial cells, mesenchymal cells, or cancer cells, which comprises administering a lipid composition to a subject,
wherein the lipid composition comprises the therapeutic agent and lipid nanoparticle,
and wherein the lipid nanoparticle comprises an ionizable lipid and a compound represented by formula (1) or a salt thereof. 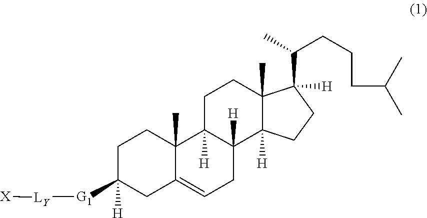
wherein G₁ represents —C(O)—, —OC(O)—, —O(CO)O— or —C(O)O—,
L_{Y} represents a single bond, an alkylene group having 1-14 carbon atoms, a substituted alkylene group having 1-14 carbon atoms, a heteroalkylene group having 1-14 carbon atoms, and a substituted heteroalkylene group having 1-14 carbon atoms,
X represents a basic functional group.

2. The method of claim 1, wherein the basic functional group represented by X is an amino group, a substituted amino group, a guanidino group, a 5 or 6 membered ring heterocyclic alkyl group, or a 5 or 6 membered ring heterocyclic aryl group.

3. The method according to claim 1, wherein the compound represented by formula (1) is a compound represented by formula (2) 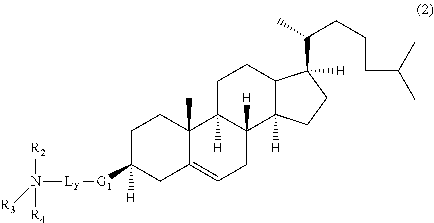
wherein G₁ represents —C(O)—, —OC(O)—, —O(CO)O— or —C(O)O—,
L_{Y} represents a single bond, an alkylene group having 1-14 carbon atoms, a substituted alkylene group having 1-14 carbon atoms, a heteroalkylene group having 1-14 carbon atoms, and a substituted heteroalkylene group having 1-14 carbon atoms,
R², R³ and R⁴ each independently represents a hydrogen atom, a hydrocarbon group having 1 to 4 carbon atoms which may be substituted with a hydroxyl group, or —C(NH₂)=NH₂ and one of R², and
R³ and R⁴ may be absent.

4. The method of claim 3, wherein the compound represented by formula (2) is a compound represented by formula (3) 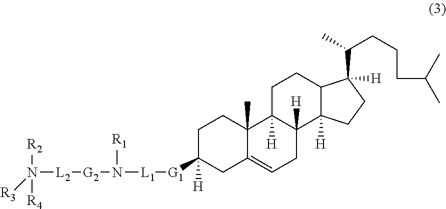
wherein G₁ represents —C(O)—, —OC(O)—, —O(CO)O— or —C(O)O—,
L₁ represents a single bond or an alkylene group having 1 to 6 carbon atoms,
R₁ represents a hydrogen atom, a hydrocarbon group containing 1 to 4 carbon atoms, or an aminoalkyl group having 1 to 4 carbon atoms,
G₂ represents a single bond, —C(O)—, —OC(O)—, —O(CO)O— or —C(O)O—,
L₂ represents an alkylene group having 1 to 6 carbons which may have an amino group,
R², R³ and R⁴ each independently represent a hydrogen atom, a hydrocarbon group having 1 to 4 carbon atoms which may be substituted with a hydroxyl group, or —C(NH₂)=NH₂, and one of R², R³ and R⁴ may be absent.

5. The method of claim 4, wherein the compound represented by formula (3) is a compound represented by formula (3A): 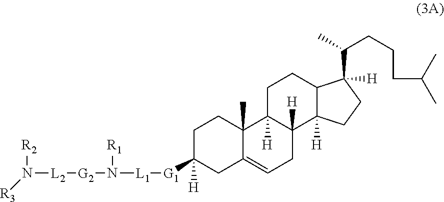
wherein G₁ represents —C(O)—, —OC(O)—, —O(CO)O— or —C(O)O—,
L₁ represents a single bond or an alkylene group having 1 to 6 carbon atoms,
R₁ represents a hydrogen atom, a hydrocarbon group having 1 to 4 carbon atoms, or an aminoalkyl group having 1 to 4 carbon atoms,
G₂ represents a single bond, —C(O)—, —OC(O)—, —O(CO)O— or —C(O)O—,
L₂ represents an alkylene group having 1 to 6 carbons which may have an amino group, and
R² and R³ is each independently a hydrogen atom, a hydrocarbon group having 1 to 4 carbon atoms which may be substituted with a hydroxyl group, or —C(NH₂)=NH₂.

6. The method according to claim 1, wherein the compound represented by formula (1) or a salt thereof is any of the following:

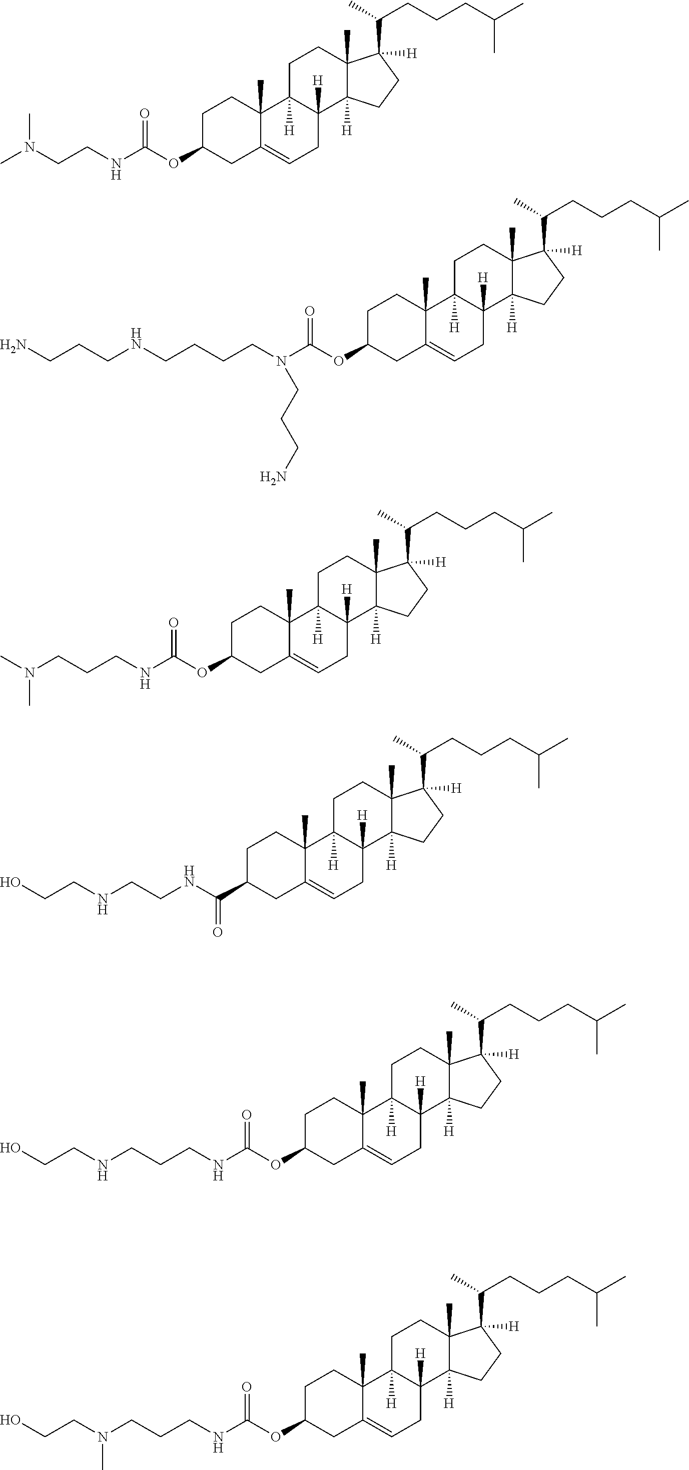

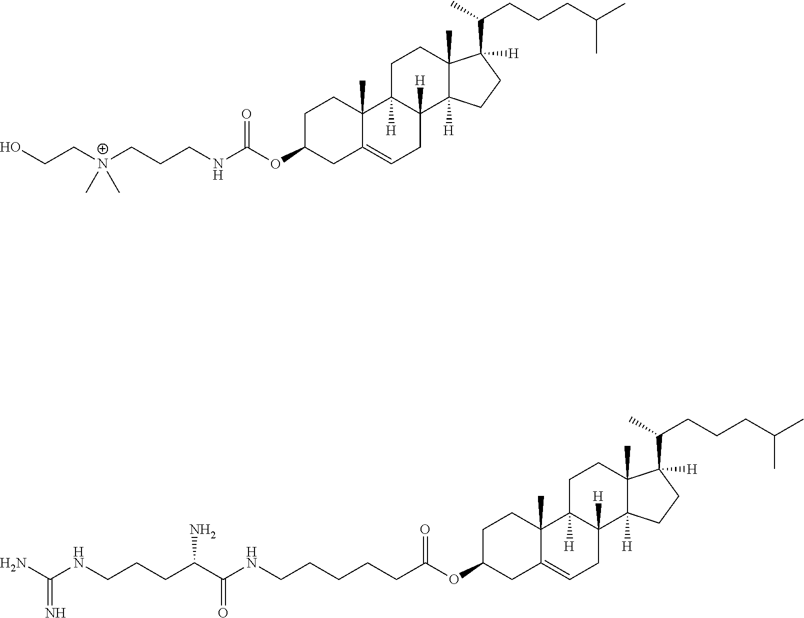

7. The method of claim 1, wherein the content of the compound represented by formula (1) or a salt thereof is from 5 to 80 mol % based on the total lipid.

8. The method of claim 1, wherein the therapeutic agent is a nucleic acid.

9. The method of claim 1, wherein the ionizable lipid is a compound represented by formula (4): 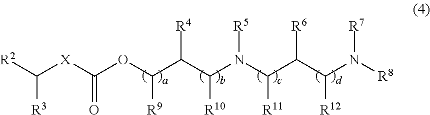
wherein X represents NR¹— or —O—,
R¹ represents a hydrogen atom, a hydrocarbon group having 6 to 24 carbon atoms, or a group represented by R²¹-L¹-R²²—, R²¹ represents a hydrocarbon group having 1 to 24 carbon atoms, and L¹ represents —O(CO)O—, —O(CO)—, —(CO)O—, —O—, or 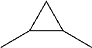
R²² is a divalent linking group and represents a hydrocarbon linking group having 1 to 18 carbon atoms,
R² and R³ each independently represent a hydrogen atom, a hydrocarbon group having 3 to 24 carbon atoms, or a group represented by R³¹-L²-R³²—, R³¹ represents a hydrocarbon group having 1 to 24 carbon atoms, and L² represents —O(CO)O—, —O(CO)—, —(CO)O—, —O—, or 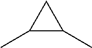
R³² is a divalent linking group and represents a hydrocarbon linking group having 1 to 18 carbon atoms,
each of R⁴, R⁵, R⁶, R⁷, R⁸, R⁹, R¹⁰, R¹¹, and R¹² independently represents a hydrogen atom or an optionally substituted alkyl group having 1-18 carbon atoms,
any one or more sets of R⁴ and R⁵, R¹⁰ and R⁵, R⁵ and R¹², R⁴ and R⁶, R⁵ and R⁶, R⁶ and R⁷, R⁶ and R¹⁰, R¹² and R⁷, and R⁷ and R⁸, may be linked together to form a 4- to 7-membered ring which may contain O atom,
a substituents on the optionally substituted alkyl group having 1-18 carbon atoms represents a hydroxyl groups, a carboxyl groups, an amino groups represented by NR⁴⁵R⁴⁶, a substituted or unsubstituted aryl group, a substituted or unsubstituted heteroaryl group, or a group represented by —O(CO)O—R⁴¹, —O(CO)—R⁴², —(CO)O—R⁴³, or —O—R⁴⁴, wherein R⁴¹, R⁴², R⁴³, R⁴⁴, R⁴⁵ and R⁴⁶ each independently represents a hydrocarbon group having 1 to 18 carbon atoms, a substituents on the substituted or unsubstituted aryl group and the substituted or unsubstituted heteroaryl group represents an alkyl groups having 1 to 18 carbon atoms, a hydroxyl groups, a carboxyl groups, an amino groups represented by —NR⁴⁵R⁴⁶, or a group represented by —O(CO)O—R⁴¹, —O(CO)—R⁴², —(CO)O—R⁴³, or —O—R⁴⁴, and R⁴¹, R⁴², R⁴³, R⁴⁴, R⁴⁵ and R⁴⁶ each independently represents a hydrocarbon group having 1 to 18 carbon atoms,
a, b, c, and d each independently represents an integer from 0 to 3, wherein a+b is 1 or more, and c+d is 1 or more.

10. The method of claim 1, wherein the ionizable lipid is a compound represented by formula (1): 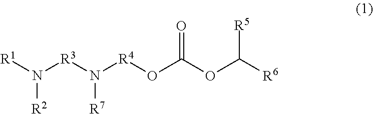
In the formula,
R¹ and R² each independently represent a hydrocarbon group having 1 to 18 carbon atoms, and R³ represents a hydrocarbon group having 2 to 8 carbon atoms, where the hydrocarbon groups represented by R¹, R², and R³ may be substituted with one or more substituents selected from —OH, COOH, —NR⁵¹R⁵², —OC(O)O—R⁵³, —C(O)O—R⁵⁴, —OC(O)—R⁵⁵, and —O—R⁵⁶,
R⁴ represents a hydrocarbon group having 1 to 8 carbon atoms,
R⁵ and R⁶ each independently represent a hydrocarbon group having 1 to 8 carbon atoms or —R⁸-L¹-R⁹, excluding a case that both R⁵ and R⁶ are hydrocarbon groups having 1 to 8 carbon atoms,
R⁷ represents —R¹⁰-L²-R¹¹-L³-R¹²,
R⁵¹ and R⁵² each independently represent a hydrocarbon group having 1 to 8 carbon atoms,
R⁵³, R⁵⁴, R⁵⁵, and R⁵⁶ each independently represent a hydrocarbon group having 1 to 24 carbon atoms,
the hydrocarbon groups represented by R⁵³, R⁵⁴, R⁵⁵, and R⁵⁶ may be substituted with an aryl group having 6 to 20 carbon atoms or —S—R⁵⁸,
the above-described aryl group having 6 to 20 carbon atoms may be substituted with —OH, COOH, —NR⁵¹R⁵², —OC(O)O—R⁵³, —C(O)O—R⁵⁴, —OC(O)—R⁵⁵, —O—R⁵⁶, or -(hydrocarbon group having 1 to 12 carbon atoms)-R⁵⁷,
R⁵⁸ represents a hydrocarbon group having 1 to 12 carbon atoms, and
R⁵⁷ represents —OH, COOH, —NR⁶¹R⁶², —OC(O)O—R⁶³, —C(O)O—R⁶⁴, —OC(O)—R⁶⁵, or —O—R⁶⁶,
R⁶¹ and R⁶² each independently represent a hydrocarbon group having 1 to 8 carbon atoms,
R⁶³, R⁶⁴, R⁶⁵, and R⁶⁶ each independently represent a hydrocarbon group having 1 to 24 carbon atoms,
the hydrocarbon groups represented by R⁶³, R⁶⁴, R⁶⁵, and R⁶⁶ may be substituted with an aryl group having 6 to 20 carbon atoms or —S—R⁶⁸,
the above-described aryl group having 6 to 20 carbon atoms may be substituted with —OH, COOH, —NR⁶¹R⁶², —OC(O)O—R⁶³, —C(O)O—R⁶⁴, —OC(O)—R⁶⁵, —O—R⁶⁶, or -(hydrocarbon group having 1 to 12 carbon atoms)-R⁶⁷,
R⁶⁸ represents a hydrocarbon group having 1 to 12 carbon atoms, and
L¹, L², and L³ each independently represent —OC(O)O—, —C(O)O—, —OC(O)—, or —O—,
R⁸ represents a hydrocarbon group having 1 to 12 carbon atoms,
R⁹ represents a hydrocarbon group having 1 to 24 carbon atoms,
R¹⁰ represents a hydrocarbon group having 1 to 8 carbon atoms,
R¹¹ represents a hydrocarbon group having 1 to 24 carbon atoms,
R¹² represents a hydrocarbon group having 1 to 24 carbon atoms,
the hydrocarbon groups represented by R⁹ and R¹² may be substituted with an aryl group, —OC(O)O—R⁵³, —C(O)O—R⁵⁴, —OC(O)—R⁵⁵, or —S—R⁵⁸, where definitions of R⁵³, R⁵⁴, R⁵⁵, and R⁵⁸ are as described above, and
the hydrocarbon group represented by R¹¹ may be substituted with —OC(O)O—R⁵³, —C(O)O—R⁵⁴, or —OC(O)—R⁵⁵, where the definitions of R⁵³, R⁵⁴, and R⁵⁵ are as described above.

11. The method of claim 1, wherein the ionizable lipid is a compound represented by the following formula (5): 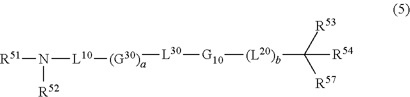
wherein R⁵¹ and R⁵² each independently represent a hydrocarbon group having 1 to 21 carbon atoms which may have a substituent A,
the substituent A represents a hydroxyl group, or a group represented by -G²⁰-CH(R⁵⁵)(R⁵⁶), —N(R⁵⁸)(R⁵⁹) or -G²⁰-R⁶⁰,
G²⁰ represents —O(CO)—, or —(CO)O—,
R⁵⁵ and R⁵⁶ each independently represent a hydrogen atom or a hydrocarbon group having 1 to 18 carbon atoms,
R⁵⁸ and R⁵⁹ each independently represent a hydrogen atom or a cyclic hydrocarbon group having 3 to 6 carbon atoms which may have a substituent B,
the substituent B is —N(R⁶¹)(R⁶²),
R⁶¹ and R⁶² each independently represent a hydrogen atom or a hydrocarbon group having 1 to 6 carbon atoms,
R⁶⁰ represents a hydrocarbon group having 1 to 18 carbon atoms,
L¹⁰ represents a hydrocarbon group having 1 to 18 carbon atoms,
G³⁰ indicates —S—(CO)—NR⁶⁴,
R⁶⁴ represents a group represented by -L³⁰-G²⁰-CH(R⁵⁵)(R⁵⁶),
a represents 0 or 1,
L³⁰ represents a single bond or a hydrocarbon group having 1 to 18 carbon atoms,
G¹⁰ represents —O(CO)—, —(CO)O—, —O(CO)O—, or —N(C(O)R⁶³)—,
R⁶³ represents a hydrocarbon group having 1 to 18 carbon atoms,
L²⁰ represents a hydrocarbon group having 1 to 6 carbon atoms,
b represents 0 or 1,
R⁵³, R⁵⁴ and R⁵⁷ each independently represent a hydrogen atom or a hydrocarbon group having 1 to 21 carbon atoms which may have a substituent C,
the substituent C represents a group represented by —(CO)O R⁶⁵ or —O(CO)—R⁶⁵,
R⁶⁵ represents a hydrocarbon group having 1 to 18 carbon atoms or a group represented by -L⁴⁰-CH(R⁶⁶)(R⁶⁷),
L⁴⁰ represents a hydrocarbon group having 1 to 6 carbon atoms,
R⁶⁶ and R⁶⁷ represent a hydrocarbon group having 1 to 10 carbon atoms or an alkoxy group.

12. The method of claim 1, wherein the lipid nanoparticle further comprises a lipid having a nonionic hydrophilic polymer and/or a phospholipid.

13. The method according to claim 1, wherein the lipid composition is administered to a subject by intravenous or intramuscular injection.

14. The method of claim 1, wherein the mesenchymal cell is myocyte, extracellular matrix producing cell, stellate cell, or fibroblast.

15. A lipid composition comprising a therapeutic agent and lipid nanoparticles, wherein the lipid nanoparticle comprises a compound represented by formula (1) or a salt thereof and an ionizable lipid having a biodegradable group, 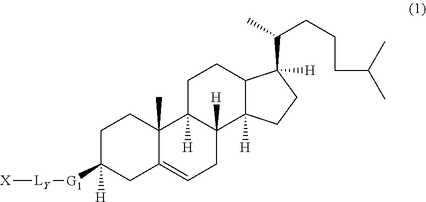
wherein G₁ represents —C(O)—, —OC(O)—, —O(CO)O— or —C(O)O—,
L_{Y} represents a single bond, an alkylene group having 1-14 carbon atoms, a substituted alkylene group having 1-14 carbon atoms, a heteroalkylene group having 1-14 carbon atoms, and a substituted heteroalkylene group having 1-14 carbon atoms,
X represents a basic functional group.

16. The lipid composition of claim 15, wherein the compound represented by formula (1) is a compound represented by formula (2) 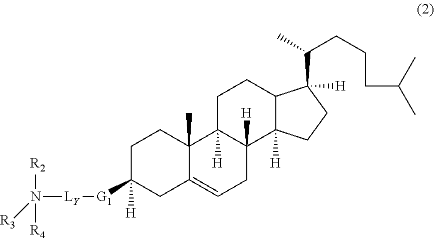
wherein G₁ represents —C(O)—, —OC(O)—, —O(CO)O— or —C(O)O—,
L_{Y} represents a single bond, an alkylene group having 1-14 carbon atoms, a substituted alkylene group having 1-14 carbon atoms, a heteroalkylene group having 1-14 carbon atoms,
and a substituted heteroalkylene group having 1-14 carbon atoms,
R², R³ and R⁴ each independently represents a hydrogen atom, a hydrocarbon group having 1 to 4 carbon atoms which may be substituted with a hydroxyl group, or —C(NH₂)=NH₂ and one of R², and
R³ and R⁴ may be absent.

17. The lipid composition according to claim 16, wherein the compound represented by formula (2) is a compound represented by formula (3) 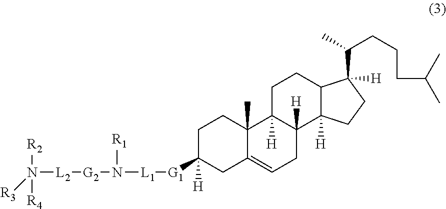
wherein G₁ represents —C(O)—, —OC(O)—, —O(CO)O— or —C(O)O—,
L₁ represents a single bond or an alkylene group having 1 to 6 carbon atoms,
R₁ represents a hydrogen atom, a hydrocarbon group containing 1 to 4 carbon atoms, or an aminoalkyl group having 1 to 4 carbon atoms,
G₂ represents a single bond, —C(O)—, —OC(O)—, —O(CO)O— or —C(O)O—,
L₂ represents an alkylene group having 1 to 6 carbons which may have an amino group,
R², R³ and R⁴ each independently represent a hydrogen atom, a hydrocarbon group having 1 to 4 carbon atoms which may be substituted with a hydroxyl group, or —C(NH₂)=NH₂, and one of R², R³ and R⁴ may be absent.

18. The lipid composition according to claim 15, wherein the ionizable lipid having a biodegradable group is a compound represented by formula (4) 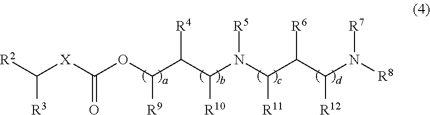
wherein X represents NR¹— or —O—,
R¹ represents a hydrogen atom, a hydrocarbon group having 6 to 24 carbon atoms, or a group represented by R²¹-L¹-R²²—, R²¹ represents a hydrocarbon group having 1 to 24 carbon atoms, and L¹ represents —O(CO)O—, —O(CO)—, —(CO)O—, —O—, or 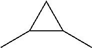
R²² is a divalent linking group and represents a hydrocarbon linking group having 1 to 18 carbon atoms,
R² and R³ each independently represent a hydrogen atom, a hydrocarbon group having 3 to 24 carbon atoms, or a group represented by R³¹-L²-R³²—, R³¹ represents a hydrocarbon group having 1 to 24 carbon atoms, and L² represents —O(CO)O—, —O(CO)—, —(CO)O—, —O—, or 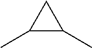
R³² is a divalent linking group and represents a hydrocarbon linking group having 1 to 18 carbon atoms,
each of R⁴, R⁵, R⁶, R⁷, R⁸, R⁹, R¹⁰, R¹¹, and R¹² independently represents a hydrogen atom or an optionally substituted alkyl group having 1-18 carbon atoms,
any one or more sets of R⁴ and R⁵, R¹⁰ and R⁵, R⁵ and R¹², R⁴ and R⁶, R⁵ and R⁶, R⁶ and R⁷, R⁶ and R¹⁰, R¹² and R⁷, and R⁷ and R⁸, may be linked together to form a 4- to 7-membered ring which may contain O atom,
a substituents on the optionally substituted alkyl group having 1-18 carbon atoms represents a hydroxyl groups, a carboxyl groups, an amino groups represented by NR⁴⁵R⁴⁶, a substituted or unsubstituted aryl group, a substituted or unsubstituted heteroaryl group, or a group represented by —O(CO)O—R⁴¹, —O(CO)—R⁴², —(CO)O—R⁴³, or —O—R⁴⁴, wherein R⁴¹, R⁴², R⁴³, R⁴⁴, R⁴⁵ and R⁴⁶ each independently represents a hydrocarbon group having 1 to 18 carbon atoms, a substituents on the substituted or unsubstituted aryl group and the substituted or unsubstituted heteroaryl group represents an alkyl groups having 1 to 18 carbon atoms, a hydroxyl groups, a carboxyl groups, an amino groups represented by —NR⁴⁵R⁴⁶, or a group represented by —O(CO)O—R⁴¹, —O(CO)—R⁴², —(CO)O—R⁴³, or —O—R⁴⁴, and R⁴¹, R⁴², R⁴³, R⁴⁴, R⁴⁵ and R⁴⁶ each independently represents a hydrocarbon group having 1 to 18 carbon atoms,
a, b, c, and d each independently represents an integer from 0 to 3, wherein a+b is 1 or more, and c+d is 1 or more; or
wherein the ionizable lipid is a compound re resented by formula (1): 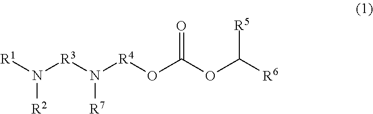
In the formula,
R¹ and R² each independently represent a hydrocarbon group having 1 to 18 carbon atoms, and R³ represents a hydrocarbon group having 2 to 8 carbon atoms, where the hydrocarbon groups represented by R¹, R², and R³ may be substituted with one or more substituents selected from —OH, COOH, —NR⁵¹R⁵², —OC(O)O—R⁵³, —C(O)O—R⁵⁴, —OC(O)—R⁵⁵, and —O—R⁵⁶,
R⁴ represents a hydrocarbon group having 1 to 8 carbon atoms,
R⁵ and R⁶ each independently represent a hydrocarbon group having 1 to 8 carbon atoms or —R⁸-L¹-R⁹, excluding a case that both R⁵ and R⁶ are hydrocarbon groups having 1 to 8 carbon atoms,
R⁷ represents —R¹⁰-L²-R¹¹-L³-R¹²,
R⁵¹ and R⁵² each independently represent a hydrocarbon group having 1 to 8 carbon atoms,
R⁵³, R⁵⁴, R⁵⁵, and R⁵⁶ each independently represent a hydrocarbon group having 1 to 24 carbon atoms,
the hydrocarbon groups represented by R⁵³, R⁵⁴, R⁵⁵, and R⁵⁶ may be substituted with an aryl group having 6 to 20 carbon atoms or —S—R⁵⁸,
the above-described aryl group having 6 to 20 carbon atoms may be substituted with —OH, COOH, —NR⁵¹R⁵², —OC(O)O—R⁵³, —C(O)O—R⁵⁴, —OC(O)—R⁵⁵, —O—R⁵⁶, or -(hydrocarbon group having 1 to 12 carbon atoms)-R⁵⁷,
R⁵⁸ represents a hydrocarbon group having 1 to 12 carbon atoms, and
R⁵⁷ represents —OH, COOH, —NR⁶¹R⁶², —OC(O)O—R⁶³, —C(O)O—R⁶⁴, —OC(O)—R⁶⁵, or —O—R⁶⁶,
R⁶¹ and R⁶² each independently represent a hydrocarbon group having 1 to 8 carbon atoms,
R⁶³, R⁶⁴, R⁶⁵, and R⁶⁶ each independently represent a hydrocarbon group having 1 to 24 carbon atoms,
the hydrocarbon groups represented by R⁶³, R⁶⁴, R⁶⁵, and R⁶⁶ may be substituted with an aryl group having 6 to 20 carbon atoms or —S—R⁶⁸,
the above-described aryl group having 6 to 20 carbon atoms may be substituted with —OH, COOH, —NR⁶¹R⁶², —OC(O)O—R⁶³, —C(O)O—R⁶⁴, —OC(O)—R⁶⁵, —O—R⁶⁶, or -(hydrocarbon group having 1 to 12 carbon atoms)-R⁶⁷,
R⁶⁸ represents a hydrocarbon group having 1 to 12 carbon atoms, and
L¹, L², and L³ each independently represent —OC(O)O—, —C(O)O—, —OC(O)—, or —O—,
R⁸ represents a hydrocarbon group having 1 to 12 carbon atoms,
R⁹ represents a hydrocarbon group having 1 to 24 carbon atoms,
R¹⁰ represents a hydrocarbon group having 1 to 8 carbon atoms,
R¹¹ represents a hydrocarbon group having 1 to 24 carbon atoms,
R¹² represents a hydrocarbon group having 1 to 24 carbon atoms,
the hydrocarbon groups represented by R⁹ and R¹² may be substituted with an aryl group, —OC(O)O—R⁵³, —C(O)O—R⁵⁴, —OC(O)—R⁵⁵, or —S—R⁵⁸, where definitions of R⁵³, R⁵⁴, R⁵⁵, and R⁵⁸ are as described above, and
the hydrocarbon group represented by R¹¹ may be substituted with —OC(O)O—R⁵³, —C(O)O—R⁵⁴, or —OC(O)—R⁵⁵, where the definitions of R⁵³, R⁵⁴, and R⁵⁵ are as described above; or
wherein the ionizable lipid having a biodegradable group is a compound represented by Formula (5): 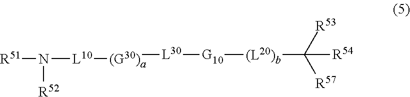
wherein R⁵¹ and R⁵² each independently represent a hydrocarbon group having 1 to 21 carbon atoms which may have a substituent A,
the substituent A represents a hydroxyl group, or a group represented by -G²⁰-CH(R⁵⁵)(R⁵⁶), —N(R⁵⁸)(R⁵⁹) or -G²⁰-R⁶⁰,
G²⁰ represents —O(CO)—, or —(CO)O—,
R⁵⁵ and R⁵⁶ each independently represent a hydrogen atom or a hydrocarbon group having 1 to 18 carbon atoms,
R⁵¹ and R⁵⁹ each independently represent a hydrogen atom or a cyclic hydrocarbon group having 3 to 6 carbon atoms which may have a substituent B,
the substituent B is —N(R⁶¹)(R⁶²),
R⁶¹ and R⁶² each independently represent a hydrogen atom or a hydrocarbon group having 1 to 6 carbon atoms,
R⁶⁰ represents a hydrocarbon group having 1 to 18 carbon atoms,
L¹⁰ represents a hydrocarbon group having 1 to 18 carbon atoms,
G³⁰ indicates —S—(CO)—NR⁶⁴,
R⁶⁴ represents a group represented by -L³⁰-G²⁰-CH(R⁵⁵)(R⁵⁶) a represents 0 or 1,
L³⁰ represents a single bond or a hydrocarbon group having 1 to 18 carbon atoms,
G¹⁰ represents —O(CO)—, —(CO)O—, —O(CO)O—, or —N(C(O)R⁶³)—,
R⁶³ represents a hydrocarbon group having 1 to 18 carbon atoms,
L²⁰ represents a hydrocarbon group having 1 to 6 carbon atoms,
b represents 0 or 1,
R⁵³, R⁵⁴ and R⁵⁷ each independently represent a hydrogen atom or a hydrocarbon group having 1 to 21 carbon atoms which may have a substituent C,
the substituent C represents a group represented by —(CO)O R⁶⁵ or —O(CO)—R⁶⁵,
R⁶⁵ represents a hydrocarbon group having 1 to 18 carbon atoms or a group represented by -L⁴⁰-CH(R⁶⁶)(R⁶⁷),
L⁴⁰ represents a hydrocarbon group having 1 to 6 carbon atoms,
R⁶⁶ and R⁶⁷ represent a hydrocarbon group having 1 to 10 carbon atoms or an alkoxy group.

19. The lipid composition of claim 15, wherein the content of the compound represented by Formula (1) or a salt thereof is from 5 to 80 mol % based on the total lipid.

20. The lipid composition of claim 15, wherein the therapeutic agent is a nucleic acid.
